SEQUENCE LISTING (60 sequences)

SEQ ID NO: 1 - DNA, 24 bp, Human
atgtggctccggagccatcgtcag

SEQ ID NO: 2 - DNA, 24 bp, Human
aggatctctcaagtccgcgtactc

SEQ ID NO: 3 - DNA, 24 bp, Human
ctagcgaagattcaagccatgtgg

SEQ ID NO: 4 - DNA, 21 bp, Human
gacgtactcccccgggatgtg

SEQ ID NO: 5 - DNA, 24 bp, Human
atggatcccctgggtgcagccaag

SEQ ID NO: 6 - DNA, 24 bp, Human
gtgcccgaaattgggctcctgcac

SEQ ID NO: 7 - DNA, 24 bp, Human
gtgcccgaaattgggctcctgcac

SEQ ID NO: 8 - DNA, 24 bp, Human
gaaggaggtgatgtggacagcgta

SEQ ID NO: 9 - DNA, 24 bp, Human
cttatggcagtggaacctgtcacc

SEQ ID NO: 10 - DNA, 24 bp, Human
ccagccgtagggcgtgaagatgtc

SEQ ID NO: 11 - DNA, 24 bp, Human
cccactgtgtaccctaatgggtcc

SEQ ID NO: 12 - DNA, 24 bp, Human
ctctcaggtgaaccaagccgctat

SEQ ID NO: 13 - DNA, 22 bp, Human
tcggacatctttgtgccctatg

SEQ ID NO: 14 - DNA, 23 bp, Human
cgccagaatttctccgtaatgta

SEQ ID NO: 15 - DNA, 32 bp, Human
atctggcaccacaccttctacaatgagctgcg

SEQ ID NO: 16 - DNA, 32 bp, Human
cgtcatactcctgcttgctgatccacatctgc

SEQ ID NO: 17 - DNA, 32 bp, Human; identical to SEQ ID NO: 15

SEQ ID NO: 18 - DNA, 32 bp, Human; identical to SEQ ID NO: 16

SEQ ID NO: 19 - DNA, 27 bp, Human
cgauucgcuagaccggcuucauugcag

SEQ ID NO: 20 - DNA, 27 bp, Human
gcaaugaagccggucuagcgaaucgau

SEQ ID NO: 21 - DNA, 25 bp, Human
ccuuauaccuaacgacagacccuuu

SEQ ID NO: 22 - DNA, 25 bp, Human
aaagggucugucguuagguauaagg

SEQ ID NO: 23 - DNA, 25 bp, Human
ccuccauauccaucaagacagcuuu

SEQ ID NO: 24 - DNA, 25 bp, Human
aaagcugucuugauggauauggagg

SEQ ID NO: 25 - DNA, 25 bp, Human
cggacuugagagauccuuuccugaa

SEQ ID NO: 26 - DNA, 25 bp, Human
uucaggaaaggaucucucaaguccg

SEQ ID NO: 27 - DNA, 25 bp, Human
cacucugucccgguuuccuucagca

SEQ ID NO: 28 - DNA, 25 bp, Human
ugcugaaggaaaccgggacagagug

SEQ ID NO: 29 - DNA, 25 bp, Human
caucucucuucugugaagaugcguu

SEQ ID NO: 30 - DNA, 25 bp, Human
aacgcaucuucacagaagagagaug

SEQ ID NO: 31 - DNA, 25 bp, Human
ccgcacugcuauuucagcugcuggu

SEQ ID NO: 32 - DNA, 25 bp, Human
accagcagcugaaauagcagugcgg

SEQ ID NO: 33 - DNA, 25 bp, Human
cagacacggucaucgugcaccacug

SEQ ID NO: 34 - DNA, 25 bp, Human
caguggugcacgaugaccgugucug

SEQ ID NO: 35 - DNA, 25 bp, Human
cgaagccuggcaaguaaccucuuca

SEQ ID NO: 36 - DNA, 25 bp, Human
ugaagagguuacuugccaggcuucg

SEQ ID NO: 37 - DNA, 25 bp, Human
gcuacaaguucuaccuggcuuucga

SEQ ID NO: 38 - DNA, 25 bp, Human
ucgaaagccagguagaacuuguagc

SEQ ID NO: 39 - DNA, 25 bp, Human
uaggccagggcuuauggcaguggaa

SEQ ID NO: 40 - DNA, 25 bp, Human
uuccacugccauaagcccuggccua

SEQ ID NO: 41 - DNA, 25 bp, Human
caucgugcaccacugggauaucaug

SEQ ID NO: 42 - DNA, 25 bp, Human
caugauaucccaguggugcacgaug

SEQ ID NO: 43 - DNA, 25 bp, Human
gcugucugaccacgcugcuguuuca

SEQ ID NO: 44 - DNA, 25 bp, Human
ugaaacagcagcguggucagacagc

SEQ ID NO: 45 - DNA, 25 bp, Human
acacgcggcauagcggcuugguuca

SEQ ID NO: 46 - DNA, 25 bp, Human
ugaaccaagccgcuaugccgcgugu

SEQ ID NO: 47 - DNA, 25 bp, Human
cgccucaucugcggguggaugucuu

SEQ ID NO: 48 - DNA, 25 bp, Human
aagacauccacccgcagaugaggcg

SEQ ID NO: 49 - DNA, 25 bp, Human
gcgggaacguuucugugccaucugu

SEQ ID NO: 50 - DNA, 25 bp, Human
acagauggcacagaaacguucccgc

SEQ ID NO: 51 - DNA, 25 bp, Human
caucccaggucugucgaguugcuua

SEQ ID NO: 52 - DNA, 25 bp, Human
uaagcaacucgacagaccugggaug

SEQ ID NO: 53 - DNA, 25 bp, Human
gagauaucauugguguggcuggaaa

SEQ ID NO: 54 - DNA, 25 bp, Human
uuuccagccacaccaaugauaucuc

SEQ ID NO: 55 - DNA, 20 bp, Human
cttaccgccgtgattcagat

SEQ ID NO: 56 - DNA, 20 bp, Human
aatgcttgcccgtaggtatg

SEQ ID NO: 57 - DNA, 22 bp, Human
tcggacatctttgtgccctatg

SEQ ID NO: 58 - DNA, 20 bp, Human
tttcagtggcctccagaact

SEQ ID NO: 59 - DNA, 20 bp, Human
gaaggaaggaaacccctttg

SEQ ID NO: 60 - DNA, 20 bp, Human
tctcccgtcttcaccatttc

(19) United States
(12) Patent Application Publication
(10) Pub. No.: US 2015/0017098 A1
(43) Pub. Date: Jan. 15, 2015

(54) CARRIER THAT TARGETS FUCOSYLATED MOLECULE-PRODUCING CELLS

(71) Applicants: Junji Kato, Sapporo-shi (JP); Rishu Takimoto, Sapporo-shi (JP)

(72) Inventors: Junji Kato, Sapporo-shi (JP); Rishu Takimoto, Sapporo-shi (JP)

(21) Appl. No.: 13/278,159

(22) Filed: Oct. 20, 2011

Related U.S. Application Data
(60) Provisional application No. 61/410,433, filed on Nov. 5, 2010.

(51) Int. Cl. A61K 47/26; A61K 31/704; A61K 33/24; A61K 49/00; A61K 9/127; C12N 15/113

(52) U.S. Cl. CPC A61K 47/26; A61K 9/127; C12N 15/113; A61K 33/24; A61K 49/0052; A61K 31/704; C12N 2320/32
USPC 424/1.73; 536/1.11; 424/450; 514/777; 424/489; 424/9.35; 424/9.6; 514/44 A; 424/649; 514/34; 435/188

(57) ABSTRACT

The present invention relates to a carrier that is targeted at fucosylated molecule-producing cells, which comprises an effective amount of fucose for targeting said cells, to a composition comprising the carrier, and to a method for treating and diagnosing a disease related to fucosylated molecule-producing cells utilizing said carrier, etc. The carrier of the present invention enables to deliver a substance specifically to fucosylated molecule-producing cells.

### TECHNICAL FIELD

The present invention relates to a carrier that targets at fucosylated molecule-producing cells, a treatment agent and a treatment method of a disease related to the fucosylated molecule-producing cells utilizing said carrier, and a detection agent and a detection method of the fucosylated molecule-producing cells utilizing said carrier, etc.

### BACKGROUND ART

In eucaryote, it has been known that fucosylated sugar chains are involved in various physiological and pathological processes including angiogenesis, reproduction, cell adhesion, inflammation and tumor metastasis (see Non-patent Literature 1). In addition, a number of glycoprotein tumor markers including CA19-9 and SLX are known to be generated by fucosylation of sugar chains (see Non-patent Literature 2). Thus, because fucosylated sugar chains have a significant implication in organisms, if a substance such as a drug can be specifically delivered to cells producing fucosylated sugar chains, then the above-mentioned various phenomena can be controlled. However, to date there has been no report indicating a success of such an attempt.

Furthermore, since fucosylation is catalyzed by a kind of glycosyltransferase, i.e., fucosyltransferase (FUT), one may imagine targeting fucosylated sugar chain-producing cells by using a fucosyltransferase as a target molecule; however, this enzyme is a membrane-bound protein localized at regions from the endoplasmic reticulum to the Golgi apparatus, and is not present on the cell surface; accordingly, fucosyltransferases cannot be used as a direct target molecule. Consequently, a technology to deliver a substance such as a drug specifically to fucosylated sugar chain-producing cells has not been developed to date.

### CITATION LIST

### Patent Literature

### Patent Literature 1: JP A 2009-46441

### Patent Literature 2: JP A 2004-522722

### Non-Patent Literature

### Non-patent Literature 1: Ma et al., Glycobiology. 2006; 16(12): 158R-184R.

### Non-patent Literature 2: Ma et al., Glycobiology. 1998; 8(6): 605-13.

### Non-patent Literature 3: Kawakami et al., Biochem Biophys Acta. 2000; 1524(2-3): 258-65.

### SUMMARY OF INVENTION

### Problem to be Solved by Invention

An object of the present invention is to provide a carrier that can deliver a substance such as a drug specifically to fucosylated molecule-producing cells, a treatment agent of a disease related to the fucosylated molecule-producing cells and a treatment method of a disease related to the fucosylated molecule-producing cells, utilizing said carrier, etc.

### Means of Solving Problems

The present inventors have devoted themselves to the research to solve the above problem, and found that there exists in fucosylated molecule-producing cells a mechanism to specifically bind fucose. After performing further research based on this finding, the inventors have found that a carrier comprising fucose specifically facilitates the delivery of a substance to fucosylated molecule-producing cells, and accomplished this invention.

The presence of a mechanism to specifically bind fucose in fucosylated molecule-producing cells has not at all been known to date. Moreover, although a carrier comprising fucose has been known (see Patent Literatures 1 and 2, Non-patent Literature 3), the fact that this carrier specifically facilitates the delivery of a substance to fucosylated molecule-producing cells has not been known to date.

Namely, the present invention relates to the following.

(i) A carrier targeting fucosylated molecule-producing cells, which comprises an effective amount of fucose for targeting said cells.
(ii) The carrier according to the above (i), wherein the fucose is L-fucose.
(iii) The carrier according to the above (i) or (ii), wherein the fucosylated molecule comprises a type I sugar chain.
(iv) The carrier according to the above (i) or (ii), wherein the fucosylated molecule comprises O-linked fucose.
(v) The carrier according to any one of the above (i)-(iv), wherein the fucosylated molecule-producing cell expresses a fucosyltransferase.
(vi) The carrier according to the above (v), wherein the fucosyltransferase is selected from the group consisting of FUT1, FUT2, FUT3, FUT4, FUT5, FUT6, FUT7, FUT8, FUT9, FUT10, FUT11, POFUT1, and POFUT2.
(vii) The carrier according to any one of the above (i)-(vi), wherein the carrier has a form selected from polymer micelle, liposome, emulsion, microsphere, and nanosphere.
(viii) The carrier according to any one of the above (i)-(vii), wherein the carrier has a form of liposome, and the molar ratio of the fucose to the lipid contained in the liposome is 8:1-1:8.
(ix) A composition comprising the carrier according to any one of the above (i)-(viii) and a drug that controls the activity or growth of fucosylated molecule-producing cells.
(x) The composition according to the above (ix), wherein the drug that controls the activity or growth of fucosylated molecule-producing cells is selected from the group consisting of anti-inflammatory agents and antitumor agents.
(xi) The composition according to the above (ix) or (x), wherein the composition is prepared by mixing the drug and the carrier at a site of clinical practice or its vicinity.
(xii) A composition comprising the carrier according to any one of the above (i)-(viii) and a label.
(xiii) The composition according to the above (xii), wherein the label is selected from the group consisting of a gas or a substance that generates a gas under physiological conditions, a radioisotope, a magnetic substance, a nuclear magnetic resonance atom, a substance that affects the relaxation time of a nuclear magnetic resonance atom, a substance that binds to a labeling substance, a fluorescent substance, a fluorophore, a chemiluminescent substance, an enzyme, biotin or its derivative, avidin or its derivative, or a substance comprising one or more thereof.
(xiv) A preparation kit for the composition according to any one of the above (ix)-(xi), containing one or more containers that contain a drug that controls the activity or growth of fucosylated molecule-producing cells, a fucose donor, and as necessary, a carrier-constitutive substance other than fucose, singly or in a combination thereof.
(xv) A method for treating a disease related to fucosylated molecule-producing cells, comprising administering to a subject in need thereof the composition according to any one of the above (ix)-(xi) in an amount effective for treating said disease.
(xvi) The method according to the above (xv), wherein the disease is selected from the group consisting of neoplastic diseases and inflammatory diseases.
(xvii) The method according to the above (xvi), wherein the neoplastic disease is selected from the group consisting of solid tumors and leukemia.
(xviii) A method for detecting fucosylated molecule-producing cells in a subject, comprising administering to the subject in need thereof the composition according to the above (xii) or (xiii) in an amount effective for the detection.
(xix) The method according to the above (xviii), wherein the cell is detected by imaging.
(xx) The method according to the above (xviii) or (xix), wherein the cell is selected from the group consisting of neoplastic cells and inflammatory cells.
(xxi) A method for diagnosing a disease related to fucosylated molecule-producing cells, comprising administering to a subject in need thereof the composition according to the above (xii) or (xiii) in an amount effective for detection.
(xxii) A method for delivering a substance to fucosylated molecule-producing cells, utilizing the carrier according to any one of the above (i) to (viii).

Furthermore, the present invention also relates to the following.

(1) A substance delivery carrier for fucosylated sugar chain-producing cells, which comprises fucose.
(2) The carrier according to the above (1), wherein the fucose is L-fucose.
(3) The carrier according to the above (1) or (2), wherein the fucosylated sugar chain is a type I sugar chain.
(4) The carrier according to any one of the above (1) to (3), wherein the fucosylated sugar chain-producing cell expresses a fucosyltransferase.
(5) The carrier according to the above (4), wherein the fucosyltransferase is selected from the group consisting of FUT1, FUT2, FUT3 and FUT4.
(6) The carrier according to any one of the above (1) to (5), wherein the carrier has a form selected from polymer micelle, liposome, emulsion, microsphere, and nanosphere.
(7) The carrier according to the above (6), wherein the carrier has a form of liposome, and the molar ratio of the fucose to the lipid contained in the liposome is 8:1-1:8.
(8) A composition for treating a disease related to fucosylated sugar chain-producing cells, comprising the carrier according to any one of the above (1) to (7) and a drug for the treatment of a disease related to fucosylated sugar chain-producing cells.
(9) The composition according to the above (8), wherein the disease is selected from the group consisting of neoplastic diseases and inflammatory diseases.
(10) The composition according to the above (9), wherein the drug for the treatment of a disease related to fucosylated sugar chain-producing cells is selected from the group consisting of anti-inflammatory agents and antitumor agents.
(11) The composition according to any one of the above (8) to (10), wherein the composition is prepared by mixing the drug and the carrier at a site of clinical practice or its vicinity.
(12) A preparation kit for the composition according to any one of the above (8) to (11), containing one or more containers that contain a drug for the treatment of a disease related to fucosylated sugar chain-producing cells, fucose, and as necessary, a carrier-constitutive substance other than fucose, singly or in a combination thereof.
(13) A method for delivering a substance to fucosylated sugar chain-producing cells in vitro, utilizing the carrier according to any one of the above (1) to (7).

### Advantageous Effects of Invention

The carrier of the present invention specifically targets at the fucose-binding mechanism possessed by fucosylated molecule-producing cells; the carrier enables to achieve desired effects including, for example, suppression of activity and growth of fucosylated molecule-producing cells, and to cure, to inhibit the progression, or to prevent the onset or recurrence of a disease related to fucosylated molecule-producing cells, with maximum effects and minimum side effects, by means of efficiently transporting desired substances and matters, such as a label or a drug for treating diseases related to fucosylated molecule-producing cells, to said cells.

In addition, because the carrier of the present invention enables specific delivery of a substance to fucosylated molecule-producing cells, it can be used for the fucosylated molecule-producing cell-specific labeling and gene introduction.

### BRIEF DESCRIPTION OF DRAWINGS

FIG. 1 is a graph showing concentrations of CA 19-9, SPAN-1 and DU-PAN-2 in a supernatant of various pancreatic cancer cell line cultures.

FIG. 2 shows expression states of various fucosyltransferases in various pancreatic cancer cell lines.

FIG. 3 is a graph showing the relationship between the amount of fucose binding and the concentration of free fucose in fucosylated sugar chain high-producing cell line ASPC-1 (upper graph) and in fucosylated sugar chain low-producing cell line PANC-1 (lower graph)

FIG. 4 is a graph showing calculation results of the binding constants Kd and Bmax for fucose of the fucosylated sugar chain high-producing cell line ASPC-1 (upper graph) and of the fucosylated sugar chain low-producing cell line PANC-1 (lower graph), based on the results shown in FIG. 3.

FIG. 5 is a graph showing binding of ¹⁴C fucose in the fucosylated sugar chain high-producing cell line ASPC-1, and its inhibition by the competition with an excessive amount of non-labeled fucose.

FIG. 6 shows photographs showing introduction of siRNA by fucosylated liposomes with various molar ratios (fucose/liposome).

FIG. 7 shows photographs showing introduction of siRNA by fucosylated liposomes with various molar ratios (fucose/liposome).

FIG. 8 shows photographs showing effects of addition of fucose on the introduction of siRNA by fucosylated liposomes. The photographs indicate the results for non-treatment group, non-fucosylated liposome treatment group, fucosylated liposome treatment group, fucosylated liposome treatment group with addition of an excessive amount of fucose, from the left, respectively.

FIG. 9 shows photographs indicating the comparison of siRNA-introduction efficiency in various pancreatic cancer cell lines. The photographs indicate the results for non-treatment group (upper photos), non-fucosylated liposome treatment group (middle photos), and fucosylated liposome treatment group (lower photos), respectively.

FIG. 10 shows photographs indicating the comparison of siRNA-introduction efficiency in various pancreatic cancer cell lines. The photographs indicate the results for non-treatment group (upper photos), non-fucosylated liposome treatment group (middle photos), and fucosylated liposome treatment group (lower photos), respectively.

FIG. 11 is a graph showing fucosyltransferase-dependent production of CA19-9 in a pancreatic cancer cell line (AsPC-1). FIG. 11(a) shows inhibition of expression of FUT genes by siRNA. FIG. 11(b) shows secretion of CA19-9 in a cell transfected with FUT-siRNA.

FIG. 12 shows a scheme of CDDP encapsulation (inclusion) using CDDP3. FIGS. 12(a) and (b) indicate chemical structures of CDDP and CDDP3, respectively. FIG. 12(c) shows CDDP3 in TAPS buffer (pH 8.4) that does not comprise NaCl. FIG. 12(d) indicates CDDP3 in a reversible equilibrium state with coordination of H₂O molecules due to high solubility in water. In the step of FIG. 12(e), CDDP3 is taken up by a liposome, and shows various molecular forms in the liposome. In (f), by changing to TAPS buffer (pH 8.4) that comprises 150 mM of NaCl, chlorine ions flow into the liposome and preferentially form a coordination bond to produce CDDP.

FIG. 13 is a diagram showing preparation of fucosylated liposomes. FIG. 13(a) is a scheme for the preparation of fucosylated liposomes. FIG. 13(b) is an electron micrograph of fucosylated liposomes (the bar represents 100 nm).

FIG. 14 shows graphs representing physiological properties of Cy5.5-included fucosylated liposomes. The average particle size (a) and zeta potential (b) of the liposomes prepared in water were measured by a dynamic light scattering photometer.

FIG. 15 shows photographs showing introduction of Cy5.5 encapsulated in fucosylated liposomes (magnification: 200×).

FIG. 16 shows graphs representing results of flow cytometry of cells treated with Cy5.5-included fucosylated liposomes. AsPC-1 cells (CA19-9 producing cells) (a) and PANC-1 cells (CA19-9 non-producing cells) (b) were treated with Cy5.5-included fucosylated liposomes for 2 hr under the presence (in the figure, +Fuc×100) or absence of excess fucose, and analyzed by flow cytometry.

FIG. 17 shows graphs representing effects of CDDP-encapsulated fucosylated liposomes on various types of pancreatic cancer cell lines. The cells were treated with CDDP-encapsulated fucosylated liposomes for 2 hr, washed, and incubated for 72 hr. Viable cells were measured by WST assay. In FIGS. 17(a) and (b), the vertical axis represents “% of control,” and the horizontal axis represents μM.

FIG. 18 is a graph showing CA19-9 concentrations in a supernatant of various types of colorectal cancer cell line cultures.

FIG. 19 shows photographs showing introduction of Cy5.5 encapsulated in fucosylated liposomes (magnification: 200×).

FIG. 20 shows graphs representing results of flow cytometry of cells treated with Cy5.5-included fucosylated liposomes.

FIG. 21 shows graphs representing effects of fucosylated liposomes that encapsulate CDDP on various types of colorectal cancer cell lines. The cells were treated with CDDP-encapsulated fucosylated liposomes for 2 hr, washed, and incubated for 72 hr. Viable cells were measured by WST assay. The vertical axis of the graphs represents “% of control.”

FIG. 22 shows graphs representing effects of fucosylated liposomes that encapsulate CDDP on various types of colorectal cancer cell lines. The cells were treated with CDDP-encapsulated fucosylated liposomes for 2 hr, washed, and incubated for 72 hr. Viable cells were measured by WST assay. The vertical axis of the graphs represents “% of control.”

FIG. 23 is a graph showing CA19-9 concentrations in a supernatant of various types of biliary tract cancer cell line cultures.

FIG. 24 shows graphs representing results of flow cytometry of cells treated with Cy5.5-included fucosylated liposomes.

FIG. 25 shows results of flow cytometry of COLO205 cells, i.e., CA19-9 high-producing stomach cancer cell line, treated with Cy5.5-included fucosylated liposomes, as well as fluorescence microscopic images.

FIG. 26 shows results of flow cytometry of MKN45 cells, i.e., CA19-9 non-producing stomach cancer cell line, treated with Cy5.5-included fucosylated liposomes, as well as fluorescence microscopic images.

FIG. 27 shows graphs representing effects of CDDP-encapsulated fucosylated liposomes on CA19-9 high-producing stomach cancer cell line COLO205 cells. The cells were treated with CDDP-encapsulated fucosylated liposomes for 1 hr, washed, and incubated for 72 hr. Viable cells were measured by WST assay.

FIG. 28 shows graphs representing effects of CDDP-encapsulated fucosylated liposomes on CA19-9 non-producing stomach cancer cell line MKN45 cells. The cells were treated with CDDP-encapsulated fucosylated liposomes for 1 hr, washed, and incubated for 72 hr. Viable cells were measured by WST-1 assay.

FIG. 29 is a diagram showing expression of CD33 and Notch-1 in various types of leukemic cell lines.

FIG. 30 is a diagram showing expression of fucosyltransferase in various types of leukemic cell lines.

FIG. 31 shows graphs representing results of flow cytometry of cells of Notch-1 expressing leukemic cell line (HL-60) and Notch-1 non-expressing leukemic cell line (MOLT-4), both treated with fluorescent label-included fucosylated liposomes.

FIG. 32 shows fluorescence microscopic images of cells of Notch-1 expressing leukemic cell line (HL-60) and Notch-1 non-expressing leukemic cell line (MOLT-4), both treated with FAM-included fucosylated liposomes.

FIG. 33 shows graphs representing effects of doxorubicin-encapsulated fucosylated liposomes on cells of Notch-1 expressing leukemic cell line (HL-60) and Notch-1 non-expressing leukemic cell line (MOLT-4). The cells were treated with doxorubicin-encapsulated fucosylated liposomes for 2 hr, washed, and incubated for 72 hr. Viable cells were measured by WST-1 assay.

FIG. 34 is a diagram showing states of expression of CD33 and Notch-1 in samples from leukemia patients.

FIG. 35 is a graph showing effects of doxorubicin-encapsulated fucosylated liposomes on Notch-1 expressing leukemic cells and Notch-1 non-expressing leukemic cells derived from samples of leukemia patients. The cells were treated with doxorubicin-encapsulated fucosylated liposomes for 2 hr, washed, and incubated for 72 hr. Viable cells were measured by WST-1 assay.

### DESCRIPTION OF EMBODIMENTS

One aspect of the present invention relates to a substance delivery carrier for fucosylated molecule-producing cells, which comprises fucose. One embodiment of the present invention relates to a carrier that is targeted at fucosylated molecule-producing cells by fucose. The carrier may comprise an effective amount of fucose for the targeting of fucosylated molecule-producing cells. Accordingly, one embodiment of the present invention relates to a carrier comprising an effective amount of fucose for the targeting of fucosylated molecule-producing cells, which is targeted at said cells (or which targets at said cells). In addition, the carrier may be used to deliver a substance to fucosylated molecule-producing cells. Accordingly, one embodiment of the present invention relates to a carrier for delivering a substance to fucosylated molecule-producing cells, which comprises fucose.

In the present invention, fucosylated molecule-producing cells (hereinafter, also referred to as target cells) are not particularly limited as long as they are the cells that produce fucosylated molecules; they may be those comprising fucosylated molecules on their cell surface, or inside the cells, or they may be those releasing fucosylated molecules outside the cell. Therefore, examples of the fucosylated molecule-producing cells of the present invention include, but are not limited to, cells in tumors, for example, pancreatic tumor, biliary system tumor, liver tumor, digestive tract tumor, brain tumor, lung tumor, bone and soft tissue tumor, hematopoietic organ tumor, more specifically, pancreatic cancer, biliary system cancer, liver cancer, stomach cancer, esophageal cancer, colorectal cancer, and further, breast cancer, lung cancer, endometrial cancer, prostate cancer, leukemia, lymphoma, etc., as well as cells at the site of inflammation in inflammatory diseases such as pancreatitis, cirrhosis, hepatitis, etc., and cells in the immune system such as lymphocytes. Examples of the cells at the site of inflammation include, but are not limited to, cells that are originally present at the site of inflammation and are affected by inflammation. Namely, cells at the site of inflammation refer to, in the case of pancreatitis, constitutive cells of the pancreas affected by the inflammation (ductal cells, exocrine cells, endocrine cells, etc.), and in the case of hepatitis, constitutive cells of the liver affected by the inflammation (hepatocytes, bile duct cells, Kupffer cells, stellate cells, etc.). Examples of the influence of inflammation include exposure to inflammatorycytokines and contact with inflammatory cells. Examples of leukemia include, but are not limited to, acute myeloid leukemia (AML), acute lymphatic leukemia (ALL), chronic myeloid leukemia (CML), chronic lymphatic leukemia (CLL), etc.

In one embodiment of the present invention, the fucosylated molecule-producing cell is preferably a cell other than normal cells. Examples of such a cell include the above tumor cells and the cells at the site of inflammation.

In the present invention, fucosylated molecules refer to any molecules to which fucose is added, and examples include, but are not limited to, fucosylated sugar chains, fucosylated glycoproteins, fucosylated glycolipids, etc. The number of fucose added is not particularly limited, and it may be 1, or 2 or more. Accordingly, fucosylated glycoproteins and fucosylated glycolipids may, as a sugar moiety, comprise fucose alone, or comprise a sugar chain that comprises fucose as a constituent sugar, i.e., a fucosylated sugar chain.

In the present invention, the fucosylated sugar chain may be produced as a single sugar chain, or in a form wherein a sugar chain is bound to other substances. Therefore, the fucosylated sugar chain may be produced in a form of glycoprotein wherein a sugar chain is bound to a protein, or produced in a form of glycolipid wherein a sugar chain is bound to a lipid. Moreover, the fucosylated sugar chain in the present invention may be a sugar chain with any structure as long as it contains fucose; however, those wherein fucose is contained at a non-reducing terminal are preferred. Fucose contained may be L-fucose or D-fucose, and L-fucose is preferable. In addition, the fucosylated sugar chain may comprise a type I carbohydrate antigen (such as CA19-9, SPAN-1, DU-PAN-2, CA50, KMO-1, etc.), or comprise a type II carbohydrate antigen (SLX, CSLEX, etc.), or comprise a mother nucleus carbohydrate antigen (such as CA72-4, CA546, STN, etc.). In one embodiment of the present invention, a sugar chain comprising a type I carbohydrate antigen is preferred. In addition, fucosylation may be carried out with various binding modes, for example, α1,2 linkage, α1,3 linkage, α1,4 linkage, and α1,6 linkage, etc. Of these, α1,4 linkage is preferred in the present invention. The sugar chain that is particularly preferred in the present invention includes a carbohydrate antigen selected from the group consisting of CA19-9, SPAN-1, and DU-PAN-2.

The fucosylated glycoprotein in the present invention includes any glycoprotein that comprises fucose in its sugar moiety, and examples include, but are not limited to, Notch receptors (Notch-1, Notch-2, Notch-3, Notch-4, etc.), Notch ligands (Delta-1, Delta-3, Delta-4, Jagged-1, Jagged-2, etc.), haptoglobin, AFP (α fetoprotein)-L3. Fucose may be added to the sugar chain of a glycoprotein, or directly to the protein moiety. The sugar chain of a glycoprotein may have various structures including type I sugar chain, type II sugar chain, mother nucleus sugar chain, and may be O-linked type and N-linked type.

The fucosylated glycolipid in the present invention includes any glycolipid comprising fucose in its sugar moiety, and examples include, but are not limited to, fucosyl GM1. The sugar chain of a glycolipid may have various structures including type I sugar chain, type II sugar chain, and mother nucleus sugar chain.

In one embodiment of the present invention, the fucosylated molecule-producing cell exhibits increased production of fucosylated molecules, compared to normal cells. Here, normal cells refer to, for example, when the subject cell is a tumor cell, the cells of the same type which have not been subjected to tumorigenic transformation; when the subject cell is a cell at a site of inflammation, then refer to the cells of the same type before occurrence of the inflammation or at a site without inflammation. The amount of production of fucosylated molecules can be appropriately measured using, for example, without limitation, an antibody or lectin that recognizes the above carbohydrate antigen. In one embodiment of the present invention, the amount of fucosylated molecules produced by the fucosylated molecule-producing cell is 2 times or more, preferably 5 times or more, more preferably 10 times or more, furthermore preferably 20 times or more, and particularly preferably 50 times or more than that by a normal cell. In another embodiment, the amount of fucosylated molecules produced by the fucosylated molecule-producing cell is larger than that by the cell lines MIAPaCa, PANC-1, KP4, PK45H, HT-29, HCT-15, RBE, OCUG-1, TGBC14TKB, SSP-25, YSCCC, TKKK, HuH-28, MKN45, MKN74, NUGC-4 and/or MOLT-4, and it is equal to or larger than that by the cell lines PK59, ASPC1, SW1116, LS174T, COLO205, LS180, HuCCT1, JR-St, HSC-39, NCI-N87 and/or HL-60.

In another embodiment of the present invention, the fucosylated molecule-producing cell has a fucose binding mechanism. The fucose binding mechanism refers to a mechanism possessed by the cell, with which the cell selectively binds to and/or takes up fucose, and its examples includes, without limitation, cell components such as receptors and transporters. Presence/absence of a fucose binding mechanism can be determined by investigating the binding amount of fucose detectably labeled with radiolabels, etc., to cells to be examined, as well as its binding constants (see Example 3). For instance, the cell having a fucose binding mechanism exhibits, when measured using the method of Example 3 mentioned below, a binding constant Kd of 25 nM or more, preferably 28 nM or more, more preferably 30 nM or more, and yet more preferably 34 nM or more; and bmax of 5 pmol/10⁶ cells or more, preferably 7.5 pmol/10⁶ cells or more, more preferably 10 pmol/10⁶ cells or more, and yet more preferably 11 pmol/10⁶ cells or more.

In another embodiment of the present invention, the fucosylated molecule-producing cell expresses a fucosyltransferase. The fucosyltransferase is not particularly limited as long as it transfers fucose from a fucose donor to a fucose acceptor (e.g., sugar chain, polypeptide, lipid, etc.), and it includes, for example, FUT1, FUT2, FUT3, FUT4, FUT5, FUT6, FUT7, FUT8, FUT9, FUT10, FUT11, POFUT1, POFUT2, etc., known in the art. In one embodiment of the present invention, the fucosyltransferase is selected from the group consisting of FUT1, FUT2, FUT3, and FUT4. In another embodiment of the present invention, the fucosyltransferase is selected from the group consisting of FUT1, FUT2, FUT4, FUT5, FUT6, and FUT8. In yet another embodiment of the present invention, the fucosyltransferase is selected from the group consisting of FUT1, FUT2, FUT3, FUT4, FUT5, FUT6, and FUT8. In one embodiment of the present invention, the fucosyltransferase is preferably the one that can transfer fucose via α1,4 linkage, and examples of such fucosyltransferase include FUT3, etc. In one embodiment of the present invention, preferable fucosyltransferase is FUT3 and/or FUT6 that have a strong association with production of CA19-9. In one embodiment of the present invention, the fucosyltransferase is preferably the one that can bind fucose to polypeptide, and examples of such fucosyltransferase include POFUT1, POFUT2, etc. In addition, in one embodiment of the present invention, preferable fucosyltransferase is POFUT1 that has a strong association with fucosylation of Notch-1.

The fucosyltransferase may be expressed during a series of protein expression processes from transcription of genes to maturation of proteins; its expression can be detected at a genetic level and/or protein level. Specifically, in the genetic level, expression can be detected by any known gene expression analysis method including northern blotting, southern blotting, DNA microarray analysis, RNase protection assay, PCR method such as RT-PCR and real-time PCR, etc., in situ hybridization method, and in vitro transcription method, etc.; in the protein level, it can be detected by any known protein detection method including immunoprecipitation, western blotting, mass spectrometry, EIA, ELISA, RIA, immunohistochemical method and immunocytochemical method, etc. In one embodiment of the present invention, the amount of expression of fucosyltransferase by the fucosylated molecule-producing cell is 2 times or more, preferably 5 times or more, more preferably 10 times or more, yet more preferably 20 times or more, and particularly preferably 50 times or more than that by a normal cell. In addition, in one embodiment of the present invention, the amount of expression of fucosyltransferase by the fucosylated molecule-producing cell is larger than that by the cell lines MIAPaCa, PANC-1, KP4, PK45H, HT-29, HCT-15, RBE, OCUG-1, TGBC14TKB, SSP-25, YSCCC, TKKK, HuH-28, MKN45, MKN74, NUGC-4 and/or MOLT-4, and it is equal to or larger than that by PK59, ASPC, SW1116, LS174T, COLO205, LS180, HuCCT1, JR-St, HSC-39, NCI-N87 and/or HL-60.

Another aspect of the present invention relates to a substance delivery carrier for cells having a fucose binding mechanism, which comprises fucose. This carrier targets the fucose binding mechanism. Therefore, one embodiment of the present invention relates to a carrier that is targeted by fucose at cells having a fucose binding mechanism. Details of the fucose binding mechanism are as mentioned above. This carrier may comprise an effective amount of fucose for the targeting of cells having a fucose binding mechanism. Accordingly, one embodiment of the present invention relates to a carrier that is targeted at cells having a fucose-binding mechanism, which comprises an effective amount of fucose for the targeting of said cells. Furthermore, this carrier can be used for delivering a substance to cells having a fucose binding mechanism. Accordingly, one embodiment of the present invention relates to a carrier for delivering a substance to cells having a fucose binding mechanism, which comprises fucose. Furthermore, since the fucose binding mechanism is associated with the amount of production of fucosylated molecule and the amount of expression of fucosyltransferase, these can be used as an index of having a fucose binding mechanism. Therefore, in one embodiment of the present invention, the cell having a fucose binding mechanism produces a fucosylated molecule. In addition, in another embodiment of the present invention, the cell having a fucose binding mechanism expresses a fucosyltransferase. Details regarding the production of fucosylated molecule and expression of fucosyltransferase are as described above.

A further aspect of the present invention relates to a substance delivery carrier for cells expressing a fucosyltransferase, which comprises fucose. Said carrier may comprise an effective amount of fucose for the targeting of fucosyltransferase-expressing cells. Accordingly, one embodiment of the present invention relates to a carrier that is targeted at fucosyltransferase-expressing cells, which comprises an effective amount of fucose for the targeting of said cells. Moreover, said carrier can be used for delivering a substance to fucosyltransferase-expressing cells. Accordingly, one embodiment of the present invention relates to a carrier for delivering a substance to fucosyltransferase-expressing cells, which comprises fucose. Details regarding the expression of fucosyltransferase are as described above. Since the expression of fucosyltransferase is associated with the production of fucosylated molecule and presence of fucose binding mechanism, these can be used as an index of expression of fucosyltransferase. Accordingly, in one embodiment of the present invention, the fucosyltransferase-expressing cell produces a fucosylated molecule. Also, in another embodiment of the present invention, the fucosyltransferase-expressing cell has a fucose binding mechanism. Details regarding the production of fucosylated molecule and presence of fucose binding mechanism are as described above.

As used herein, “targeting” means that, compared to non-targeted substances, a substance such as a drug or carrier is delivered to a specific target, for example, specific cells and tissues (in the present invention, fucosylated molecule-producing cells and/or cells having a fucose-binding mechanism and/or fucosyltransferase-expressing cells (hereinafter, only fucosylated molecule-producing cells are mentioned as their representative), as well as tissues comprising said cells), more rapidly, efficiently and/or in a larger amount than to non-targeted cells and tissues, namely, such a substance is specifically delivered to the target; a targeting agent means a substance that, when it is bound to or reacted with another substance, it can make this another substance to be targeted in such a manner. Therefore, the fucose in the present invention functions as a targeting agent. In addition, target specificity means a degree of rapidness, efficiency, and/or amount at which a targeted substance such as a drug or carrier is delivered to a target cell, i.e., fucosylated molecule-producing cell; when target specificity is high, then the targeted substance is delivered to the target cell more efficiently, while its delivery to non-target cells is suppressed.

The fucose contained in the carrier of the present invention is not particularly limited as long as it facilitates the delivery of a substance to fucosylated molecule-producing cells, and examples include L-fucose, D-fucose, a sugar chain comprising L-fucose and/or D-fucose, for example, a sugar chain comprising L-fucose and/or D-fucose at its side chain, or a sugar chain comprising L-fucose and/or D-fucose at its non-reducing terminal, and a polypeptide or lipid to which L-fucose and/or D-fucose is bound.

The carrier of the invention may be composed of these kinds of fucose themselves, or may be composed by binding or including fucose to a carrier constituent other than fucose. Accordingly, the carrier of the invention may comprise a carrier constituent other than fucose. In this case, the relationship between fucose and a carrier constituent other than fucose is not particularly limited as long as the fucose forms a complex with a structure formed by the carrier constituent other than fucose, in a manner that the fucose can function as a targeting agent, and it includes those wherein the fucose binds to the structure formed by the carrier constituent other than fucose directly, or via an intervening chemical element such as a linker or a spacer, in a manner that the fucose can be in contact with a target cell element. As examples of the carrier constituent other than fucose, without limitation, any components known in the fields of medicine and pharmacology may be used; however, those that can include fucose or that can bind to fucose are preferable.

Examples of such constituent include lipids, for example, phospholipid such as glycerophospholipid, sphingolipid such as sphingomyelin, sterol such as cholesterol, vegetable oil such as soybean oil and poppy seed oil, mineral oil, lecithins such as egg yolk lecithin, polyethylene glycol, PEG:polymer carrier, etc., but they are not limited thereto. Of these, those which can constitute a liposome, for example, natural phospholipid such as lecithin, semi-synthetic phospholipid such as dimyristoyl phosphatidylcholine (DMPC), dipalmitoylphosphatidylcholine (DPPC) and distearoylphosphatidylcholine (DSPC), as well as dioleyl phosphatidylethanolamine (DOPE), dilauroyl phosphatidylcholine (DLPC), and cholesterol are preferable.

Particularly preferable constituents include those which can avoid being trapped by the reticuloendothelial system, for example, cationic lipids such as N-(α-trimethylammonioacetyl)-didodecyl-D-glutamate chloride (TMAG), N,N′,N″,N′″-tetramethyl-N,N′,N″,N′″-tetrapalmityl spermine (TMTPS), 2,3-dioleyloxy-N-[2(sperminecarboxamide)ethyl]-N,N-dimethyl-1-propanaminium trifluoroacetate (DOSPA), N-[1-(2,3-dioleyloxy)propyl]-N,N,N-trimethylammonium chloride (DOTMA), dioctadecyl dimethyl ammonium chloride (DODAC), didodecyl ammonium bromide (DDAB), 1,2-dioleyloxy-3-trimethylammoniopropane (DOTAP), 3β-[N-(N′,N′-dimethylaminoethane)carbamoyl]cholesterol (DC-Chol), 1,2-dimyristoyloxypropyl-3-dimethylhydroxyethyl ammonium (DMRIE), O,O′-ditetradecanoyl-N-(α-trimethylammonioacetyl)diethanolamine chloride (DC-6-14), etc.

Binding or inclusion of fucose in the carrier of the invention is also possible by binding or inclusion of fucose to other constituents of the carrier by means of chemical and/or physical method. Examples of the method to bind fucose to a carrier include, but are not limited to: a method wherein a liposome is treated to have a hydrophilic property using tris(hydroxyalkyl)aminoalkane, to which a linker protein, for example a protein derived from living organisms such as human serum albumin (HSA) and bovine serum albumin (BSA), is bound, and a sugar chain is bound to the linker protein (WO2007/091661, Hirai et al., Biochem Biophys Res Commun. 2007; 353(3): 553-8, Hirai et al., Int J Pharm. 2010; 391(1-2): 274-83), a method to prepare liposomes using a sugar-added cholesterol derivative (Patent Literature 1), a method wherein a sugar is added to poly-L-lysine (Negre et al., Supra), a method wherein a liposome is prepared using a glycolipid, or a method wherein p-aminophenyl-D-glycoside is covalently bound to phosphatidylethanolamine liposome using glutaraldehyde (Ghosh P et al., Biochim. Biophys Acta. 1980; 632(4): 562-72), a method wherein a liposome is prepared using cholesten-5-yloxy-N-(4-((1-imino-2-β-D-thioglycosylethyl)amino)butyl) formamide (Non-patent Literature 1), and the like. Alternatively, binding or inclusion of fucose to the carrier of the invention is possible by mixing fucose with other constituents of the carrier at the time of preparing the carrier.

The amount of fucose to be bound to or included in the carrier of the invention may be, without limitation, expressed by the weight ratio in the constituents of the carrier, 0.01%-100%, preferably 0.2%-20%, and more preferably 1-5%. Binding or inclusion of fucose to/in the carrier of the invention may be carried out before a drug, etc. is supported by the carrier, or by simultaneously mixing the carrier, fucose, etc. and a drug, etc., or by mixing the carrier which has already supported a drug, etc. with fucose, etc. Accordingly, the present invention also relates to a process for preparing a formulation specific to fucosylated molecule-producing cells, which includes a step of binding fucose to any existing drug-bound carrier or drug-encapsulated carrier, for example liposomal formulations such as DaunoXome®, Doxil, Caelyx®, Myocet®, etc.

The form of the carrier of the invention may be any form, as long as it can deliver a desired substance or matter to a target cell, and examples include, but are not limited to, polymer micelle, liposome, emulsion, microsphere, nanosphere, polymer matrix, and lipoplex. In the present invention, from the viewpoints of level of delivery efficiency, range of substances being delivered, easiness of formulation and the like, a liposomal form or lipoplex form is preferable, and a cationic liposome comprising a cationic lipid is particularly preferable. When the carrier is in a liposomal form, the molar ratio of the fucose to the liposome-constitutive lipid is preferably from 8:1 to 1:8, more preferably from 4:1 to 1:4, yet more preferably from 2:1 to 1:2, and in particular, it is 1:1. In another embodiment, the concentration of the fucose in a carrier suspension is 5-500 μg/ml, preferably 10-250 μg/ml, more preferably 20-200 μg/ml, and furthermore preferably 25-100 μg/ml.

In the carrier of the present invention, as long as the fucose contained therein is present in a manner that it can function as a targeting molecule, the carrier may comprise a substance being transported in its interior, or the carrier may be attached to the external of a substance being transported, or the carrier may be mixed with a substance being transported. Here, “function as a targeting molecule” means that the carrier comprising fucose reaches and/or is taken up by the target cell more rapidly and/or in an amount larger than that in the carrier without fucose; this can be easily confirmed, e.g., by adding to a cell culture a carrier to which a label is attached or that comprises a label, and by analyzing the site of the label after a certain period of time. Structurally, when the fucose is at least partially exposed outside of the formulation comprising the carrier, or the fucose is present in a form that it can be recognized by a target cell element, at the latest before reaching a target cell, then, the above requirements can be satisfied.

The substance or matter that is delivered by the carrier of the invention is not particularly limited, and it desirably has a size with which it can physically move inside the body from the administration site to the site of lesion where a target cell is present. Accordingly, the carrier of the invention can transport not only substances such as atoms, molecules, compounds, proteins, and nucleic acids, etc., but also matters such as vectors, virus particles, cells, drug-release systems composed of one or more elements, micromachines, etc. The above substances or matters preferably have a characteristic to influence target cells and/or their periphery, and they include, for example, those which can label target cells, or can control (for example, enhance or suppress) the activity or growth of target cells and/or cells present in their periphery.

Therefore, in one embodiment of the present invention, the substance delivered by the carrier is “a drug controlling the activity or growth of fucosylated molecule-producing cells”. Here, the activity of fucosylated molecule-producing cells refers to various activities such as secretion, intake and migration exhibited by said cells; in the case of tumor cells for example, it refers to activities involved in the onset, progression, recurrence and/or metastasis of tumors, and appearance and worsening of symptoms such as cachexia. Such activities include, but are not limited to, production and secretion of parathyroid hormone-related protein (PTHrP) and immunosuppressive acidic protein (IAP).

Therefore, the drug that controls activity or growth of fucosylated molecule-producing cells may be any drug that directly or indirectly suppresses physical, chemical and/or physiological actions, etc. of fucosylated molecule-producing cells related to the onset, progression and/or recurrence of diseases related to fucosylated molecule-producing cells. For example, in the case of tumor cells, such drugs include, without limitation, drugs that inhibit activity or production of the above physiologically active substance, for example an antibody and an antibody fragment that neutralizes the physiologically active substance, a substance that suppresses expression of the physiologically active substance such as siRNA, ribozyme and antisense nucleic acid (including RNA, DNA, PNA and a complex thereof), or a substance that has a dominant negative effect such as a dominant negative mutant, etc., or a vector expressing them, a cell activation inhibitor such as sodium channel inhibitor, cell growth inhibitors, such as an alkylating agent (e.g., ifosfamide, nimustine, cyclophosphamide, dacarbazine, melphalan, ranimustine etc.), an antitumor antibiotic (e.g., idarubicin, epirubicin, daunorubicin, doxorubicin, pirarubicin, bleomycin, peplomycin, mitoxantrone, mitomycin C, etc.), and an antimetabolite (e.g., gemcitabine, enocitabine, cytarabine, tegafur uracil, tegafur gimeracil oteracil potassium formulation, doxifluridine, hydroxycarbamide, fluorouracil, methotrexate, mercaptopurine, etc.); as well as apoptosis-inducing agents, such as compound 861 and gliotoxin. Furthermore, “the drug that controls activity or growth of fucosylated molecule-producing cells” in the present invention may be any drug that directly or indirectly accelerates physical, chemical and/or physiological actions, etc. of fucosylated molecule-producing cells directly or indirectly related to the suppression of the onset, progression, and/or recurrence of diseases related to fucosylated molecule-producing cells.

Examples of the drug that controls activity or growth of fucosylated molecule-producing cells also include substances that suppress production of fucosylated molecules, for example, an antibody and an antibody fragment that inhibit functions of fucosyltransferase, a substance that suppresses express ion of fucosyltransferase such as RNAi molecules (e.g., siRNA, shRNA, ddRNA, miRNA, piRNA, rasiRNA, etc.), ribozyme and antisense nucleic acids (including RNA, DNA, PNA, and a complex thereof), or a substance that has a dominant negative effect such as a dominant negative mutant, etc., or a vector expressing them. Examples of fucosyltransferase include FUT1, FUT2, FUT3, FUT4, FUT5, FUT6, FUT7, and FUT8, etc., and examples of siRNA sequences corresponding thereof include those listed in Table 2 below. Other examples of fucosyltransferase include FUT9, FUT10, FUT11, POFUT1 and POFUT2, etc. In one embodiment of the present invention, fucosyltransferase to be inhibited is selected from the group consisting of FUT1, FUT2, FUT3 and FUT4. In another embodiment of the present invention, fucosyltransferase is selected from the group consisting of FUT1, FUT2, FUT4, FUT5, FUT6 and FUT8. In yet another embodiment of the present invention, fucosyltransferase is selected from the group consisting of FUT1, FUT2, FUT3, FUT4, FUT5, FUT6 and FUT8. In addition, in one embodiment of the present invention, fucosyltransferase to be inhibited is preferably the one that can transfer fucose via α1,4 linkage, and examples of such fucosyltransferase include FUT3, etc. In one embodiment of the present invention, fucosyltransferase to be inhibited is FUT3 and/or FUT6 that have a strong association with production of CA19-9. In another embodiment of the present invention, fucosyltransferase to be inhibited is preferably the one that can bind fucose to polypeptide, and examples of such fucosyltransferase include POFUT1, POFUT2, etc. In addition, in one embodiment of the present invention, fucosyltransferase to be inhibited is POFUT1 that has a strong association with fucosylation of Notch-1.

The substance being delivered by the carrier of the present invention also includes a drug that treats a disease related to fucosylated molecule-producing cells. In the present specification, the disease related to fucosylated molecule-producing cells includes not only a disease caused by fucosylated molecule-producing cells, but also a disease affecting said cells, and examples include, without limitation, neoplastic diseases including tumors such as pancreatic tumor, biliary system tumor, liver tumor, digestive tract tumor, brain tumor, lung tumor, bone and soft tissue tumor, hematopoietic organ tumor, more specifically, for example, pancreatic cancer, biliary system cancer, liver cancer, stomach cancer, esophageal cancer, colorectal cancer, and further, breast cancer, lung cancer, endometrial cancer, prostate cancer, leukemia, lymphoma, etc., as well as inflammatory diseases such as pancreatitis, cirrhosis, hepatitis, etc. In addition, because Notch is activated in endothelial cells, fibroblast cells and keratinocytes which are involved in wound healing (Chigurupati et al., PLoS One. 2007 Nov. 14; 2(11): e1167), the disease related to fucosylated molecule-producing cells also includes wound.

Accordingly, examples of the substance being delivered by the carrier of the present invention include antitumor agents that suppress the onset, progression and/or recurrence of neoplastic diseases, for example, without limitation, alkylating agents such as ifosfamide, nimustine (e.g., nimustine hydrochloride), cyclophosphamide, dacarbazine, melphalan, ranimustine, etc., antimetabolites such as gemcitabine (e.g., gemcitabine hydrochloride), enocitabine, cytarabine ocfosfate, cytarabine preparation, tegafur uracil, tegafur gimeracil oteracil potassium formulation (e.g., TS-1), doxifluridine, hydroxycarbamide, fluorouracil, methotrexate, mercaptopurine, etc., antitumor antibiotics such as idarubicin (e.g., idarubicin hydrochloride), epirubicin (e.g., epirubicin hydrochloride), daunorubicin (e.g., daunorubicin hydrochloride, daunorubicin citrate), doxorubicin (e.g., doxorubicin hydrochloride), pirarubicin (e.g., pirarubicin hydrochloride), bleomycin (e.g., bleomycin hydrochloride), peplomycin (e.g., peplomycin sulfate), mitoxantrone (e.g., mitoxantrone hydrochloride), mitomycin C, etc., alkaloids such as etoposide, irinotecan (e.g., irinotecan hydrochloride), vinorelbine (e.g., vinorelbine tartrate), docetaxel (e.g., docetaxel hydrate), paclitaxel, vincristine (e.g., vincristine sulfate), vindesine (e.g., vindesine sulfate), vinblastine (e.g., vinblastine sulfate), etc., hormone therapy agents such as anastrozole, tamoxifen (e.g., tamoxifen citrate), toremifene (e.g., toremifene citrate), bicalutamide, flutamide, estramustine (e.g., estramustine phosphate), etc., platinum complexes such as carboplatin, cisplatin (CDDP), nedaplatin, etc., angiogenesis inhibitors such as thalidomide, Neovastat, bevacizumab, etc., and L-asparaginase and the like.

Examples of the substance being delivered by the carrier of the present invention may further include, but are not limited to, anti-inflammatory agents that suppress the onset, progression and/or recurrence of inflammatory diseases, for example, steroidal anti-inflammatory agents (prednisolone, beclomethasone, betamethasone, fluticasone, dexamethasone, hydrocortisone, etc.) and non-steroidal anti-inflammatory agents (acetylsalicylic acid, loxoprofen, acetaminophen, ketoprofen, tiaprofenic acid, suprofen, tolmetin, carprofen, benoxaprofen, piroxicam, benzydamine, naproxen, diclofenac, ibuprofen, diflunisal, azapropazone, etc.), substances that suppress expression of inflammatory cytokines such as siRNA molecules (e.g., siRNA, shRNA, ddRNA, miRNA, piRNA, rasiRNA, etc.) and antisense nucleic acids, and/or drugs that suppress action of inflammatory cytokines, such as antibodies against inflammatory cytokines, receptor antagonists of inflammatory cytokines, etc.

The carrier of the present invention preferably delivers a substance being delivered to the interior of a target cell; however, depending on the situation, in some cases delivery to the periphery of a target cell may be preferred. For example, substances that suppress expression of inflammatory cytokines such as siRNA molecules and antisense nucleic acids can be delivered also to inflammatory cytokine-producing cells that do not produce fucosylated molecules, thereby enabling more effective treatment of diseases related to fucosylated molecule-producing cells such as pancreatitis and hepatitis.

The substance or matter being delivered by the carrier of the present invention may be or may not be labeled. By means of labeling, success/failure of transportation, and increase/decrease of target cells can be monitored, and it is particularly useful at investigation and research levels. In the present specification, a label refers to any substance, wherein, the substance itself or a matter to which the substance is attached can be detected directly or indirectly. A label may be selected from substances known by those skilled in the art, for example, gas or substances that generate gas under physiological conditions, any radioisotopes, magnetic materials, nuclear magnetic resonance elements (e.g., hydrogen, phosphorus, sodium, fluorine, etc.), substances that affect relaxation time of nuclear magnetic resonance elements (e.g. metal atom or compound comprising thereof), substances that binds to a labeling substance (e.g., antibody), fluorescent materials, fluorophores, chemiluminescent substances, enzymes, biotin or its derivatives, avidin or its derivatives.

In the present specification, a label may be a detectable label, which includes any labels that can be detected by any existing detection means. Examples of detection method include, but are not limited to, naked eye, optical examination apparatus (e.g., optical microscope, fluorescence microscope, phase contrast microscope, in vivo imaging apparatus, etc.), X-ray apparatus (e.g., plain X-ray apparatus, computed tomography (CT) apparatus, etc.), magnetic resonance imaging (MRI) apparatus, nuclear medicine apparatus (e.g., scintigraphic apparatus, positron emission tomography (PET) apparatus, single photon emission computed tomography (SPECT) apparatus, etc.), ultrasonographic apparatus and thermographic apparatus, etc. Labels suitable for each detection means are known to a person skilled in the art, and described, for example, in Lecchi et al., Q J Nucl Med Mol Imaging. 2007; 51(2): 111-26.

Examples of labels suitable for detection by naked eye and optical examination apparatus include various fluorescent labels and luminescent labels.

Specific fluorescent labels which may be used include, but are not limited to, Cy™ series (e.g., Cy™ 2, 3, 5, 5.5, 7, etc.), DyLight™ series (e.g., DyLight™ 405, 488, 549, 594, 633, 649, 680, 750, 800, etc.), Alexa Fluor® series (e.g., Alexa Fluor® 405, 488, 549, 594, 633, 647, 680, 750, etc.), HiLyte Fluor™ series (e.g., HiLyte Fluor™ 488, 555, 647, 680, 750, etc.), ATTO series (e.g., ATTO 488, 550, 633, 647N, 655, 740, etc.), FAM, FITC, Texas-Red, GFP, RFP, and Qdot. Fluorescent labels suitable for in vivo imaging are, for example, those emit a fluorescence of wavelength that are highly transmissive through living body and less susceptible to autonomous fluorescence, such as a fluorescence of near-infrared wavelength, or those exhibit strong fluorescent intensity. Such fluorescent labels include, but are not limited to, Cy™ series, DyLight™ series, Alexa Fluor® series, HiLyte Fluor™ series, ATTO series, Texas-Red, GFP, RFP, Qdot and derivatives thereof.

Specific luminescent labels which may be used include, but are not limited to, for example, luminol, luciferin, lucigenin and aequorin, etc.

Suitable labels for detection by X-ray apparatus include, for example, various contrast agents. Specific contrast agents which may be used include, but are not limited to, iodine atoms, iodine ions and iodine-containing compounds, etc.

Suitable labels for detection by MRI apparatus include, for example, nuclear magnetic resonance elements and substances that affect relaxation time of nuclear magnetic resonance elements. Examples of the nuclear magnetic resonance elements include hydrogen, phosphorus, sodium, fluorine, etc. Examples of the substances that affect relaxation time of nuclear magnetic resonance elements include, but are not limited to, various metal atoms, or a compound comprising said metal atom(s), for example, complexes of said metal atom (s). Specific examples that may be used include, but are not limited to, gadolinium(III) (Gd(III)), yttrium-88 (⁸⁸Y), indium-111 (¹¹¹In), complexes of such metal atom(s) and a ligand such as diethylenetriaminepentaacetic acid (DTPA), tetraazacyclododecane-1,4,7,10-tetraacetic acid (DOTA), (1,2-ethanediyldinitrilo)tetraacetic acid (EDTA), ethylenediamine, 2,2′-bipyridine (bipy), 1,10-phenanthroline (phen), 1,2-bis(diphenylphosphino)ethane (DPPE), 2,4-pentanedione (acac), and oxalate (ox), as well as super-paramagnetic iron oxide (SPIO) and manganese oxide (MnO).

Suitable labels for detection by nuclear medicine apparatus include, for example, various radioisotopes, and compounds comprising said radioisotope (s), such as complexes of said radioisotope(s). Radioisotopes which may be used include, but are not limited to, e.g., technetium-99m (^{33m}Tc), indium-111 (¹¹¹In), iodine-123 (¹²³I), iodine-124 (¹²⁴I), iodine-125 (¹²⁵I), iodine-131 (¹³¹I), thallium-201 (²⁰¹I), carbon-11 (¹¹C), nitrogen-13 (¹³N), oxygen-15 (¹⁵O), fluorine-18 (¹⁸F), copper-64 (⁶⁴Cu), gallium-67 (⁶⁷Ga), krypton-81m (^{81m}Kr), xenon-133 (¹³³Xe), strontium-89 (⁸⁹Sr) and yttrium-90 (⁹⁰Y). Compounds comprising a radioisotope include, but are not limited to, e.g., ¹²³I-IMP, ^{99m}Tc-HMPAO, ^{99m}Tc-ECD, ^{99m}Tc-MDP, ^{99m}Tc-tetrofosmin, ^{99m}Tc-MIBI, ^{99m}TcO₄—, ^{99m}Tc-MAA, ^{99m}Tc-MAG3, ^{99m}Tc-DTPA, ^{99m}Tc-DMSA, ¹⁸F-FDG1, etc.

Suitable labels for detection by ultrasonographic apparatus which may be used include, but are not limited to, bio-acceptable gases or substances that generate gas under physiological conditions, fatty acids, or substances comprising thereof. Examples of the gas include, but are not limited to, air, noble gas, nitrogen, N₂O, oxygen, carbon dioxide, hydrogen, inert noble gas (e.g., helium, argon, xenon or krypton), sulfurfluoride (e.g., sulfurhexafluoride, disulfur decafluoride, trifluoromethyl sulfur pentafluoride), seleniumhexafluoride, silane halides (e.g., tetramethyl silane), low-molecular-weight hydrocarbon (e.g., C₁₋₇ alkane (methane, ethane, propane, butane, pentane, etc.), cycloalkane (cyclobutane, cyclopentane, etc.), alkene (ethylene, propene, butene, etc.)), fluorine-containing gas, ammonia, etc.; examples of substances that generate gas under physiological conditions include, but are not limited to, dodecafluoropentane (DDFP), perfluorocarbon vaporized under physiological conditions (JP A 2010-138137); examples of substances comprising the above substances include nanoparticles and liposomes. Examples of fluorine-containing gas include, but are not limited to, halogenated hydrocarbon gas (e.g., bromochlorodifluoromethane, chlorodifluoromethane, dichlorodifluoromethane, bromotrifluoromethane, chlorotrifluoromethane, chloropentafluoroethane, dichlorotetrafluoroethane, perfluorocarbon), fluorinated ketone (e.g., perfluoroacetone), fluorinated ether (e.g., perfluoro-diethyl ether).

Examples of perfluorocarbon include, but are not limited to, perfluoroalkane (e.g., perfluoromethane, perfluoroethane, perfluoropropane, perfluorobutane, perfluoro-n-butane, perfluoropentane, perfluorohexane, perfluoroheptane), perfluoroalkene (e.g., perfluoropropene, perfluorobutene (e.g., perfluorobut-2-ene), perfluorobutadiene), perfluoroalkyne (e.g., perfluorobut-2-yne), perfluorocycloalkane (e.g., perfluorocyclobutane, perfluoromethylcyclobutane, perfluorodimethylcyclobutane, perfluorotrimethylcyclobutane, perfluorocyclopentane, perfluoromethylcyclopentane, perfluorodimethylcyclopentane, perfluorocyclohexane, perfluoromethylcyclohexane, perfluorocycloheptane), etc.

As suitable labels for detection by ultrasonographic apparatus, those already commercially available may also be used. Examples of commercially available labels for ultrasonographic detection include, but are not limited to, those of the first generation such as Albunex (Mall inckrodt), Echovist (SHU 454, Schering), Levovist (SHU 508, Schering), Myomap (Quadrant), Quantison (Quadrant), Sonavist (Schering), Sonazoid (GE Healthcare), etc., those of the second generation such as Definity/luminity (Bristol-Myers Squibb Medical Imaging), Imagent-imavist (Alliance), Optison (GE healthcare), biSphere/cardiosphere (POINT Biomedical), SonoVue (BR1, Bracco), AI700/imagify (Acusphere), etc., those of the third generation such as Echogen (Sonus pharmaceuticals), etc. (Reddy et al., World J Gastroenterol. 2011 Jan. 7; 17(1):42-8). In addition, suitable labels for detection by ultrasonographic apparatus other than those mentioned above are described in JPA 5-194278, JP A 8-310971, JPA 8-151335, JP A 2002-308802, WO2004/069284, WO 2005/120587, etc.

In the present invention, “for fucosylated molecule-producing cell” means that it is suitable to use a fucosylated molecule-producing cell as a target; this includes, e.g., that a substance can be delivered to a fucosylated molecule-producing cell more rapidly, with higher efficiency and/or in a larger amount than to a fucosylated molecule-non-producing cell. For instance, the carrier of the invention can deliver a substance to a fucosylated molecule-producing cell, at a rate and/or efficiency of 1.1 times or more, 1.2 times or more, 1.3 times or more, 1.5 times or more, 2 times or more, and furthermore, 3 times or more than to a fucosylated molecule-non-producing cell.

The present invention also relates to a composition comprising said carrier and said drug that controls the activity or growth of fucosylated molecule-producing cells (hereinafter, also referred to as drug-containing composition), and to the use of said carrier in the preparation of such compositions. In one embodiment of the present invention, said composition may be those used for controlling the activity or growth of fucosylated molecule-producing cells, or for treating diseases related to fucosylated molecule-producing cells. Moreover, said composition may comprise a drug that controls the activity or growth of fucosylated molecule-producing cells in an effective amount for controlling the activity or growth of fucosylated molecule-producing cells, or for treating diseases related to fucosylated molecule-producing cells. Here, the effective amount is, in the latter case, an amount to suppress the onset or recurrence of, to alleviate the symptoms of, or to delay or halt the progression of said diseases, and it is preferably an amount to prevent the onset or recurrence of, and to cure said diseases. In addition, an amount that does not cause an adverse effect exceeding the benefit from the administration is preferred. Such an amount can be appropriately determined by in vitro tests using culture cells, and by examination using a model animal such as a mouse, rat, dog or pig, and such examination methods are well known to those skilled in the art. Moreover, the doses of fucose contained in the carrier and of a drug used in the method of the present invention are known to a person skilled in the art, or may be determined as appropriate by the above-mentioned tests, etc.

A drug that controls the activity or growth of fucosylated molecule-producing cells in the composition of the present invention and a disease related to fucosylated molecule-producing cells are as described above in relation to the carrier of the present invention. Accordingly, said composition may comprise a labeled drug. Furthermore, said composition may comprise, in addition to the drug that controls the activity or growth of fucosylated molecule-producing cells, a label, and other drugs, for example, the above-mentioned drugs, etc. that treat diseases related to fucosylated molecule-producing cells.

The present invention also relates to a composition comprising said carrier and a label (hereinafter, also referred to as a label-containing composition), and to the use of said carrier in the preparation of such compositions. In one embodiment of the present invention, said composition may be those for labeling fucosylated molecule-producing cells or tissues comprising said cells, for detecting fucosylated molecule-producing cells or tissues comprising said cells, for diagnosing, detecting and/or monitoring a disease related to fucosylated molecule-producing cells, for detecting the possibility of a disease related to fucosylated molecule-producing cells, for aiding the diagnosis of a disease related to fucosylated molecule-producing cells, or for evaluating the effects of a treatment of a disease related to fucosylated molecule-producing cells. Fucosylated molecule-producing cells or tissues comprising said cells may be detected by in vivo or in vitro imaging. Accordingly, the above-mentioned composition may be used for in vivo or in vitro imaging of fucosylated molecule-producing cells or tissues comprising said cells. Furthermore, said composition may comprise a label in an effective amount for labeling fucosylated molecule-producing cells or tissues comprising said cells, for detecting fucosylated molecule-producing cells or tissues comprising said cells, for in vivo or in vitro imaging of fucosylated molecule-producing cells or tissues comprising said cells, for diagnosing, detecting and/or monitoring a disease related to fucosylated molecule-producing cells, for detecting the possibility of a disease related to fucosylated molecule-producing cells, for aiding the diagnosis of a disease related to fucosylated molecule-producing cells, or for evaluating the effects of a treatment of a disease related to fucosylated molecule-producing cells.

Here, the effective amount of a label for labeling fucosylated molecule-producing cells or tissues comprising said cells, for detecting fucosylated molecule-producing cells or tissues comprising said cells, for in vivo or in vitro imaging of fucosylated molecule-producing cells or tissues comprising said cells, for diagnosing, detecting and/or monitoring a disease related to fucosylated molecule-producing cells, for detecting the possibility of a disease related to fucosylated molecule-producing cells, for aiding the diagnosis of a disease related to fucosylated molecule-producing cells, or for evaluating the effects of a treatment of a disease related to fucosylated molecule-producing cells may be an amount that is taken into fucosylated molecule-producing cells to a degree that the label can be detected in vivo or in vitro. In addition, an amount that does not cause an adverse effect exceeding the benefit from the administration is preferred. Such an amount can be appropriately determined by in vitro tests using culture cells, and by examination using a model animal such as a mouse, rat, dog or pig, and such examination methods are well known to those skilled in the art.

A label in the label-containing composition and a disease related to fucosylated molecule-producing cells are as described above in relation to the carrier of the present invention. Examples of the tissues comprising fucosylated molecule-producing cells include, but are not limited to, tumor tissues, for example, tissues of pancreatic tumor, biliary system tumor, liver tumor, digestive tract tumor, brain tumor, lung tumor, bone and soft tissue tumor, hematopoietic organ tumor, more specifically, pancreatic cancer, biliary system cancer, liver cancer, stomach cancer, esophageal cancer, colorectal cancer, and further, breast cancer, lung cancer, endometrial cancer, prostate cancer, lymphoma, etc., as well as bone-marrow tissues affected by leukemia, tissues at the site of inflammation in inflammatory diseases such as pancreatitis, cirrhosis, hepatitis, etc., and tissues in the immune system such as the lymphatic system, and dermal tissues affected by wound, etc. Said composition may comprise, in addition to a label, any drug such as the above-described drug that controls the activity or growth of fucosylated molecule-producing cells, and a drug that treats a disease related to fucosylated molecule-producing cells, etc.

In the composition of the present invention, as long as fucose contained in the carrier is present such that it functions as a targeting molecule, the carrier may comprise a substance being delivered in its interior, or the carrier may attach to the exterior of a substance being delivered, or the carrier may be mixed with a substance being delivered. Accordingly, depending on the route of administration and the manner of drug release, the above composition may be covered with an appropriate material, such as enteric coating or a material with timed disintegration, or the composition may be incorporated in an appropriate drug release system.

The composition of the present invention may be administered via various routes including both oral and parenteral routes, and examples include, but are not limited to, oral, intravenous, intramuscular, subcutaneous, topical, rectal, intraarterial, intraportal, intraventricular, transmucosal, transdermal, intranasal, intraperitoneal, intrapulmonary, and intrauterine routes, etc., and the composition may be formulated in a dosage form suitable for each of the administration routes. As such dosage form and formulation method, any known dosage forms and formulation methods may be appropriately adopted (for example, see Hyojun Yakuzaigaku (Standard Phamaceutics), Ed. Yoshiteru Watanabe et al., Nankodo, 2003).

Dosage forms suitable for oral administration include, but are not limited to, powders, granules, tablets, capsules, solutions, suspensions, emulsions, gels, syrups, etc., and dosage forms suitable for parenteral administration include injections such as solution injections, suspension injections, emulsion injections and injections in a form that is prepared at the time of use. Formulations for parenteral administration may be in a form of aqueous or non-aqueous isotonic sterile solution or suspension.

Therefore, the composition of the present invention may be a pharmaceutical composition comprising one or more pharmaceutically acceptable surfactants, carriers, diluents and/or excipients. Pharmaceutically acceptable carriers and diluents, etc. are well known in the field of pharmaceuticals, and are described, for example, Remington's Pharmaceutical Sciences, 18th Ed., Mack Publishing Co., Easton, Pa. (1990), which is incorporated herein by reference in its entirety.

The carrier or the composition of the present invention may be supplied in any forms, and from the viewpoint of preservation stability, preferably it is provided in a form that can be prepared at the time of use, for example, in a form that it can be prepared at a site of clinical practice or its vicinity by a doctor, and/or pharmacist, nurse, or other paramedical staff. In this case, the carrier or the composition of the present invention is provided in one or more containers containing at least one of the essential constituents, and it is prepared before use, for example within 24 hr, preferably within 3 hr, and more preferably just prior to use. Upon preparation, reagents, solvents and formulation tools usually available at a site of preparation can be appropriately used.

Accordingly, the present invention also relates to a kit for preparing the carrier or composition, the kit containing one or more containers that contain fucose, and/or a substance being delivered, and/or a carrier constituent other than fucose, singly or in a combination thereof, and also relates to an essential constituent of the carrier or composition provided in such a kit. The kit of the present invention may further contain, in addition to the above, an instruction regarding preparation method and administration method of the carrier and composition of the invention, for example, an explanatory leaflet, or a recording medium containing information on the method of use such as flexible disk, CD, DVD, blue ray disk, memory card, USB memory, etc. Furthermore, the kit of the present invention may contain all the constituents necessary for completing the carrier or composition of the invention, but it does not necessarily contain all the constituents. Accordingly, the kit of the present invention does not have to contain reagents and solvents usually available at clinical practice sites or experimental facilities, such as sterile water, physiological saline, and glucose solution, etc.

The present invention further relates to a method for controlling the activity or growth of fucosylated molecule-producing cells, or for treating a disease related to fucosylated molecule-producing cells, the method comprising administering an effective amount of said drug-containing composition to a subject in need thereof. A fucosylated molecule-producing cell, activity of fucosylated molecule-producing cells, a disease related to fucosylated molecule-producing cells, and an effective amount are as described above in relation to the carrier or composition of the present invention.

The specific dose of the composition administered in the control or treatment method of the present invention can be determined by taking into account various conditions regarding the subject in need of the control or treatment, such as, severity of symptoms, general health conditions of the subject, age, body weight, gender of the subject, diet, time and frequency of administration, concomitant medicines, response to therapy, and compliance to therapy.

Regarding the administration route, various routes including both oral and parenteral routes are included, for example, oral, intravenous, intramuscular, subcutaneous, topical, rectal, intraarterial, intraportal, intraventricular, transmucosal, transdermal, intranasal, intraperitoneal, intrapulmonary and intrauterine routes, etc.

Frequency of administration differs depending on the nature of the composition used and the above conditions of the subject; examples may include multiple times per day (namely, 2, 3, 4 times, or 5 times or more per day), once a day, once in several days (namely, every 2, 3, 4, 5, 6, 7 days, etc.), several times per week (e.g., 2, 3, or 4 times a week), every one week, and every several weeks (namely, every 2, 3, or 4 weeks).

In the control or treatment method of the present invention, the term “subject” means any individual organism, and is preferably an animal, more preferably a mammal, and furthermore preferably a human individual. In the present invention, the subject may be healthy or may suffer from a certain disease; when the treatment, detection, diagnosis or monitoring of a disease related to fucosylated molecule-producing cells is contemplated, the subject typically means those suffer from said disease, or have a risk of suffering from said disease. Furthermore, when the evaluation of effects of a treatment of a disease related to fucosylated molecule-producing cells is contemplated, the subject typically means those who are receiving a treatment of said disease, or those who are going to receive a treatment.

In addition, the term “treatment” is intended to encompass any kinds of medically acceptable preventive and/or therapeutic intervention with the aim of cure, transient remission or prevention of a disease. For example, the term “treatment” encompasses medically acceptable interventions with various objects, including delay or halt of progression of a disease related to fucosylated sugar chain-producing cells, regression or elimination of lesions, prevention of onset of or prevention of recurrence of said disease.

The present invention further relates to a method that comprises administering an effective amount of said label-containing composition to a subject in need thereof, in order for labeling fucosylated molecule-producing cells or tissues comprising said cells, for detecting in vivo or in vitro fucosylated molecule-producing cells or tissues comprising said cells, for imaging in vivo or in vitro fucosylated molecule-producing cells or tissues comprising said cells, for diagnosing, detecting and/or monitoring a disease related to fucosylated molecule-producing cells, for detecting the possibility of a disease related to fucosylated molecule-producing cells, for aiding the diagnosis of a disease related to fucosylated molecule-producing cells, or for evaluating the effects of a treatment of a disease related to fucosylated molecule-producing cells. Said effective amount may be an amount that is effective for labeling fucosylated molecule-producing cells or tissues comprising said cells, for detecting fucosylated molecule-producing cells or tissues comprising said cells, for imaging in vivo or in vitro fucosylated molecule-producing cells or tissues comprising said cells, for diagnosing, detecting and/or monitoring a disease related to fucosylated molecule-producing cells, for detecting the possibility of a disease related to fucosylated molecule-producing cells, for aiding the diagnosis of a disease related to fucosylated molecule-producing cells, or for evaluating the effects of a treatment of a disease related to fucosylated molecule-producing cells, or an amount effective for detection. A fucosylated molecule-producing cell, activity of fucosylated molecule-producing cells, a disease related to fucosylated molecule-producing cells, and an effective amount, etc. are as described above in relation to the carrier or composition of the present invention.

The specific dose of the composition administered in the methods of detection, imaging, diagnosis, diagnostic aid, monitoring and/or evaluation of the present invention can be determined by taking into account various conditions regarding the subject in need of such act, for example, object of said act, stage of the disease, contents of treatment, general health conditions of the subject, age, body weight, gender of the subject, diet, time and frequency of administration, concomitant medicines, compliance to administration, etc.

Regarding the administration route, various routes including both oral and parenteral routes are included, for example, oral, intravenous, intramuscular, subcutaneous, topical, rectal, intraarterial, intraportal, intraventricular, transmucosal, transdermal, intranasal, intraperitoneal, intrapulmonary and intrauterine routes, etc.

Frequency of administration differs depending on the nature of the composition used and the above conditions of the subject; examples may include multiple times per day (namely, 2, 3, 4 times, or 5 times or more per day), once a day, once in several days (namely, every 2, 3, 4, 5, 6, 7 days, etc.), several times per week (e.g., 2, 3, or 4 times a week), every one week, and every several weeks (namely, every 2, 3, or 4 weeks).

The methods of detection, imaging, diagnosis, monitoring and/or evaluation of the present invention may furthermore contain detecting a label contained in said label-containing composition. A label may be contained in the composition at the time of detection, or may be present separately. Detection of labels may be carried out by any means that can detect the labels, and examples include, but are not limited to, naked eye, optical examination apparatus (e.g., optical microscope, fluorescence microscope, phase contrast microscope, in vivo imaging apparatus, etc.), X-ray apparatus (e.g., plain X-ray apparatus, computed tomography (CT) apparatus, etc.), magnetic resonance imaging (MRI) apparatus, nuclear medicine apparatus (e.g., scintigraphic apparatus, PET apparatus, SPECT apparatus, etc.), ultrasonographic apparatus and thermographic apparatus, etc. Labels suitable for each detection means are known to a person skilled in the art (for example, refer to Lecchi et al., Q J Nucl Med Mol Imaging. 2007; 51(2): 111-26, etc.), and non-limiting examples are already described above.

In one embodiment of the present invention, fucosylated molecule-producing cells are detected (for example, imaged) in vivo. In such detection, any apparatus suitable for in vivo detection can be used, and examples include, but are not limited to, optical examination apparatus (e.g., in vivo imaging apparatus, etc.), X-ray apparatus (e.g., plain X-ray apparatus, computed tomography (CT) apparatus, etc.), magnetic resonance imaging (MRI) apparatus, nuclear medicine apparatus (e.g., scintigraphic apparatus, PET apparatus, SPECT apparatus, etc.), ultrasonographic apparatus and thermographic apparatus, etc. Labels suitable for such detection are also known to a person skilled in the art (for example, refer to Lecchi et al., Q J Nucl Med Mol Imaging. 2007; 51(2): 111-26, etc.).

By detecting (for example, imaging) fucosylated molecule-producing cells in vivo, it is possible to determine the location (for example, organ or part of a body) of fucosylated molecule-producing cells, and the lesion of a disease related to fucosylated molecule-producing cells. Accordingly, the present invention also relates to a method for determining the location of fucosylated molecule-producing cells and/or the lesion of a disease related to fucosylated molecule-producing cells, the method comprising administering an effective amount of said label-containing composition to a subject in need thereof. Such method can contribute to the diagnosis of diseases related to fucosylated molecule-producing cells.

Furthermore, by detecting a label in vitro or in vivo, it is possible to obtain information that contributes to the diagnosis of diseases related to fucosylated molecule-producing cells, such as the number and distribution of fucosylated molecule-producing cells. Therefore, the present invention also relates to a method for aiding the diagnosis of a disease related to fucosylated molecule-producing cells, the method comprising administering an effective amount of said label-containing composition to a subject in need thereof. This method may further comprise providing information that contributes to the diagnosis of diseases related to fucosylated molecule-producing cells, to a medical doctor.

The method for detecting and the method for diagnosing a disease related to fucosylated molecule-producing cells, the method for detecting the possibility of a disease related to fucosylated molecule-producing cells, and the method for aiding the diagnosis of a disease related to fucosylated molecule-producing cells of the present invention may furthermore comprise comparing the detection result of a label in a subject with the detection result of a reference label. The detection result of the reference label may be, for example, a detection result of a label in a subject who is determined not to have a disease related to fucosylated molecule-producing cells (also referred to as “negative detection result”), or may be a detection result of a label in a subject who is determined to have a disease related to fucosylated molecule-producing cells (also referred to as “positive detection result”). Here, for instance, when the detection result of a label in a subject is equal to the negative detection result (for example, there is no significant difference between them), then the subject can be determined to be negative, and when the detection result of a label in a subject significantly exceeds the negative detection result, then the subject can be determined to be positive. In addition, when the detection result of a label in a subject is equal to the positive detection result (for example, there is no

significant difference between them), then the subject can be determined to be positive.

The detection result of a label in the methods of detection, imaging, diagnosis, monitoring and/or evaluation of the present invention may be a signal intensity and/or signal distribution of the detected label.

Here, the signal intensity of a label is meant herein to refer to an intensity or a measurement similar thereto of various signals emitted from the label, such as fluorescent signal, luminescent signal, magnetic signal and radioactive signal, and typically is measured by an appropriate detection means. Specific examples a detection means are already discussed above. The signal intensity may be those obtained from an entire subject or those obtained from a specific site or region of a subject. The signal intensity may also be an average value or an integrated value with regard to the area or the volume of a site to be measured. In case where a signal intensity changes over time, the signal intensity of the present method may be of a specific time point, or may be integrated for a given time period. When the number and activity, etc. of fucosylated molecule-producing cells increase with progression of a disease, then an increase in the signal intensity can be an index of presence or progression of the disease, and conversely, a decrease in the signal intensity can be an index of improvement of the disease.

The signal distribution of a label is meant herein to refer to information on the position of a signal emitted from the label in a subject, and it may be two-dimensional or three-dimensional. By matching the signal distribution with an anatomical relative position of organs or with structural information of tissues such as CT image, MRI image or ultrasound image, it is possible to identify from which tissue the signal is emitted. In case where a signal distribution changes over time, the signal distribution of the present method may be of a specific time point, or may be integrated for a given time period. When a region where fucosylated molecule-producing cells are present expands with progression of a disease, then an expansion in the signal distribution can be an index of presence or progression of the disease, and conversely, a shrinkage in the signal distribution can be an index of improvement of the disease.

In the method of the present invention, it is also possible to evaluate the combination of signal intensity and signal distribution. A simultaneous evaluation of both intensity and position of the signal allows a more accurate determination, as well as provision of more accurate information.

The monitoring method of the present invention may comprise a step of comparing a detection result at a first time point with a detection result at a second time point that is later than the first time point. For instance, when the detection result is an index regarding the number of fucosylated molecule-producing cells (e.g., a signal intensity or signal distribution, etc. of a label taken up by fucosylated molecule-producing cells), and when the index at the second time point is smaller than the index at the first time point, this indicates a decrease in the number of fucosylated molecule-producing cells; when a disease related to fucosylated molecule-producing cells is the one that worsens with growth of fucosylated molecule-producing cells, then this result means that the disease related to fucosylated molecule-producing cells has been improved. For instance, when the signal intensity at the second time point is lower than the signal intensity at the first time point, then the disease can be determined to be improved, and conversely, when the signal intensity at the second time point is higher than the signal intensity at the first time point, then the disease can be determined to be worsened. Furthermore, for instance, when the signal distribution at the second time point is narrower than the signal distribution at the first time point, then the disease can be determined to be improved, and conversely, when the signal distribution at the second time point is broader than the signal distribution at the first time point, then the disease can be determined to be worsened.

The evaluation method of effects of a treatment of the present invention may further comprise a step of comparing a detect ion result of a first time point prior to the treatment with a detection result of a second time point after the treatment, which is later than the first time point, or comparing a detection result of a first time point that is after a first treatment with a detection result of a second time point that is after a second treatment performed after the first treatment. For instance, when the detection result is an index regarding the number of fucosylated molecule-producing cells (e.g., a signal intensity or signal distribution, etc. of a label taken up by fucosylated molecule-producing cells), and when the index at the second time point is smaller than the index at the first time point, this indicates a decrease in the number of fucosylated molecule-producing cells; when a disease related to fucosylated molecule-producing cells is the one that worsens with growth of fucosylated molecule-producing cells, then this result means that the disease related to fucosylated molecule-producing cells has been improved, namely, the effects of the treatment is positive. For instance, when the signal intensity at the second time point is lower than the signal intensity at the first time point, then it can be determined that the disease has improved by the treatment, and therefore the treatment is successful. Conversely, when the signal intensity at the second time point is higher than the signal intensity at the first time point, then it can be determined that the disease has worsened by the treatment, so that the treatment is not so successful or unsuccessful. Furthermore, for instance, when the signal distribution at the second time point is narrower than the signal distribution at the first time point, then it can be determined that the disease has been improved by the treatment, and therefore the treatment is successful, and conversely, when the signal distribution at the second time point is broader than the signal distribution at the first time point, then it can be determined that the disease has been worsened by the treatment, so that the treatment is not so successful or unsuccessful.

The present invention furthermore relates to a method for delivering a substance to fucosylated molecule-producing cells using the above carrier. This method includes, but is not limited to, for example a step for making said carrier to support a substance being delivered, and a step for administering or adding the carrier that supports the substance being delivered to an organism or a medium, such as a culture medium, that comprises fucosylated molecule-producing cells. These steps can be appropriately achieved in accordance with any known method, or any method described herein. The above delivery method may be combined with other delivery method, such as other delivery method targeting at an organ in which fucosylated molecule-producing cells are present. Furthermore, the above method includes an embodiment wherein the method is carried out in vitro, and an embodiment wherein the method is carried out in vivo, e.g., an embodiment wherein fucosylated molecule-producing cells in the body are targeted.

### EXAMPLES

The present invention is described more in detail with reference to the following Examples; however, these examples are intended for exemplification, and they do not limit the scope of the present invention.

Cell culture method used in the Examples of the present invention is shown below. Pancreatic cancer cell lines KP4, PK-59, PK-45H, MIAPaCa2 and PANC-1, biliary tract cancer cell lines HuCCT1, RBE, TGBC24TKB, TGBC14TKB, SSP-25, YSCCC, TKKK and HuH-28, stomach cancer cell lines MKN45, MKN74, NUGC-4 and KATO-III were obtained from RIKEN BioResource Center, pancreatic cancer cell lines AsPC-1 and BxPC-3, colorectal cancer cell lines SW1116, COLO205, HT-29 and HCT-15, stomach cancer cell line NCI-N87, leukemia cell lines HL-60, RPMI8226, KG-1 and MOLT-4 were obtained from American Type Culture Collection (ATCC), colorectal cancer cell line LS174T was obtained from Tohoku University, colorectal cancer cell line LS180 was obtained from DS Pharma, stomach cancer cell line OCUG-1 was obtained from JCRB, stomach cancer cell lines JR-St and HSC-39 were obtained from IBL.

Cells were cultured as follows: BxPC-3, AsPC-1, PANC-1, PK-45H, and PK-59 cells were cultured in RPMI-1640 medium (GIBCO) comprising L-glutamine and 1% penicillin-streptomycin (Invitrogen); KP4 and MIAPaCa2 cells were cultured in 10% FBS-supplemented DMEM (GIBCO) comprising L-glutamine and 1% penicillin-streptomycin (Invitrogen); COLO205, HCT-15, HuCCT1, RBE, SSP-25, YSCCC, NCI-N87, MKN45, MKN74, NUGC-4, KATO-III, HL-60, RPMI8226, KG-1 and MOLT-4 cells were cultured in 10% FBS-supplemented RPMI1640 medium (GIBCO); SW1116 cells were cultured in 10% FBS-supplemented Leibovitz's L-15 medium; LS174T, LS180 and HuH-28 cells were cultured in 10% FBS-supplemented MEM; HT-29 cells were cultured in 10% FBS-supplemented McCoy's 5A medium; OCUG-1, TGBC24TKB, TGBC14TKB and TKKK cells were cultured in 10% FBS-supplemented DMEM (GIBCO); JR-St and HSC-39 cells were cultured in 10% FBS-supplemented TIL medium. Culture conditions of each cell are known to those skilled in the art, and are available from suppliers of the cells.

### Example 1

### Investigation of Tumor Marker Concentration in Supernatant of Various Pancreatic Cancer Cell Cultures

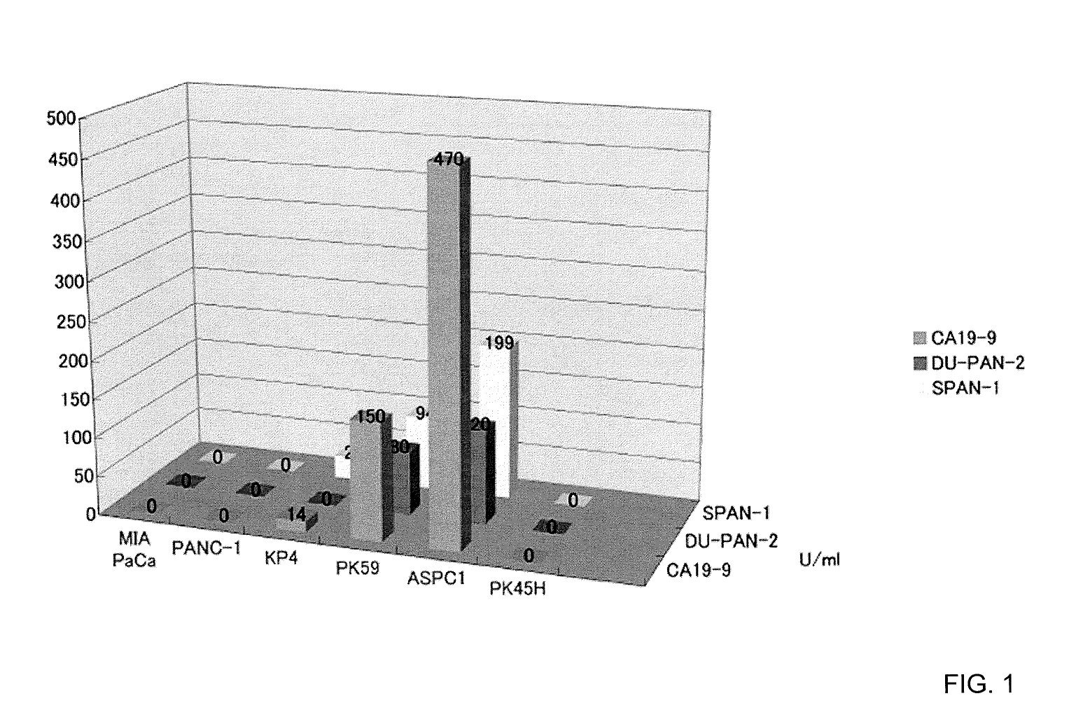

5×10⁶ cells of each pancreatic cancer cell line were seeded in a 25-cm² flask, and cultured with 3 ml of serum-free medium Opti-MEM® for 48 hr. The concentrations of the fucosylated carbohydrate antigen tumor markers CA19-9, Span-1 and Dupan-2 in the supernatant of the cultures were investigated by ELISA method. Results are shown in FIG. 1. Based on the results, PK59 and ASPC-1 were designated to be fucosylated sugar chain high-producing cell lines, and MIAPaCa and PANC-1 to be fucosylated sugar chain low-producing cell lines.

### Example 2

### Expression of Fucosyltransferase in Various Pancreatic Cancer Cell Lines

With respect to each cell line PK59, ASPC-1, MIAPaCa and PANC-1, total RNA was extracted from 1×10⁶ cells and subjected to RT-PCR. Using random hexamer (100 pM) and MMLV (GIBCO), total RNA (1 μg) was reverse-transcribed in accordance with manufacturer's instructions. Primers for each FUT were prepared based on Non-patent Literature 2. [Table 1]

TABLE 1
Primers for FUT1 to FUT7

| Name | Type | Sequence |
|---|---|---|
| FUT1 | Upper | ATGTGGCTCCGGAGCCATCGTCAG |
| | strand | (SEQ ID NO 1) |
| | Lower | AGGATCTCTCAAGTCCGCGTACTC |
| | strand | (SEQ ID NO 2) |
| FUT2 | Upper | CTAGCGAAGATTCAAGCCATGTGG |
| | strand | (SEQ ID NO 3) |
| | Lower | GACGTACTCCCCCGGGATGTG |
| | strand | (SEQ ID NO 4) |
| FUT3 | Upper | ATGGATCCCCTGGGTGCAGCCAAG |
| | strand | (SEQ ID NO 5) |
| | Lower | TCAGGTGAACCAAGCCGCTATGCT |
| | strand | (SEQ ID NO 6) |
| FUT4 | Upper | GTGCCCGAAATTGGGCTCCTGCAC |
| | strand | (SEQ ID NO 7) |
| | Lower | GAAGGAGGTGATGTGGACAGCGTA |
| | strand | (SEQ ID NO 8) |
| FUT5 | Upper | CTTATGGCAGTGGAACCTGTCACC |
| | strand | (SEQ ID NO 9) |
| | Lower | CCAGCCGTAGGGCGTGAAGATGTC |
| | strand | (SEQ ID NO 10) |
| FUT6 | Upper | CCCACTGTGTACCCTAATGGGTCC |
| | strand | (SEQ ID NO 11) |
| | Lower | CTCTCAGGTGAACCAAGCCGCTAT |
| | strand | (SEQ ID NO 12) |
| FUT7 | Upper | TCGGACATCTTTGTGCCCTATG |
| | strand | (SEQ ID NO 13) |
| | Lower | CGCCAGAATTTCTCCGTAATGTA |
| | strand | (SEQ ID NO 14) |
| β | Upper | ATCTGGCACCACACCTTCTACAATGAGCTGCG |
| actin | strand | (SEQ ID NO 15) |
| | Lower | CGTCATACTCCTGCTTGCTGATCCACATCTGC |
| | strand | (SEQ ID NO 16) |

cDNA was amplified with 25-30 cycles using Pfu Turbo (Stratagene), 0.2 mM of each dNTP, and 100 mM of each primer. The cycle consists of 30 sec at 95° C., 30 sec at 55° C., and 60 sec at 72° C.

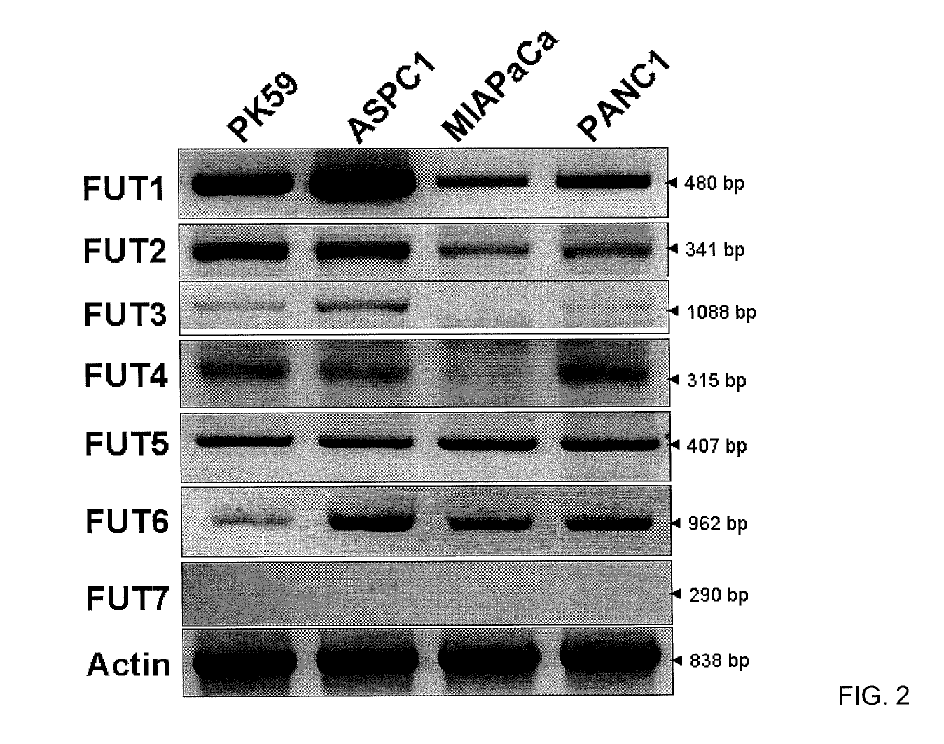

PCR products were subjected to 1.2% agarose-gel electrophoresis, and expression was observed under UV. Results are shown in FIG. 2. Based on the results, PK59 and ASPC-1 were designated to be fucosyltransferase high-expression cell lines, and MIAPaCa and PANC-1 to be fucosyltransferase low-expression cell lines.

### Example 3

### Presence of Fucose Binding Mechanism in Various Pancreatic Cancer Cells

Binding of fucose (fucose refers to L-fucose, unless otherwise stated) to the fucosylated sugar chain high-producing cell line ASPC-1 and fucosylated sugar chain low-producing cell line PANC-1 was investigated using radiolabeled fucose.

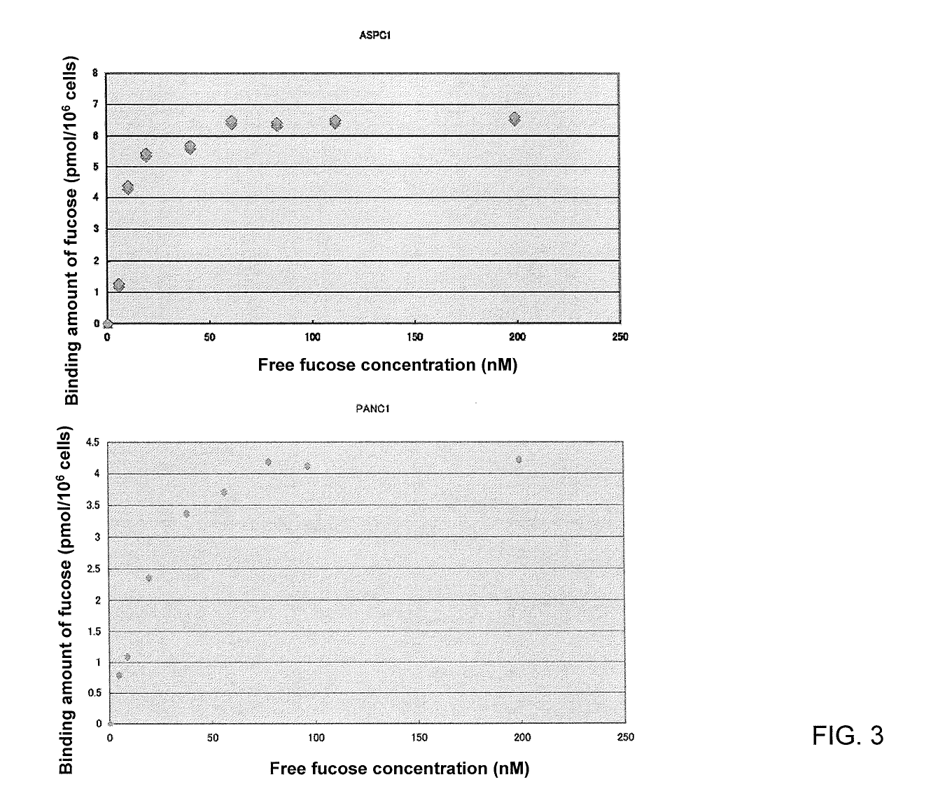

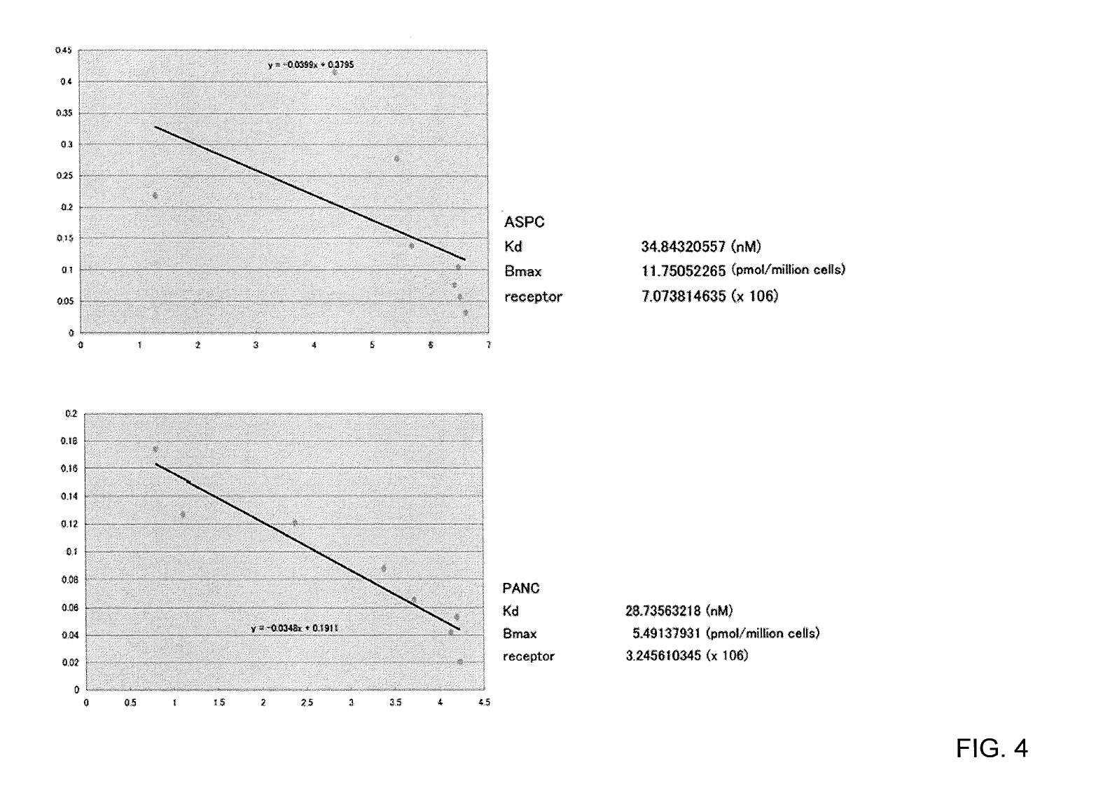

In a 12-well culture plate, 1×10⁵ cells were seeded and cultured overnight, and ¹⁴C-fucose (specific activity: 55 mCi/mmol) diluted with BSA-PBS at a concentration of 0-200 nM was added to the cells and cultured at 4° C. for 1 hr. The cells were washed with cold BSA-PBS, lysed by 1% Triton X100/PBS-0.25% trypsin, and the radioactivity of the ¹⁴C-fucose bound to the cell membrane was measured (FIGS. 3 and 4).

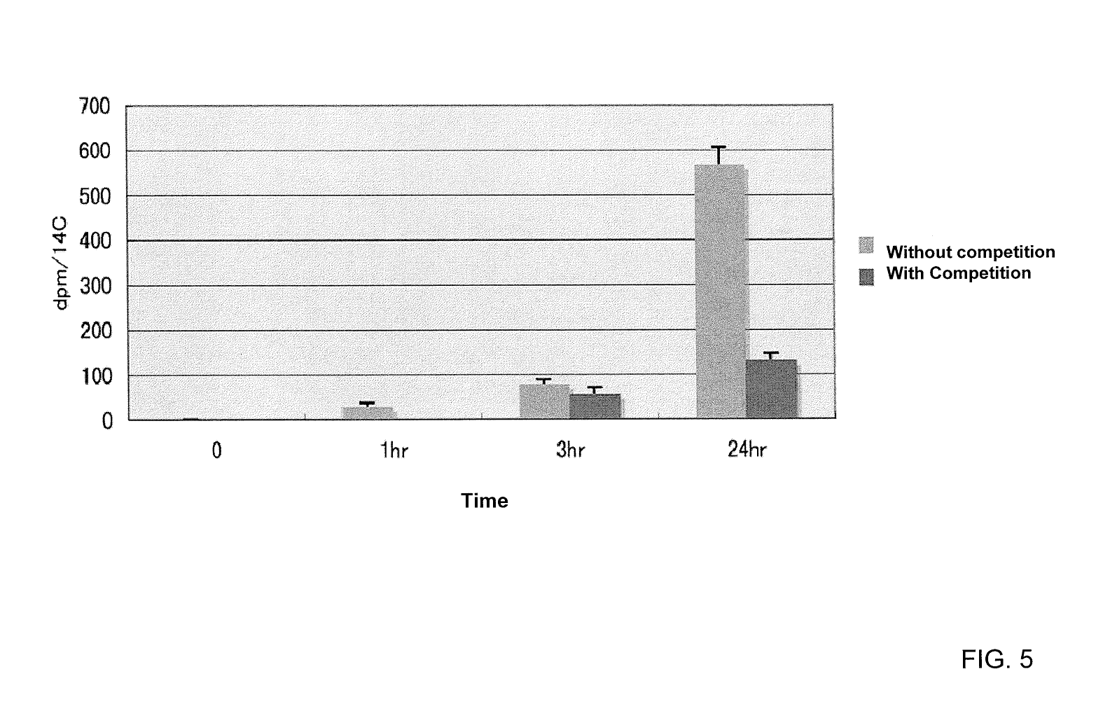

In another experiment, in a 12-well culture plate, 2×10⁵ cells were seeded and cultured overnight, 10 nmol of ¹⁴C-fucose (specific activity: 55 mCi/mmol) were added to the cells either singly or together with 1 pmol of fucose, and cultured at 4° C. for 1, 3, or 24 hr. The cells under each condition were washed with cold BSA-PBS, lysed by 1% Triton X100/PBS-0.25% trypsin, and the radioactivity of the ¹⁴C-fucose was measured (FIG. 5).

From these results, it has been clarified that fucose binds to ASPC-1 and PANC-1 via a receptor-like mechanism, and its affinity is higher in the fucosylated sugar chain high-producing cell line ASPC-1. In addition, because binding of ¹⁴C-fucose was inhibited by non-labeled fucose, this mechanism was clarified to be fucose-specific. This indicates the presence of a fucose-specific receptor-like binding mechanism in fucosylated sugar chain-producing cells, in particular, fucosylated sugar chain high-producing cells.

### Example 4

### Introduction of siRNA by Fucosylated Liposomes

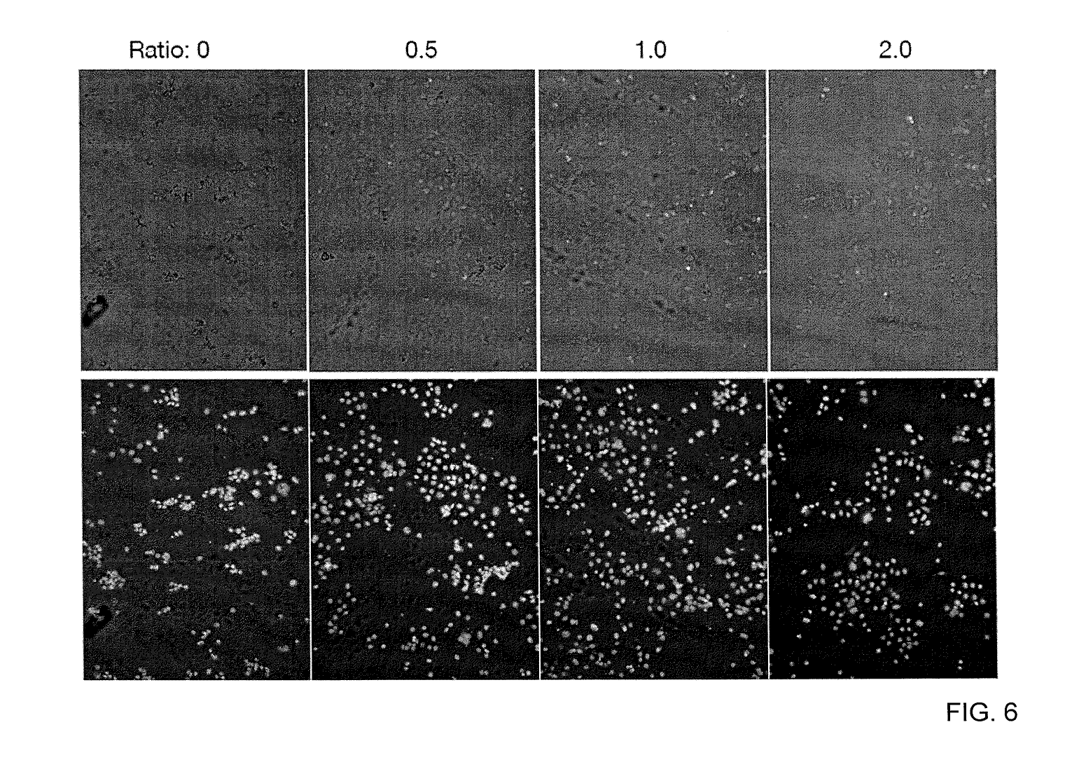

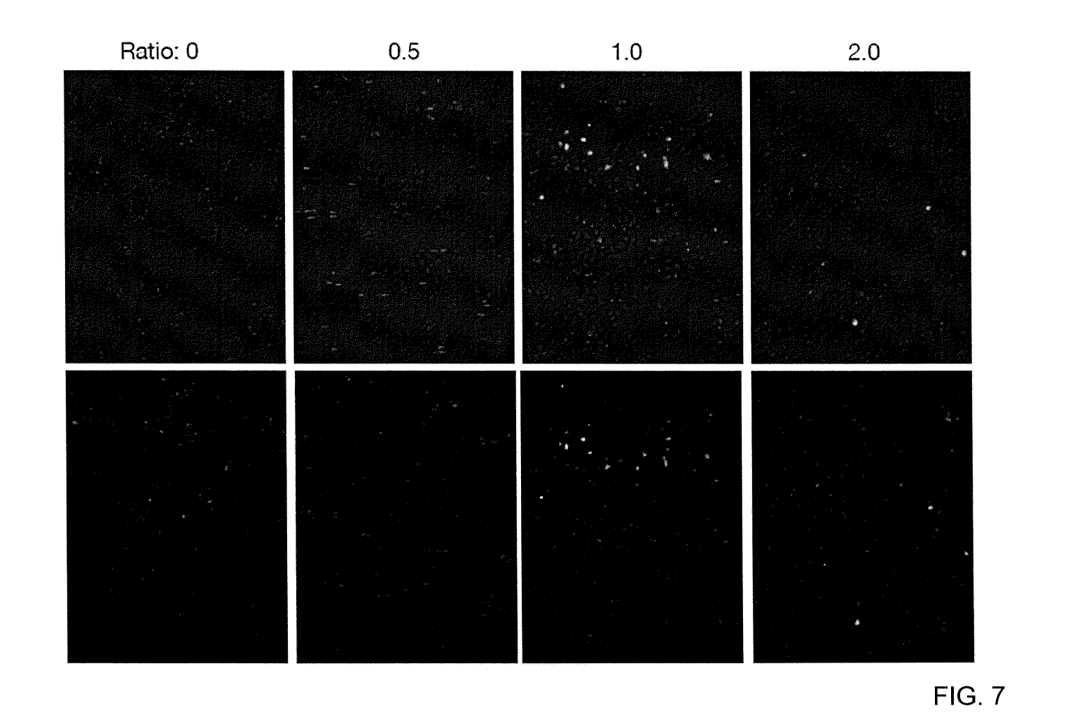

Liposome (Lipotrust, 10 nmol) and L-fucose (0-20 nmol) (SIGMA, MO, USA) were suspended and left at a room temperature for 5 min, then free fucose was removed by a micropartition system (Sartorion VIVASPIN 5000MWCO PES). Next, FAM-labeled siRNA (random) (sense strand: 5′-CGAUUCGCUAGACCGGCUUCAUUGCAG-3′ (SEQ ID NO: 19), antisense strand: 5′-GCAAUGAAGCCGGUCUAGCGAAUCGAU-3′ (SEQ ID NO: 20)) was added and incubated, which was then added to ASPC-1 cells seeded on a chamber slide, and cultured for 1 hr. After culturing, the cells were washed with PBS and fixed with 4% paraformaldehyde, washed with PBS, and counterstained with DAPI and observed under a fluorescence microscope. The result revealed that the molar ratio of liposome:fucose of 1:1 provides the highest introduction efficiency (FIGS. 6 and 7).

### Example 5

### Effect of Fucose on Introduction by Fucosylated Liposomes

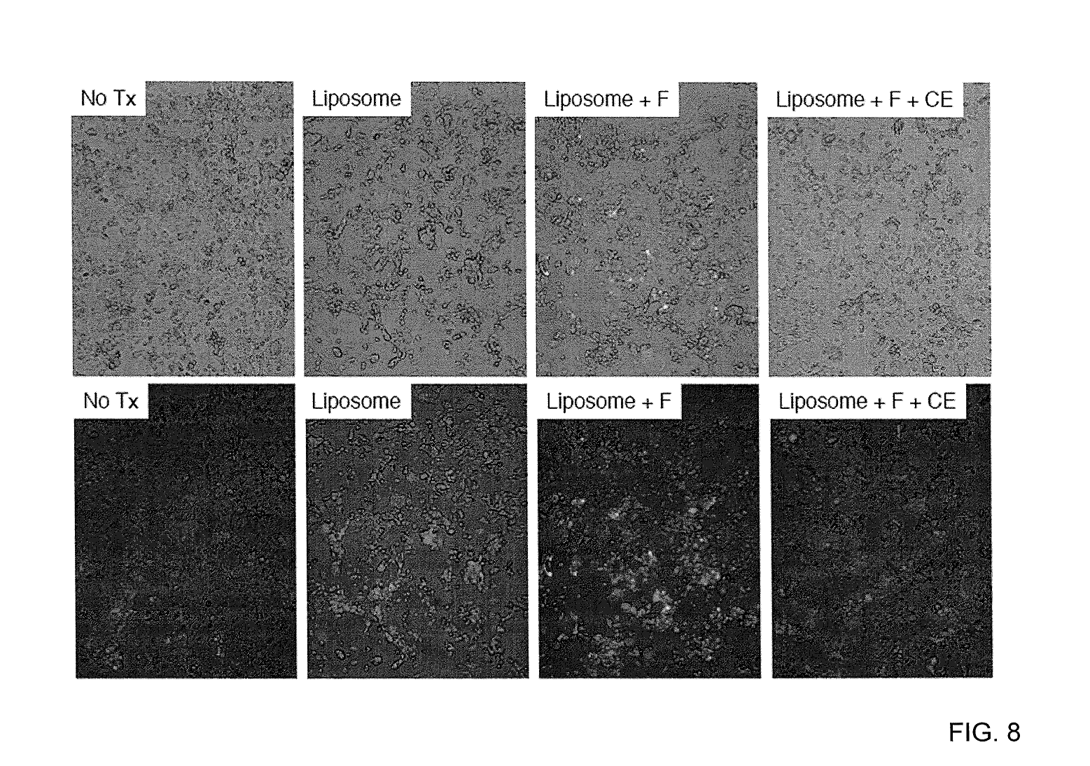

In order to confirm that the introduction of siRNA is due to the fucose-specific receptor-like binding mechanism present in fucosylated molecule-producing cells, introduction efficiency of siRNA under the presence of an excessive amount of fucose was investigated. Liposome (Lipotrust, 10 nmol) and fucose (10 nmol) were suspended, left at a room temperature for 5 min, then free fucose was removed by a micropartition system (Sartorion VIVASPIN 5000MWCO PES). Then, the above FAM-labeled siRNA (random) was added and incubated, which was then added to ASPC-1 cells seeded on a chamber slide, and cultured for 1 hr (Liposome+F). In addition, a group with liposome alone (Liposome), and a group wherein 1 pmol (×100) of fucose was added and pre-incubation was carried out for 10 min before addition of liposomes (Liposome+F+CE), were simultaneously investigated. After culturing, the cells were washed with PBS, fixed with 4% paraformaldehyde, washed with PBS, and counterstained with DAPI and observed under a fluorescence microscope. As a result, introduction of siRNA was significantly suppressed by the presence of fucose (FIG. 8). Namely, introduction of siRNA was inhibited by an excessive amount of fucose, indicating that introduction of siRNA by fucosylated liposomes is via a fucose-specific receptor-like binding mechanism.

### Example 6

### Comparison of siRNA Introduction Efficiency in Various Pancreatic Cancer Cell Lines

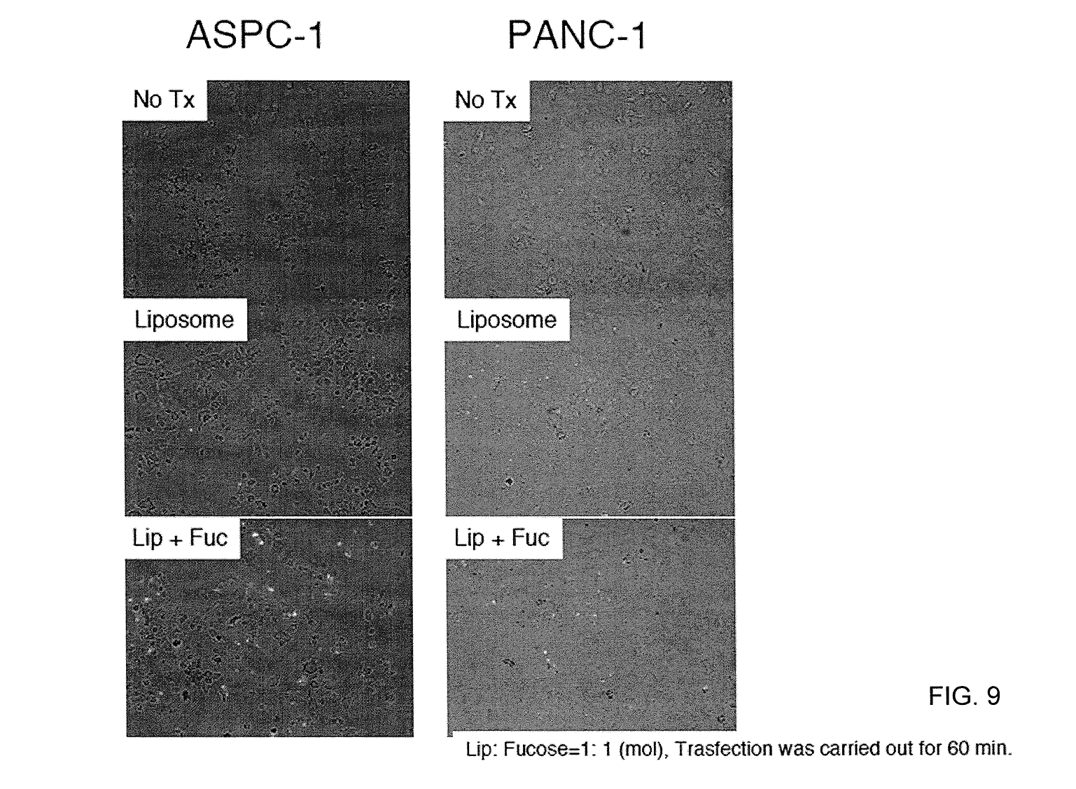

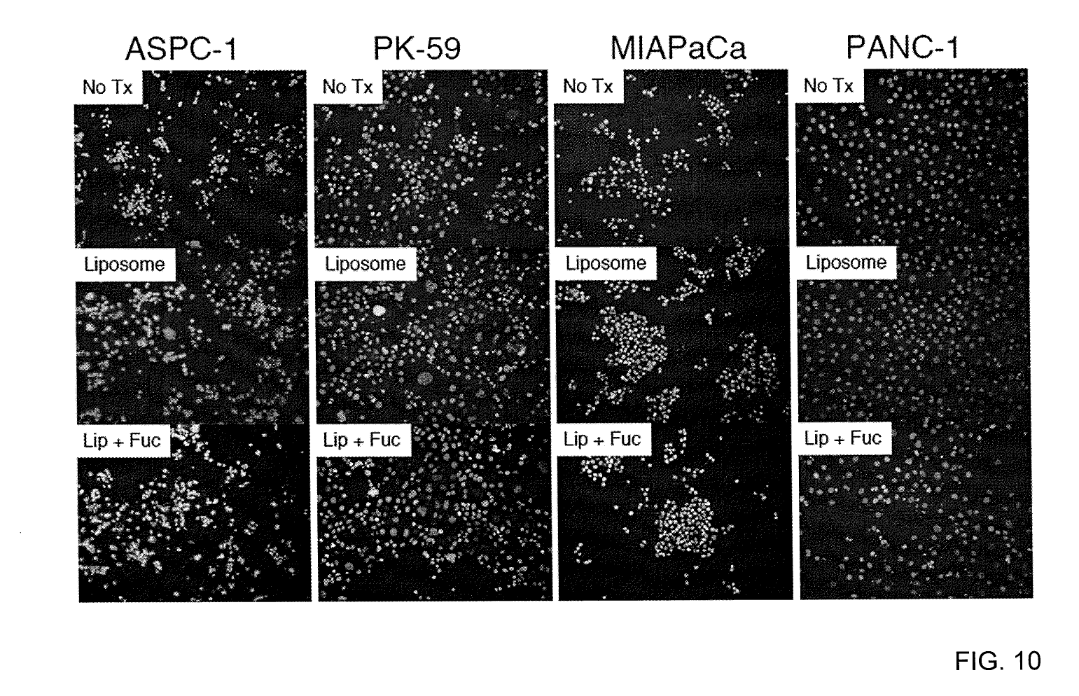

Introduction efficiencies of siRNA by fucosylated liposomes in fucosylated sugar chain high-producing cells and low-producing cells were investigated. In accordance with the above-mentioned procedure, fucosylated liposomes were prepared, and introduction of FAM-siRNA in high-producing cell lines (PK59, ASPC-1) and in low-producing cell lines (MIAPaCa, PANC-1) was observed under a fluorescence microscope. As a result, while a large amount of green FAM was observed inside the cells in the high-producing cell lines, the amount of green FAM inside the cells in the low-producing cell lines was significantly small (FIGS. 9 and 10). This indicates that fucosylated liposomes deliver a substance to cells in a manner depending on the amount of production of fucosylated sugar chains.

### Example 7

### Fucosyltransferase-Dependent Production of CA19-9 in Pancreatic Cancer Cell Line

In order to verify that FUT is a causative gene of CA19-9 production in pancreatic cancer cell lines, cells were transfected with siRNA and expression of various FUT genes was inhibited. siRNA oligonucleotides were prepared in a purified and annealed double-stranded form. Sequences targeting human FUT genes are shown in Table 2.

TABLE 2

| | | | | SEQ |
|---|---|---|---|---|
| | | | | ID |
| Gene | siRNA | * | Nucleic acid sequence (5′->3′) | NO |
| | random- | S | CCUUAUACCUAACGACAGACCCUUU | 21 |
| | FT | AS | AAAGGGUCUGUCGUUAGGUAUAAGG | 22 |
| FUT1 | FT1-1 | S | CCUCCAUAUCCAUCAAGACAGCUUU | 23 |
| | | AS | AAAGCUGUCUUGAUGGAUAUGGAGG | 24 |
| | FT-1-2 | S | CGGACUUGAGAGAUCCUUUCCUGAA | 25 |
| | | AS | UUCAGGAAAGGAUCUCUCAAGUCCG | 26 |
| FUT2 | FT2-1 | S | CACUCUGUCCCGGUUUCCUUCAGCA | 27 |
| | | AS | UGCUGAAGGAAACCGGGACAGAGUG | 28 |
| | FT2-2 | S | CAUCUCUCUUCUGUGAAGAUGCGUU | 29 |
| | | AS | AACGCAUCUUCACAGAAGAGAGAUG | 30 |
| FUT3 | FT3-1 | S | CCGCACUGCUAUUUCAGCUGCUGGU | 31 |
| | | AS | ACCAGCAGCUGAAAUAGCAGUGCGG | 32 |
| | FT3-2 | S | CAGACACGGUCAUCGUGCACCACUG | 33 |
| | | AS | CAGUGGUGCACGAUGACCGUGUCUG | 34 |
| FUT4 | FT4-1 | S | CGAAGCCUGGCAAGUAACCUCUUCA | 35 |
| | | AS | UGAAGAGGUUACUUGCCAGGCUUCG | 36 |
| | FT4-2 | S | GCUACAAGUUCUACCUGGCUUUCGA | 37 |
| | | AS | UCGAAAGCCAGGUAGAACUUGUAGC | 38 |
| FUT5 | FT5-1 | S | UAGGCCAGGGCUUAUGGCAGUGGAA | 39 |
| | | AS | UUCCACUGCCAUAAGCCCUGGCCUA | 40 |
| | FT5-2 | S | CAUCGUGCACCACUGGGAUAUCAUG | 41 |
| | | AS | CAUGAUAUCCCAGUGGUGCACGAUG | 42 |
| FUT6 | FT6-1 | S | GCUGUCUGACCACGCUGCUGUUUCA | 43 |
| | | AS | UGAAACAGCAGCGUGGUCAGACAGC | 44 |
| | FT6-2 | S | ACACGCGGCAUAGCGGCUUGGUUCA | 45 |
| | | AS | UGAACCAAGCCGCUAUGCCGCGUGU | 46 |
| FUT7 | FT7-1 | S | CGCCUCAUCUGCGGGUGGAUGUCUU | 47 |
| | | AS | AAGACAUCCACCCGCAGAUGAGGCG | 48 |
| | FT7-2 | S | GCGGGAACGUUUCUGUGCCAUCUGU | 49 |
| | | AS | ACAGAUGGCACAGAAACGUUCCCGC | 50 |
| FUT8 | FT8-1 | S | CAUCCCAGGUCUGUCGAGUUGCUUA | 51 |
| | | AS | UAAGCAACUCGACAGACCUGGGAUG | 52 |
| | FT8-2 | S | GAGAUAUCAUUGGUGUGGCUGGAAA | 53 |
| | | AS | UUUCCAGCCACACCAAUGAUAUCUC | 54 |

* Sense strand (S), Antisense strand (AS)

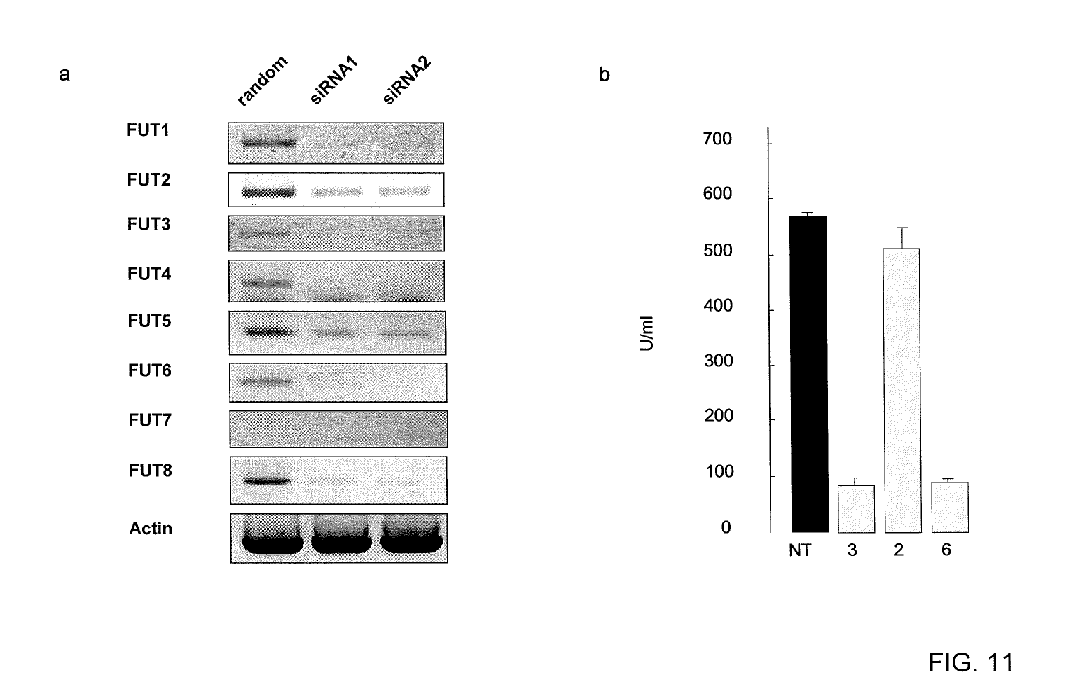

siRNA transfection experiment was performed using 100 nM of siRNA and TransMessenger Transfection Reagent (QIAGEN), in accordance with manufacturer's instructions. 40 hrs after the siRNA transfection, expression of FUT mRNA was analyzed by RT-PCR. Results of inhibition of expression of FUT genes by siRNA were shown in FIG. 11(a).

Of the cells wherein expression of various FUT genes was inhibited, absorption of ¹⁴C-fucose was suppressed in FUT3- or FUT6-knockdown cells. In addition, also in the FUT3- or FUT6-knockdown cells, production of CA19-9 was suppressed (FIG. 11(b)). In the figure, “NT” indicates no treatment, and “2”, “3” and “6” indicate cells wherein siRNA of FUT2, FUT3 and FUT6 was transfected, respectively.

These results suggested that FUT3 and FUT6 are required in the production of CA19-9.

### Example 8

### Preparation of Fucosylated Liposomes Encapsulating (Including) Cy5.5 and CDDP

Fucosylated liposomes encapsulating Cy5.5 and cis-diaminedichloroplatinum(II) (cisplatin, CDDP) of the present invention were prepared as follows. First, CDDP3 was synthesized by a method of Dahara, S., Indian J. Chem. 1970; 7:193-194. Potassium tetrachloroplatinate(II) (4.15 g, 10 mmol) was dissolved in distilled water, potassium iodide (6.64 g, 40 mmol) was added, and stirred on ice under nitrogen atmosphere in the dark for 5 min. Then, aqueous ammonium solution (28%, 1.35 mL) was added to the reaction solution and stirred on ice for 3 hr. Yellow crystal formed was washed with distilled water and ethanol, dried at 40° C. under reduced pressure for 10 hr. In this step, 4.49 g of cis-diaminediiodoplatinum(II) (CDDP2) were obtained. After CDDP2 (2.41 g, 5 mmol) was suspended in distilled water, silver nitride (1.68 g, 9.9 mmol) was added, and stirred on ice in the dark for 24 hr. The reaction solution was filtered through filtering paper to remove silver iodide, and then concentrated using a rotary evaporator, giving white crystal. The crystal was washed with ice-cold distilled water and ethanol, and dried at 40° C. under reduced pressure for 10 hr. The final amount of production of CDDP3 was 1.0 g.

Next, dipalmitoylphosphatidylcholine (DPPC), cholesterol (Chol), ganglioside, dicetyl phosphate (DCP) and dipalmitoylphosphatidylethanolamine (DPPE) were mixed in a molar ratio of 35:40:5:15:5 (456 mg total lipids), and cholic acid (469 mg) was added to facilitate the micelle formation. The mixture was dissolved in a 30-mL methanol/chloroform solution (1:1, v/v). To obtain a lipid thin film, the solvent was evaporated at 37° C. using a rotary evaporator, and dried under reduced pressure. The lipid thin film obtained was dissolved in 30 mL of 10 mM N-[tris(hydroxymethyl)methyl]-3-aminopropanesulfonate (TAPS) buffer (pH 8.4) without NaCl, and subjected to ultrasonic treatment in order to obtain a homogenous micelle suspension.

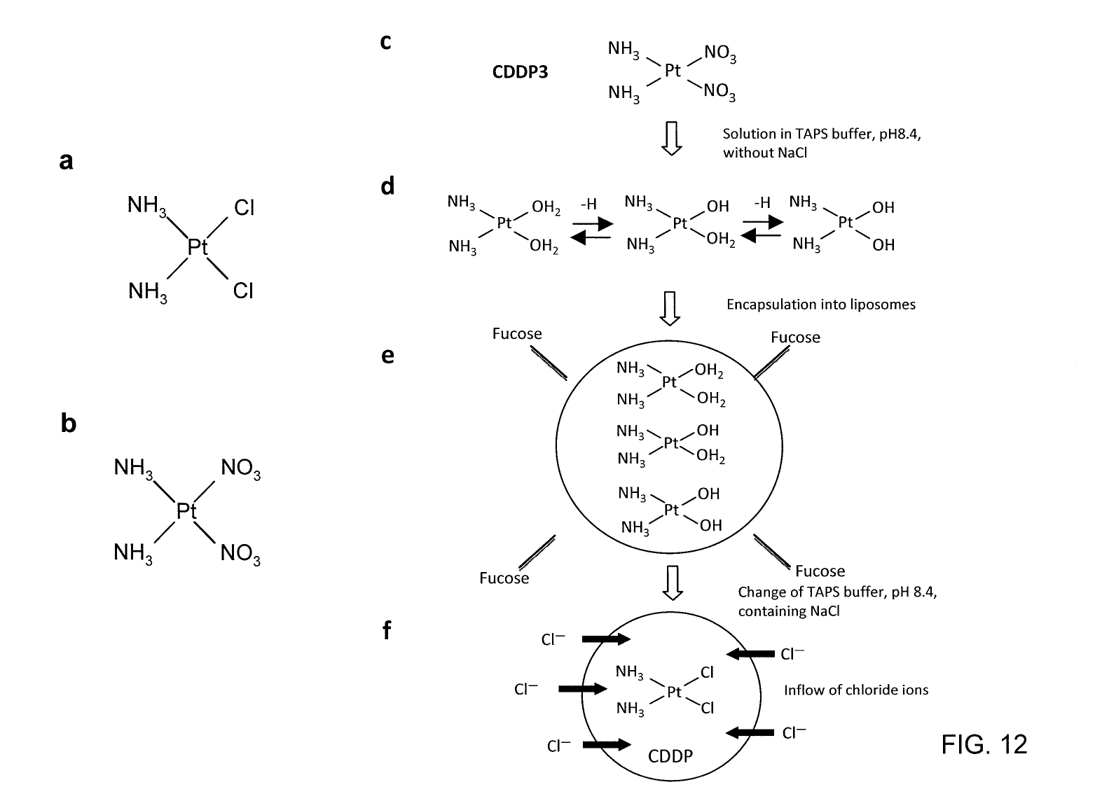

FIG. 12 shows an encapsulation scheme of CDDP. 1 g of CDDP3 was completely dissolved in 70 mL of 10 mM TAPS buffer without NaCl (pH 8.4), and pH is adjusted to 8.4 using 1M NaOH. A solution of CDDP3 and Cy5.5 was added to the above micelle suspension. To remove cholic acid, free CDDP3 and free Cy5.5, the micelle solution was ultrafiltrated with 10 mM TAPS buffer (pH 8.4) using an ultrafiltration disc membrane (molecular weight cut off: 10,000) (Amicon PM10, Millipore) and an ultrafiltration cell holder (Amicon model 8200, Millipore) matched to the membrane. As a result, 100 mL of liposomes encapsulating CDDP3 were obtained. In order to convert CDDP3 in the liposome to CDDP, the obtained liposome was filtered through an ultrafiltration disc membrane (molecular weight cutoff: 300,000) (AmiconXM300, Millipore), and the buffer was changed to 10 mM TAPS buffer comprising 150 mM NaCl (pH 8.4).

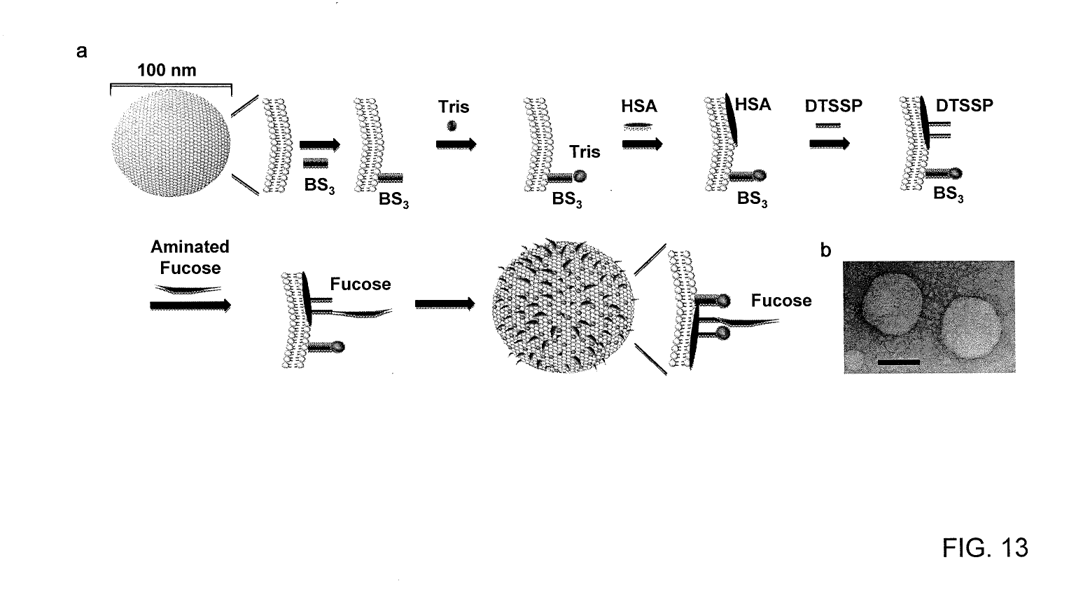

FIG. 13(a) shows a fucosylation scheme of liposomes. In the figure, HSA indicates human serum albumin, BS₃ indicates bis(sulfosuccinimidyl) suberate, “Tris” indicates tris(hydroxymethyl)aminomethane, and DTSSP indicates 3,3′-dithiobis(propionate 3-sulfosuccinimidyl).

A treatment to provide hydrophilic property and binding of L-fucose to the liposome surface were performed following the methods described in Yamazaki, N. J Membrane Sci 1989; 41:249-267 and Yamazaki et al., Methods Enzymol. 1994; 242:56-65. To exchange buffer, the solution was ultrafiltrated with 5 mM sodium hydrogen carbonate buffer (CBS, pH 8.5) through Amicon XM300 membrane. To 100 mL of the liposome solution, 100 mg of a crosslinking agent BS₃ were added, and stirred at 25° C. for 2 hr. After BS₃ bound to the liposome surface, the suspension was stirred at 4° C. overnight. 400 mg of Tris were added and stirred at 25° C. for 2 hr, and further stirred at 4° C. overnight so that Tris bound to BS₃. To remove residual Tris, the suspension was ultrafiltrated with 10 mM TAPS buffer (pH 8.4) through Amicon XM300 membrane.

Furthermore, in accordance with a method described in Hirai et al. 2007, supra, and Hirai et al. 2010, supra, human serum albumin (HSA) was bound to the liposome surface. To acidify the liposome surface, 108 mg of sodium periodate were added to 100 mL of the liposome solution, and stirred at 4° C. overnight. To remove residual sodium periodate, the suspension was ultrafiltrated with 10 mM phosphate buffered saline (PBS, pH 8.0) through Amicon XM300 membrane. Then, 200 mg of HSA were added to the suspension, and stirred at 25° C. for 2 hr. 31.3 mg of sodium cyanoborohydride were added and stirred at 25° C. for 2 hr, then further stirred at 4° C. overnight. To remove residual sodium cyanoborohydride, the solution was ultrafiltrated with CBS buffer (pH 8.5) through Amicon XM300 membrane.

L-fucose was bound to the liposome surface by a crosslinking agent DTSSP. 100 mg of DTSSP were added to 100 mL of the liposome solution, stirred at 25° C. for 2 hr, then further stirred at 4° C. overnight. To remove residual DTSSP, the solution was ultrafiltrated with CBS buffer (pH 8.5) through Amicon XM300 membrane. Fucose reducing terminal was aminated by glycosylamination reaction. 8 mg of fucose were dissolved in 2 mL of distilled water, 1 g of ammonium hydrogen carbonate was added, and stirred at 37° C. for 3 days. Aminated fucose was added such that its final concentration became 10, 25, 50, 100 g/mL, and stirred at 25° C. for 2 hr. Thereafter, in order to repeatedly make the liposome surface hydrophilic, Tris was added such that its final concentration became 132 mg/mL, and stirred at 4° C. overnight. To remove residual fucose and Tris, the solution was ultrafiltrated with HEPES buffer (pH 7.2) through Amicon SM300 membrane.

Non-fucosylated liposomes were prepared similarly to the above-mentioned CDDP-encapsulated fucosylated liposomes, excluding the step of fucose binding. CDDP-encapsulated liposomes and CDDP-encapsulated fucosylated liposomes were ultrafiltrated with 20 mM HEPES buffer (pH 7.2) using Amicon XM300 membrane, and 10-times concentrated.

### Example 9

### Physiochemical Characteristics of Cy5.5-Encapsulated Fucosylated Liposomes

Liposomes were prepared by modified cholic acid dialysis, so that their final concentrations became 25 (F25), 50 (F50), and 100 (F100) μg/mL, as shown in FIG. 13(a), and aminated fucose was crosslinked to these liposomes via DTSSP. Furthermore, to make the liposome surface hydrophilic, BS₃ and Tris were bound. By making the liposome surface hydrophilic, intake of liposomes into the reticuloendothelial system of the liver and spleen, macrophages and vascular endothelial cells can be prevented, and furthermore, adsorption of opsonic proteins in the plasma can be prevented, thereby enabling to keep the liposomes in the blood stream for a longer period of time. From the result of electron microscopic observation shown in FIG. 13(b), almost all fucosylated liposomes were spherical, and the size of the Cy5.5-encapsulated liposomes was approximately 80 nm.

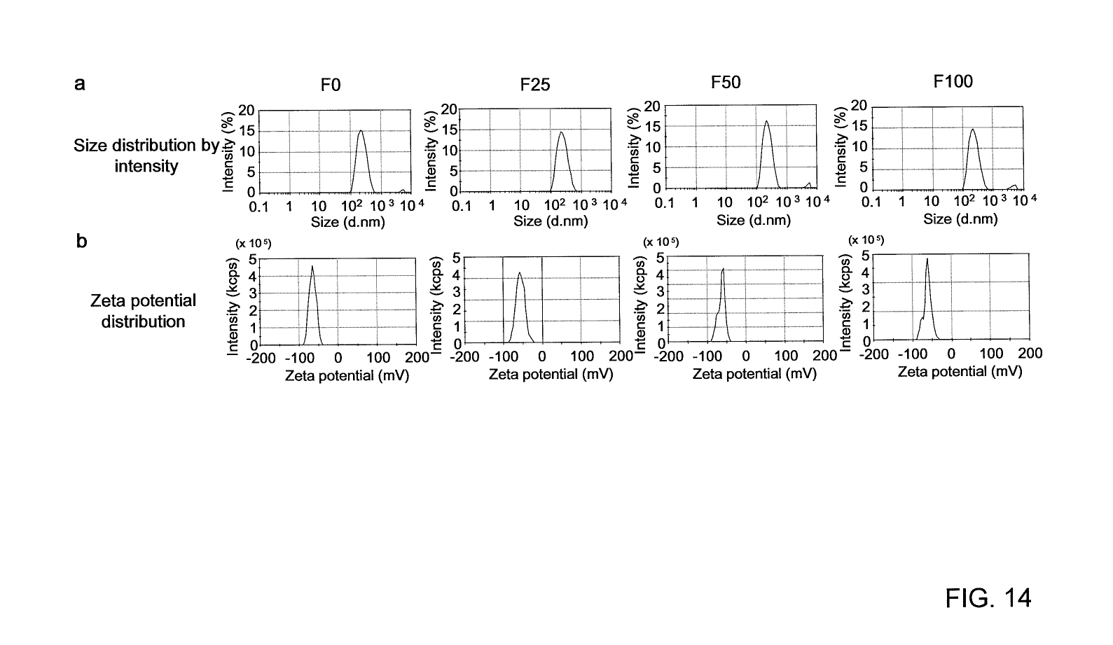

Physiochemical characteristics of Cy5.5-included fucosylated liposomes were shown in FIG. 14 and Table 3.

TABLE 3
(Cy5.5-encapsulated liposomes and Cy5.5-encapsulated
fucosylated liposomes were 10 times concentrated)

| | Fucose concentration in the | | | |
|---|---|---|---|---|
| | binding reaction (μg/mL) | | | |
| | 0 | 25 | 50 | 100 |
| | (F0) | (F25) | (F50) | (F100) |
| Lipid concentration (mg/mL)^{a} | 3.4 | 3.8 | 3.7 | 3.8 |
| Particle size (nm) | 73 | 98 | 72 | 73 |
| Zeta potential (mV)^{b} | −64 | −43 | −45 | −46 |
| Protein concentration (mg/mL)^{c} | 0.7 | 0.7 | 0.8 | 0.7 |
| Protein/lipid weight ratio^{d} | 0.21 | 0.18 | 0.22 | 0.18 |

^{a}Total cholesterol was measured using a cholesterol E test Wako kit.
^{b}Zeta potential was measured using Malvern Nano-S90.
^{c}Protein mass was measured at OD 680 nm.
^{d}Protein/lipid weight ratio was calculated by the equation below:
Protein concentration (mg/mL)/Lipid concentration (mg/mL)

An average particle size and zeta potential of liposomes prepared in water were measured at 25° C. using a dynamic light scattering photometer calibrated with standard latex nanoparticles (Zetasizer Nano-S90, Malvern). The particle sizes measured by Zetasizer Nano-S90 coincided with microscopic observation results, and zeta potential that indicates an electric charge at the surface of liposome membrane was negative at −40 mV or less in each liposome. The particle size distribution after 6-month storage at 4° C. was almost the same as that immediately after preparation, indicating a stable nature of these liposomes.

### Example 10

### Introduction of Cy5.5 Encapsulated in Fucosylated Liposomes

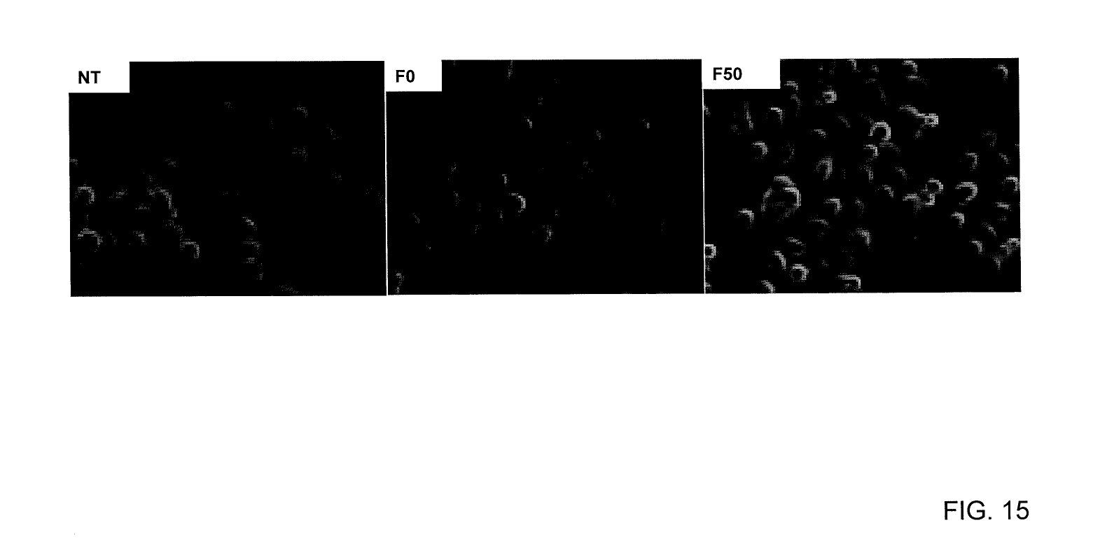

To investigate specific delivery by fucosylated liposomes, a fucosylated liposome that encapsulates Cy5.5 was transfected in CA19-9 producing or CA19-9 non-producing pancreatic adenocarcinoma cells. AsPC-1 cells were incubated with Cy5.5-encapsulated fucosylated liposomes for 2 hr, then washed twice with phosphate buffered saline, and visualized under a fluorescence microscope (FIG. 15). In the AsCP-1 cells which secrete a large amount of CA19-9, fucosylated liposomes (F50), but not fucosylated liposomes (F0) efficiently introduced Cy5.5.

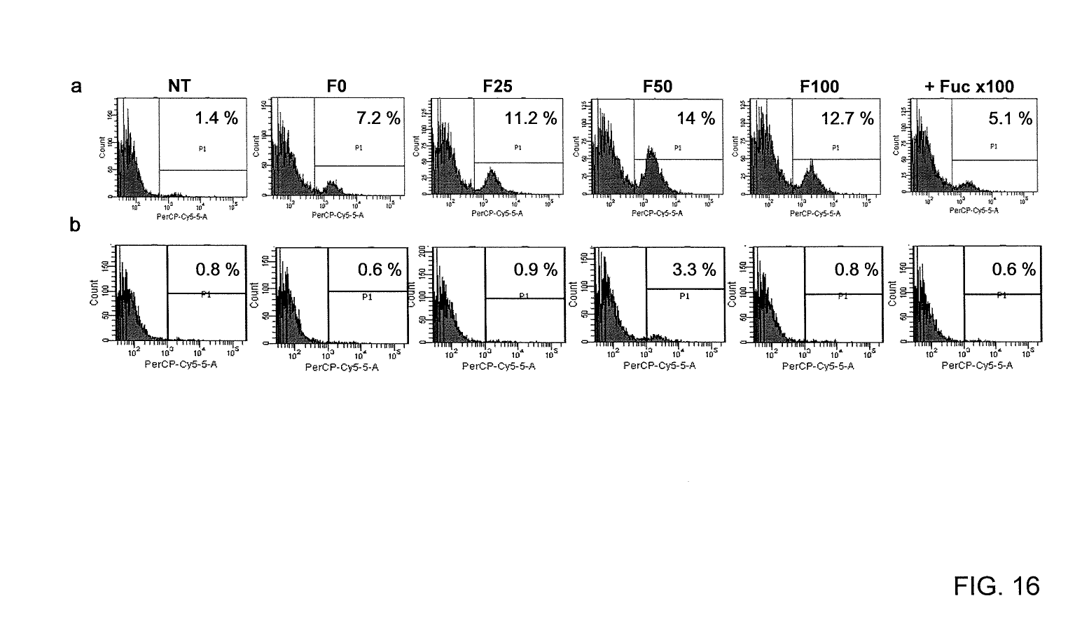

Flow cytometry results (FIG. 16) also showed that fucosylated liposomes (F50) most efficiently transfected Cy5.5 in CA19-9 producing cells (FIG. 16(a)), but not in CA19-9 non-producing cells (FIG. 16(b)). Moreover, because excessive fucose inhibited the efficient introduction (in the figure, +Fuc×100), it is suggested that the introduction of Cy5.5 by fucosylated liposomes is mediated in a fucose receptor-dependent manner.

### Example 11

### Physiochemical Characteristics of CDDP-Encapsulated Fucosylated Liposomes

Table 4 shows physiochemical characteristics of CDDP-encapsulated fucosylated liposomes. The particle size of the CDDP-encapsulated fucosylated liposomes was approximately 200 nm, and the final concentration of CDDP was estimated to be approximately 2 mg/mL.

TABLE 4
Initial amount of CDDP3 (mg) used for the preparation of
1 mL of CDDP-encapsulated liposomes: 100 mg
(CDDP-encapsulated liposomes and CDDP-encapsulated
fucosylated liposomes were 10 times concentrated)

| | Fucose concentration in the | | | |
|---|---|---|---|---|
| | binding reaction (μg/mL) | | | |
| | 0 | 25 | 50 | 100 |
| | (F0) | (F25) | (F50) | (F100) |
| Lipid concentration (mg/mL)^{a} | 8.2 | 8.3 | 8.4 | 8.9 |
| Particle size (nm)^{b} | 232 | 235 | 234 | 229 |
| PDI^{c} | 0.17 | 0.17 | 0.19 | 0.18 |
| Zeta potential (mV)^{d} | −64 | −56 | −63 | −62 |
| CDDP concentration (mg/mL)^{e} | 2.1 | 1.8 | 2.1 | 2.0 |
| CDDP encapsulation efficiency^{f} | 2.1 | 1.8 | 2.1 | 2.0 |
| CDDP/lipid weight ratio^{g} | 0.26 | 0.22 | 0.25 | 0.22 |

^{a}Total cholesterol was measured using a cholesterol E test Wako kit.
^{b}Encapsulation of CDDP3 into liposomes, followed by conversion to CDDP in NaCl-containing TAPS buffer.
^{c}PDI was measured by photon correlation spectrum using Malvern Nano-S90.
^{d}Zeta potential was measured using Malvern Nano-S90.
^{e}An amount of platinum was measured by SHIMADZU AA-6700 atomic absorption spectrometer, and an amount of CDDP was calculated by the equation below:
Amount of platinum × (300/195) wherein “300” indicates the molecular weight of CDDP, and “195” indicates the molecular weight of platinum.
^{f}Encapsulation efficiency was calculated by the equation below:
(Amount of CDDP in liposome/initial amount of chemical substance) × 100
^{g}CDDP/lipid weight ratio was calculated by the equation below:
CDDP concentration (mg/mL)/Lipid concentration (mg/mL)

Analysis of lipid concentration was carried out by the following procedure. CDDP-encapsulated liposomes and CDDP-encapsulated fucosylated liposomes were measured using a cholesterol E-test Wako kit under the presence of 0.5% Triton X-100, in terms of total cholesterol. Lipid concentrations were calculated from the molar ratio of each lipid (4.5) by Eq. (1).

Lipid concentration (mg/mL)=Cholesterol concentration (mg/mL)×4.5 Eq. (1):

Measurement of CDDP, and calculation of CDDP concentration and encapsulation efficiency were carried out as follows. Fucosylated liposomes encapsulating CDDP were 10,000 times diluted with distilled water, and platinum concentration was measured using an automated flameless atomic absorption spectrometer (FAAS) (Model AA-6700, SHIMADZU). Cis-diaminedichloroplatinum was used as a standard substance. A calibration curve of platinum concentration of 50-250 ng/mL was made prior to analysis of each sample. A CDDP amount was calculated by Eq. (2).

CDDP concentration=A×(300/195) Eq. (2):

In Eq. (2), “A” indicates platinum concentration, “300” indicates the molecular weight of CDDP, and “195” indicates the molecular weight of platinum.

Encapsulation efficiency and weight ratio of CDDP to lipid were calculated by Eqs. (3) and (4), respectively.

Encapsulation efficiency (%)=(Amount of CDDP in liposome)/(Initial amount of CDDP)×100 Eq. (3):

Weight ratio of CDDP to lipid=CDDP concentration (mg/mL)/Lipid concentration (mg/mL) Eq. (4):

### Example 12

### Effects of CDDP-Encapsulated Fucosylated Liposomes on Various Pancreatic Cancer Cell Lines

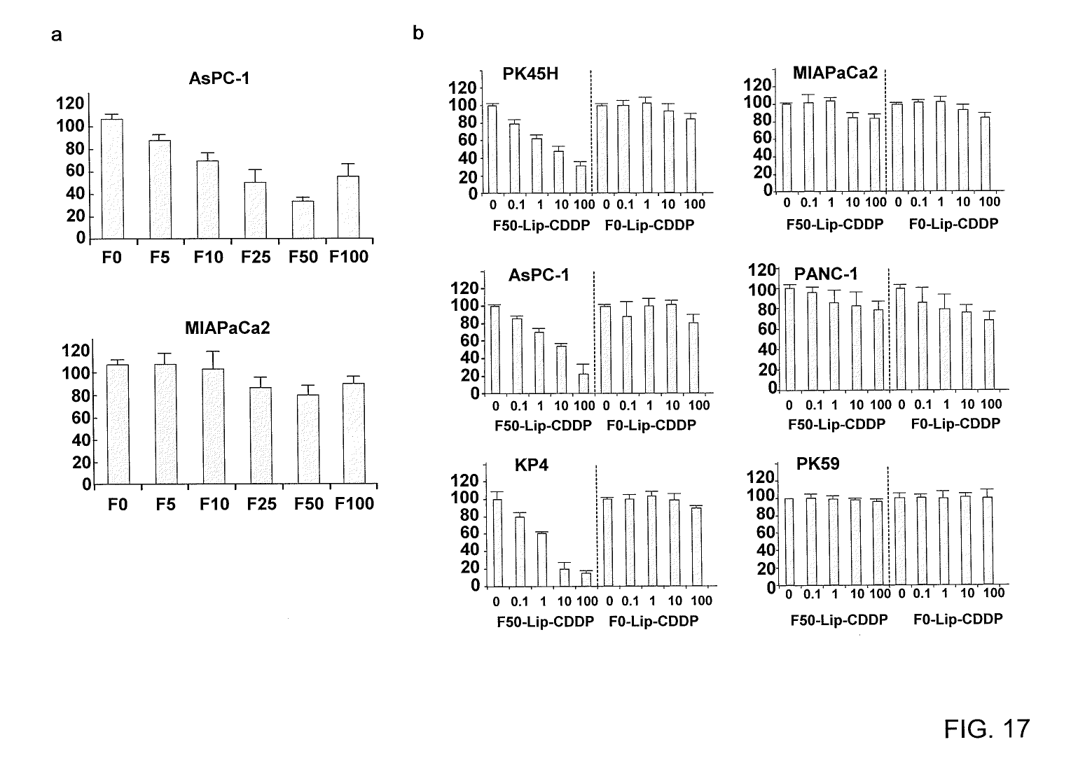

FIG. 17 shows results of investigation of cytotoxic effects of CDDP-encapsulated fucosylated liposomes by WST-1 assay. 2×10⁴ cells from each cell line (AsPC-1, etc.) were transferred to a 24-well plate, and cultured for 1 day with RPMI-1640 supplemented by 10% fetal bovine serum, 5% L-glutamine, and 1% antibiotics. Then, the cells were incubated with various administration amounts of CDDP-encapsulated fucosylated liposomes or CDDP-encapsulated liposomes. After 2 hr incubation, the cells were washed twice with PBS, and finally suspended in RPMI-1640 comprising serum and antibiotics. After 72 hr culturing, WST-1 reagent was added, and growth assay was performed in accordance with the method of Sato, Y. Nat Biotechnol. 2008; 26(4): 431-42. The experiments were triplicated and repeated at least twice.

As a result, CDDP-encapsulated liposomes (F50) (50 μg/mL of fucose bound to liposome) exhibited the highest cytotoxic effect (FIG. 17(a)). In addition, in CA19-9 producing cells (PK45H, AsPC-1 and KP4), CDDP-encapsulated liposomes (F50) are more efficient than CDDP-encapsulated liposomes (F0), indicating a fucose-dependent cytotoxic effect (FIG. 17(b)).

### Example 13

### Investigation of CA19-9 Concentration in Supernatant of Various Colorectal Cancer Cell Cultures

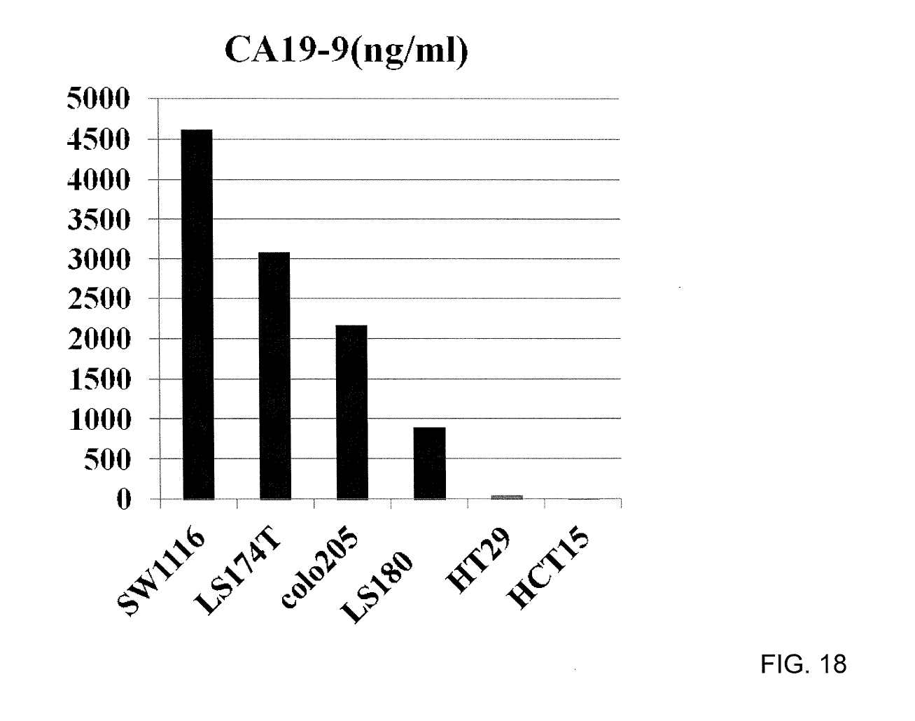

5×10⁶ cells from each colorectal cancer cell line were seeded in a 25-cm² flask, and cultured with 3 ml of a serum-free medium Opti-MEM® for 48 hr. The concentrations of CA19-9 in the supernatant of cultures were investigated by ELISA. Results are shown in FIG. 18 and Table 5.

TABLE 5

| Cell line | CA19-9 concentration (ng/ml) |
|---|---|
| SW1116 | 4620 |
| LS174T | 3080 |
| COLO205 | 2170 |
| LS180 | 890 |
| HT-29 | 39 |
| HCT-15 | <5 |

On the basis of the above results, COLO205 was used as the high-producing cell line and HT-29 was used as the low-producing cell line in the following investigations.

### Example 14

### Introduction of Cy5.5 Encapsulated in Fucosylated Liposomes

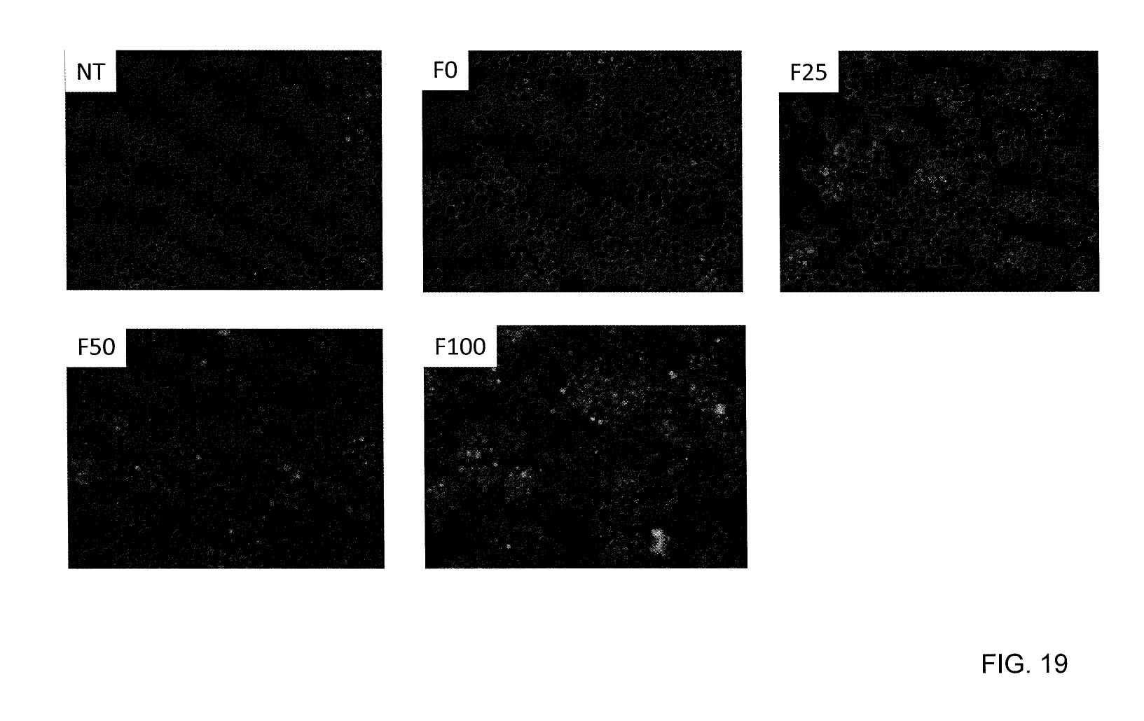

To investigate specific delivery by fucosylated liposomes, first, introduction of Cy5.5 into cells was confirmed by fluorescence microscopy. 1×10⁵ COLO205 cells were seeded on a chamber slide, and incubated with the Cy5.5-included fucosylated liposomes obtained in Example 8 for 2 hr, then washed with phosphate buffered saline (PBS). After fixation with 4% paraformaldehyde, the cells were washed with PBS, counterstained with DAPI, and visualized by a fluorescence microscope (FIG. 19). In the case of liposomes without fucose (F0), almost no red fluorescence of Cy5.5 is observed in the cells; however, it is shown that the fucosylated liposomes (F25, F50 and F100) efficiently introduced Cy5.5.

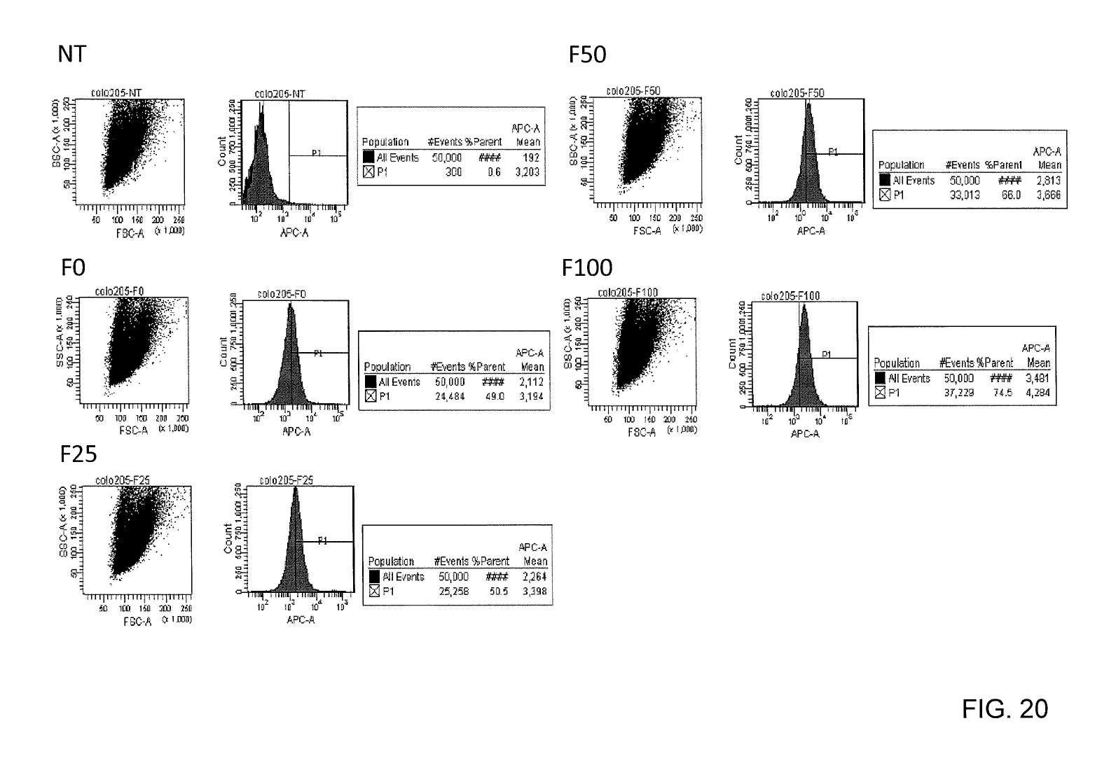

Specific delivery by fucosylated liposomes was also investigated by flow cytometry. 1×10⁶ COLO205 cells were seeded in a 6-well culture flask, and cultured for 2 hr after addition of Cy5.5-included fucosylated liposomes. After culturing, the cells were washed with PBS and the cell suspension was prepared, then Cy5.5-positive cells were detected by FACSCalibur™ flow cytometer (BD Biosciences, San Jose, Calif., USA) (FIG. 20). The results obtained also confirmed efficient introduction of Cy5.5 into cells by fucosylated liposomes.

### Example 15

### Effects of CDDP-Encapsulated Fucosylated Liposomes on Various Colorectal Cancer Cell Lines

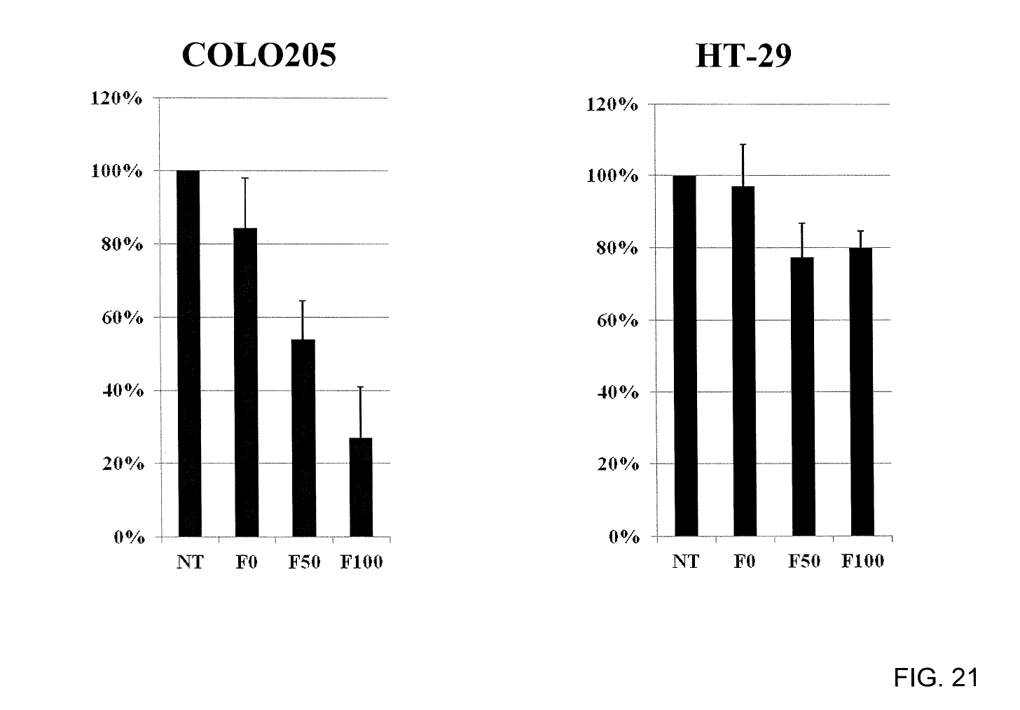

Using WST-1 assay, cytotoxic effects of CDDP-encapsulated fucosylated liposomes were investigated. 2×10⁴ cells of each type were seeded in a 96-well culture flask, and the cells were incubated with liposomes encapsulating CDDP, which are not fucosylated (F0), or which are fucosylated with various degrees (F50, F100). After 2 hr incubation, the cells were washed with PBS, and after replacement of the culture solution (10% FBS-supplemented RPMI1640 medium for COLO205, and 10% FBS-supplemented McCoy's 5A medium for HT-29), further 72 hr of culturing was performed; then an assay using WST-1 reagent was performed similarly to Example 12, and viable cells were counted. From the results shown in FIG. 21, it was confirmed that a higher cytocidal effect by CDDP-encapsulated fucosylated liposomes was observed in the CA19-9 high-producing cell line, compared to the CA19-9 low-producing cell line.

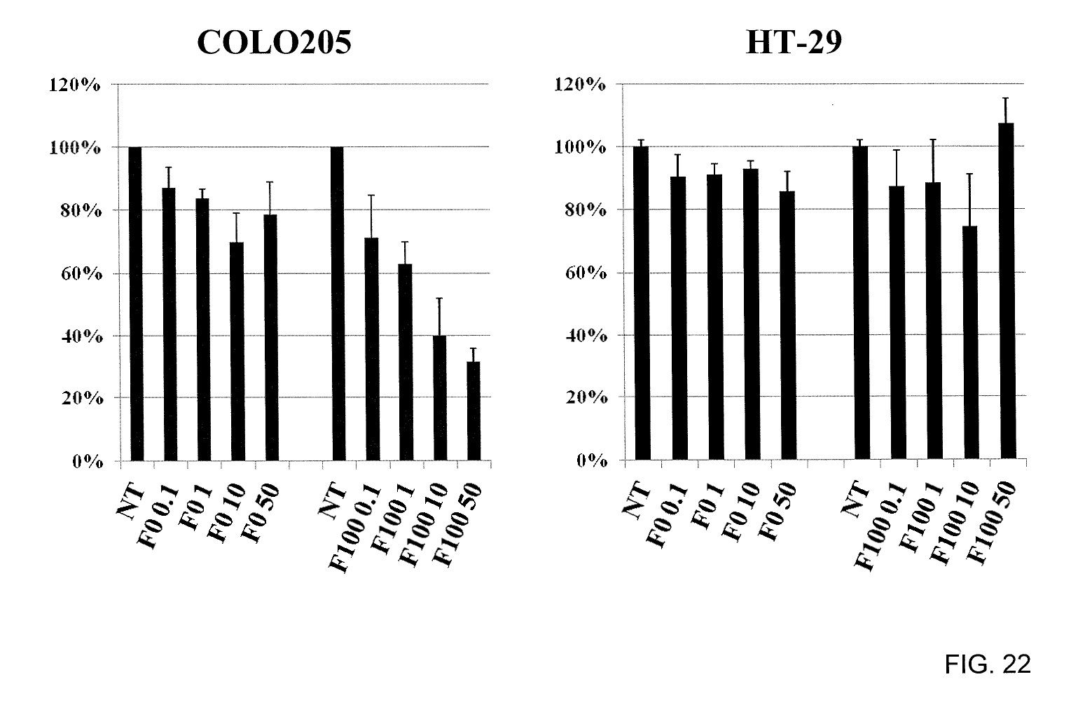

In another experiment, under the same conditions, cells were incubated with fucosylated liposomes including various concentrations of CDDP (F100) or with non-fucosylated liposomes including CDDP (F0), and viable cells were counted similarly (FIG. 22). As a result, it has been clarified that CDDP-included fucosylated liposomes exhibit a cytocidal effect in a dose-dependent manner.

### Example 16

### Investigation of CA19-9 Concentration in Supernatant of Various Biliary Tract Cancer Cell Cultures

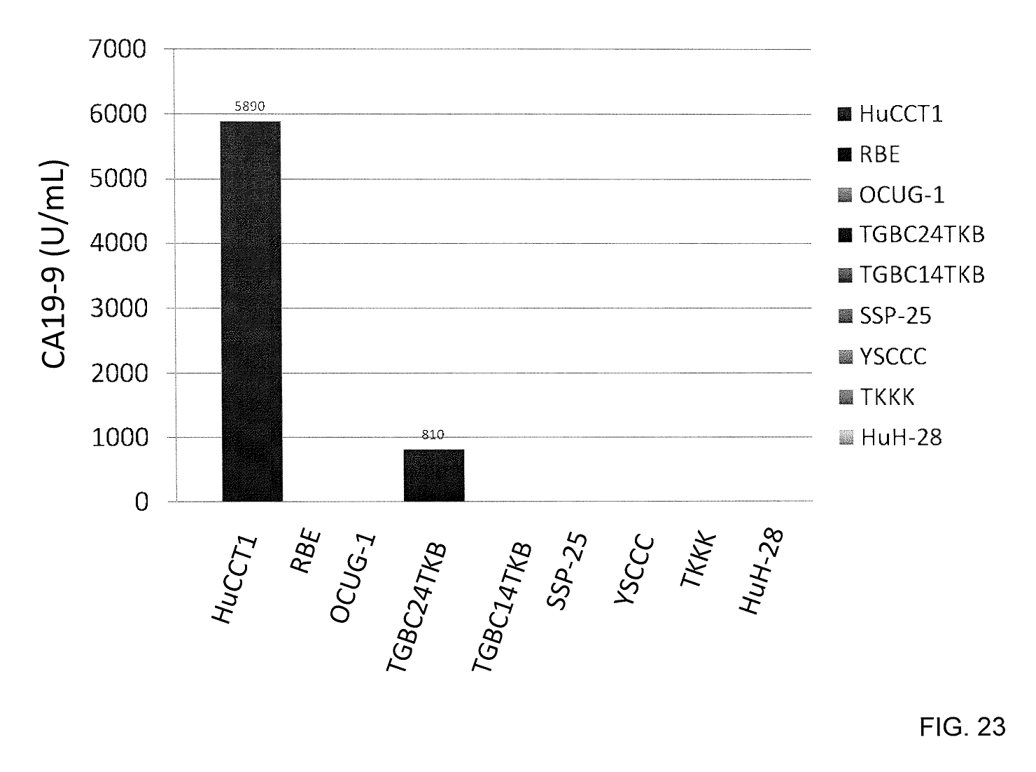

5×10⁶ cells from each biliary tract cancer cell line were seeded in a 25-cm² flask, and cultured with 3 ml of a serum-free medium Opti-MEM® for 48 hr. The concentrations of CA19-9 in the supernatant of cultures were investigated by ELISA. Results are shown in FIG. 23. Based on the results, a high-producing cell line HuCCT1 was used in the following investigation.

### Example 17

### Introduction of Cy5.5 Encapsulated in Fucosylated Liposomes

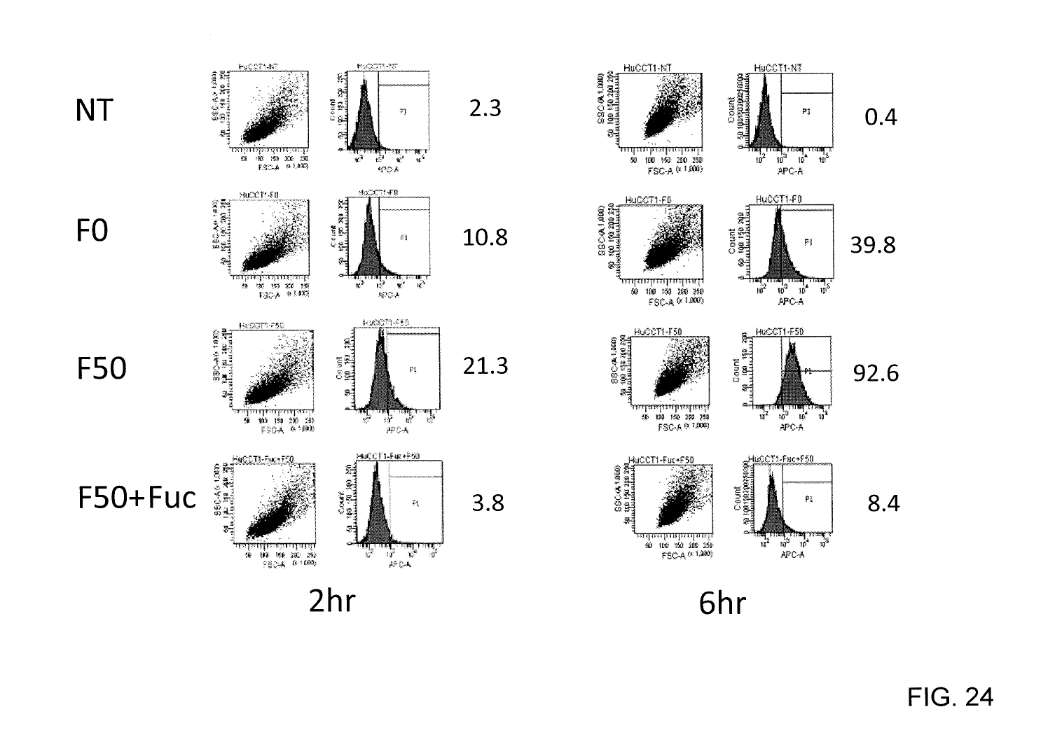

To investigate specific delivery by fucosylated liposomes, introduction of Cy5.5 into cells was confirmed by flow cytometry. 1×10⁶ HuCCT1 cells were seeded in a 6-well culture flask, and the Cy5.5-encapsulated fucosylated liposomes obtained in Example 8 were added and cultured for 2 hr. After culturing, the cells were washed with PBS and the cell suspension was prepared, and Cy5.5-positive cells were detected by FACSCalibur™ flow cytometer (BD Biosciences, San Jose, Calif., USA) (FIG. 24). As a result, it has been clarified that Cy5.5 was introduced more efficiently by fucosylated liposomes (F50) than by non-fucosylated liposomes (F0). In addition, the finding that excessive fucose inhibited the efficient introduction (in the figure, F50+Fuc) indicates that introduction of Cy5.5 by fucosylated liposomes is fucose-receptor dependent.

### Example 18

### Investigation of CA19-9 Concentration in Supernatant of Various Stomach Cancer Cell Cultures

5×10⁶ cells from each stomach cancer cell line were seeded in a 25-cm² flask, and cultured with 3 ml of a serum-free medium Opti-MEM® for 48 hr. The concentrations of CA19-9 in the supernatant of cultures were investigated by ELISA. Results are shown in Table 6.

TABLE 6

| Cell line | CA19-9 concentration (ng/ml) |
|---|---|
| JR-St | 1645 ± 54 |
| HSC-39 | 436 ± 12 |
| NCI-N87 | 204 ± 11 |
| MKN45 | <5 |
| MKN74 | <5 |
| NUGC-4 | <5 |
| KATO-III | 15 ± 4 |

On the basis of the above results, JR-St was used as the high-producing cell line and MKN45 was used as the low-producing cell line in the following investigations.

### Example 19

### Introduction of Cy5.5 Encapsulated in Fucosylated Liposomes

To investigate specific delivery by fucosylated liposomes, introduction of Cy5.5 into cells was confirmed by flow cytometry and fluorescence microscopy.

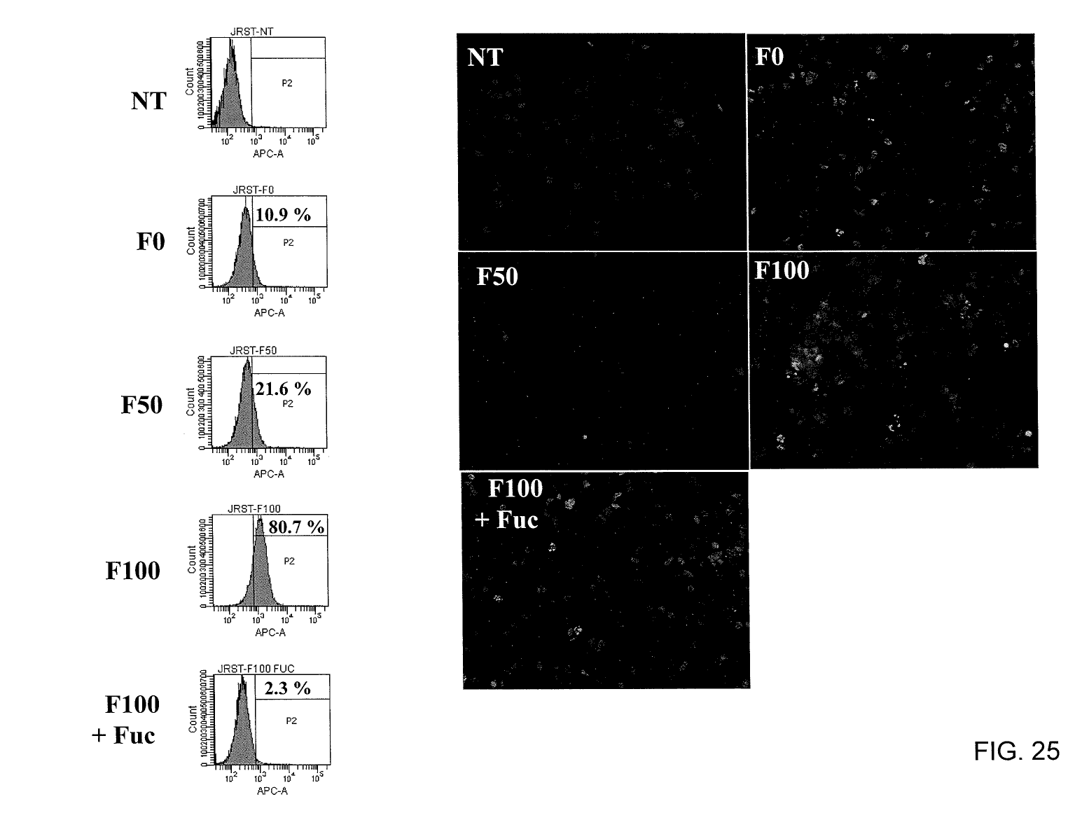

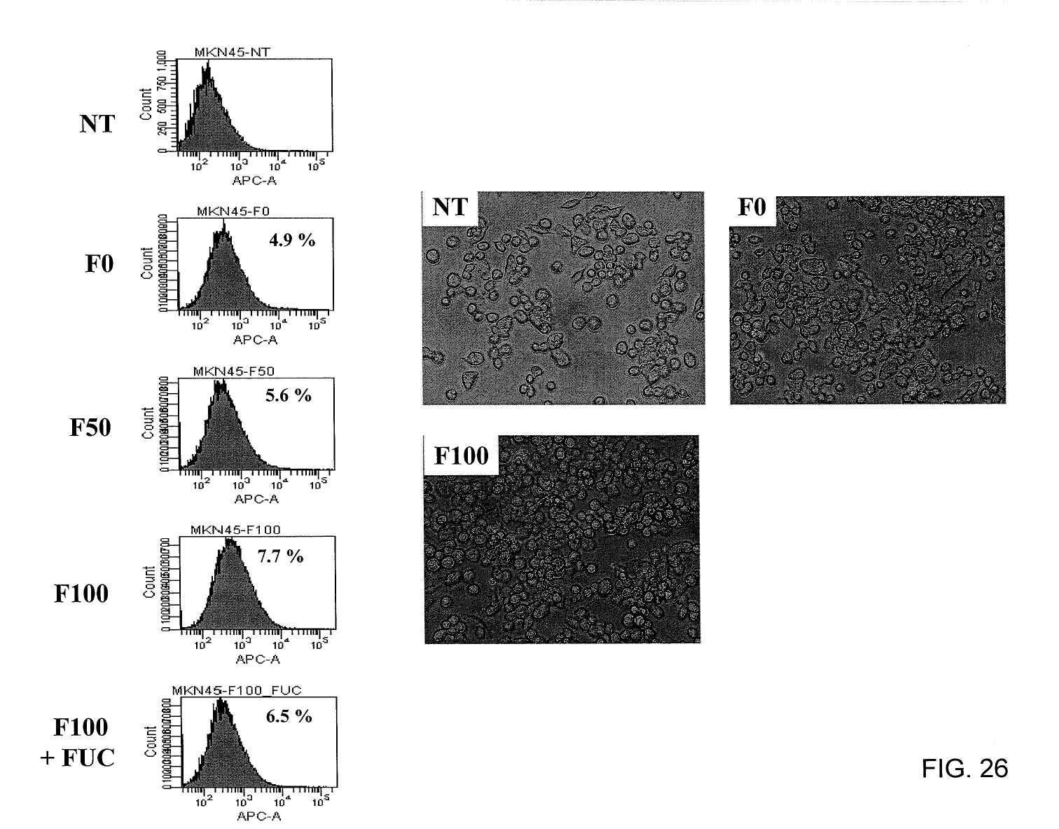

In flow cytometry, 1×10⁶ cells of each type were seeded in a 6-well culture flask, the Cy5.5-included fucosylated liposomes obtained in Example 8 were added, and incubated for 1 hr. After incubation, the cells were washed with PBS and the cell suspension was prepared, then Cy5.5-positive cells were detected by FACSCalibur™ flow cytometer (BD Biosciences, San Jose, Calif., USA). From the results of the charts on the left side of FIGS. 25 and 26, it is shown that, in the high-producing cell line JR-St cells, efficient introduction of Cy5.5 by fucosylated liposomes (F50 and F100) was achieved compared to non-fucosylated liposomes (F0); whereas in the low-producing cell line MKN45 cells, introduction of Cy5.5 even by the fucosylated liposomes (F50 and F100) was at a low level similar to that by non-fucosylated liposomes (F0). In addition, the fact that excessive fucose inhibited the efficient introduction of Cy5.5 by fucosylated liposomes in JR-St cells (in the figure, F100+Fuc) indicates that introduction of Cy5.5 by fucosylated liposomes is fucose-receptor dependent.

In fluorescence microscopy, 1×10⁵ cells of each type were seeded on a chamber slide, and incubated with the Cy5.5-included fucosylated liposomes obtained in Example 8 for 1 hr, then washed with phosphate buffered saline (PBS). After fixation with 4% paraformaldehyde, the cells were washed with PBS, counterstained with DAPI, and visualized by a fluorescence microscope. From the fluorescence microscopic images on the right side of FIGS. 25 and 26, it is shown that, in the high-producing cell line JR-St cells, a larger amount of red fluorescence of Cy5.5 is observed in the cells treated with fucosylated liposomes (F50 and F100) compared to non-fucosylated liposome-treated cells (F0); whereas in the low-producing cell line MKN45 cells, the amount of Cy5.5 introduced into the cells by the fucosylated liposomes (F100) is at a low level similar to that by non-fucosylated liposomes (F0). In addition, the fact that almost no introduction of Cy5.5 into JR-St cells was observed due to the excessive fucose (in the figure, F100+Fuc) indicates that introduction of Cy5.5 by fucosylated liposomes is fucose-receptor dependent, thus supporting the results obtained by flow cytometry.

### Example 20

### Effects of CDDP-Encapsulated Fucosylated Liposomes on Various Stomach Cancer Cell Lines

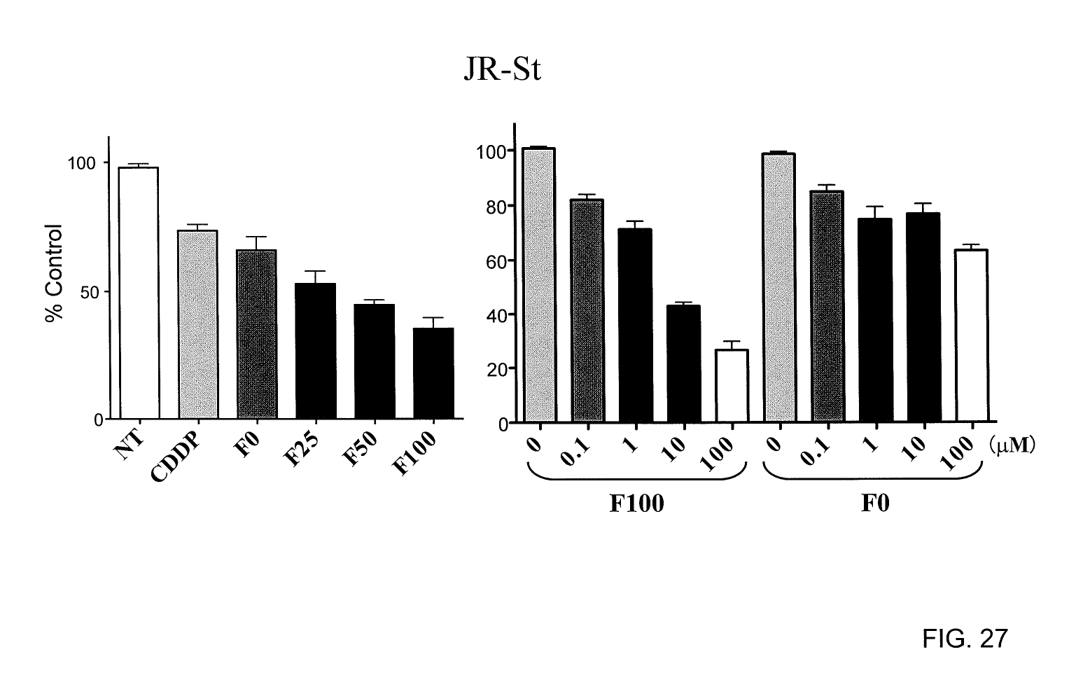

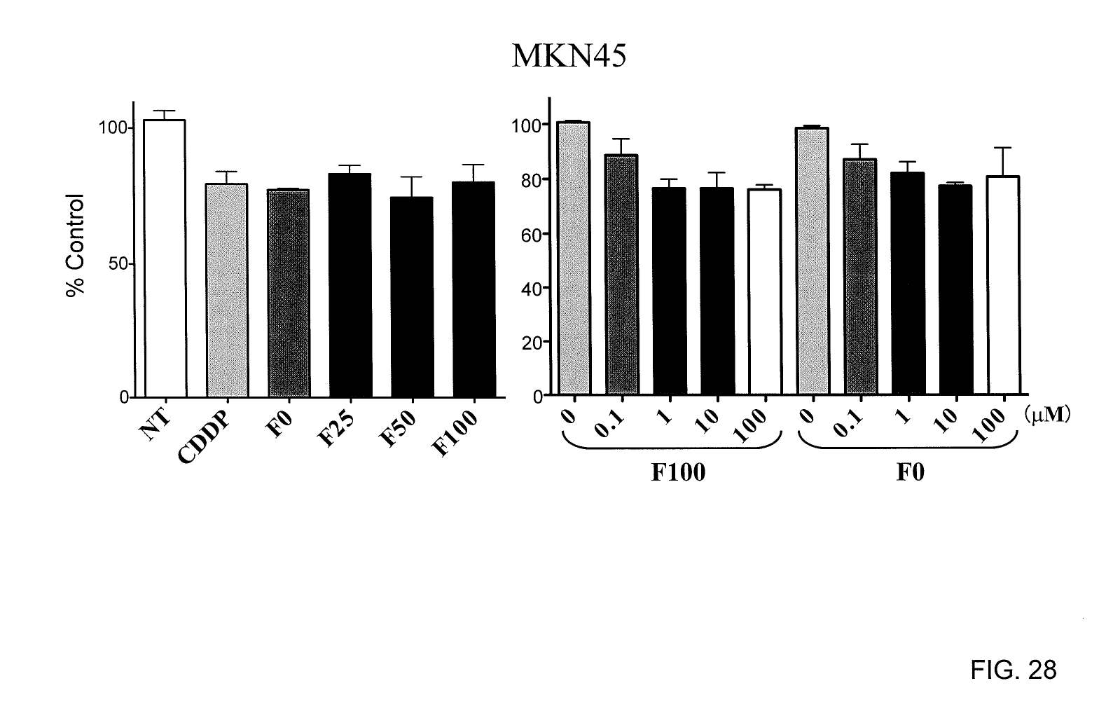

Using WST-1 assay, cytotoxic effects of CDDP-encapsulated fucosylated liposomes were investigated. 2×10⁴ cells of each type were seeded in a 96-well culture flask, and the cells were incubated with liposomes encapsulating CDDP, which were not fucosylated (F0), or which were fucosylated with various degrees (F25, F50, or F100). After 1 hr incubation, the cells were washed with PBS, and after replacement of the culture solution (10% FBS-supplemented RPMI1640 medium), further 72 hr of culturing was performed; then an assay using WST-1 reagent was performed similarly to Example 12, and viable cells were counted. As shown by the results of the graphs on the left side of FIGS. 27 and 28, a higher cytocidal effect by CDDP-included fucosylated liposomes was observed in the CA19-9 high-producing cell line (JR-St), compared to the CA19-9 low-producing cell line (MKN45).

In another experiment, under the same conditions, cells were incubated with various concentrations (0, 0.1, 1, 10 or 100 μM) of CDDP-included fucosylated liposomes (F100) or CDDP-included non-fucosylated liposomes (F0), and viable cells were counted similarly (graphs on the right side of FIGS. 27 and 28). As a result, it has been clarified that CDDP-included fucosylated liposomes exhibit a dose-dependent cytocidal effect in the CA19-9 high-producing cell line.

### Example 21

### Investigation of Expression of CD33 and Notch-1 in Cells of Various Leukemia Cell Lines

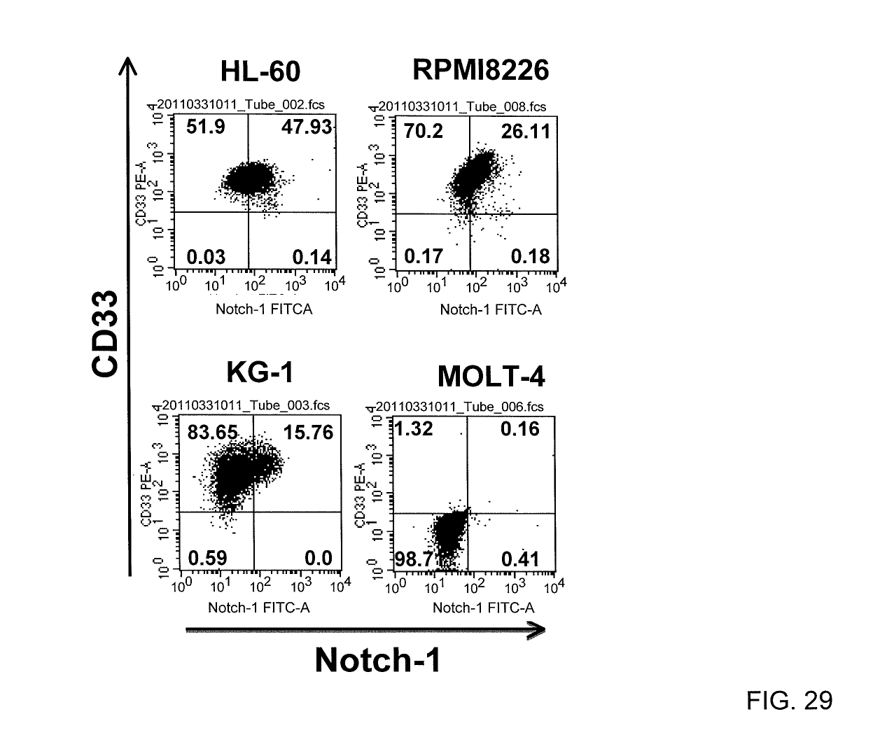

1×10⁶ cells of each leukemia cell line were washed with 0.1% BSA/PBS, and labeled by the reaction for 10 min with 10 μl of antibodies (PE-conjugated CD33 antibody (R&D) and FITC-conjugated Notch-1 antibody (R&D)) in 1 ml of PBS, then the cells were washed with PBS and the cell suspension was prepared. This cell suspension was subjected to FACSCalibur™ flow cytometer (BD Biosiences, San Jose, Calif., USA) to detect positive cells, and they were analyzed by CellQuest Pro software (BD Biosciences). As clearly demonstrated by the results of FIG. 29, both CD33 and Notch-1 were expressed in each of HL-60, KG-1 and RPMI8226 cells, and expression rate of Notch-1 was particularly high in HL-60 cells; however, almost no CD33 and Notch-1 were expressed in MOLT-4.

### Example 22

### Expression of Fucosyltransferase in Various Leukemia Cell Lines

For each leukemia cell line, total RNA was extracted from 1×10⁶ cells and subjected to RT-PCR. Using random hexamer (100 pM) and MMLV (GIBCO), total RNA (1 μg) was reverse-transcribed in accordance with manufacturer's instruction. Primers for each fucosyltransferase are listed in the table below.

TABLE 7
Primers for FUT1-10 and POFUT1

| Name | Type | Sequence |
|---|---|---|
| FUT1 | Upper | ATGTGGCTCCGGAGCCATCGTCAG |
| | strand | (SEQ ID NO 1) |
| | Lower | AGGATCTCTCAAGTCCGCGTACTC |
| | strand | (SEQ ID NO 2) |
| FUT2 | Upper | CTAGCGAAGATTCAAGCCATGTGG |
| | strand | (SEQ ID NO 3) |
| | Lower | GACGTACTCCCCCGGGATGTG (SEQ ID NO 4) |
| | strand | |
| FUT3 | Upper | ATGGATCCCCTGGGTGCAGCCAAG |
| | strand | (SEQ ID NO 5) |
| | Lower | TCAGGTGAACCAAGCCGCTATGCT |
| | strand | (SEQ ID NO 6) |
| FUT4 | Upper | GTGCCCGAAATTGGGCTCCTGCAC |
| | strand | (SEQ ID NO 7) |
| | Lower | GAAGGAGGTGATGTGGACAGCGTA |
| | strand | (SEQ ID NO 8) |
| FUT5 | Upper | CTTATGGCAGTGGAACCTGTCACC |
| | strand | (SEQ ID NO 9) |
| | Lower | CCAGCCGTAGGGCGTGAAGATGTC |
| | strand | (SEQ ID NO 10) |
| FUT6 | Upper | CCCACTGTGTACCCTAATGGGTCC |
| | strand | (SEQ ID NO 11) |
| | Lower | CTCTCAGGTGAACCAAGCCGCTAT |
| | strand | (SEQ ID NO 12) |
| FUT7 | Upper | TCGGACATCTTTGTGCCCTATG (SEQ ID NO 13) |
| | strand | |
| | Lower | CGCCAGAATTTCTCCGTAATGTA |
| | strand | (SEQ ID NO 14) |
| FUT8 | Upper | TGCCTGGGGGACCTTGCTGT (SEQ ID NO 15) |
| | strand | |
| | Lower | CCCGCCAATCCTGCTCCA (SEQ ID NO 16) |
| | strand | |
| FUT9 | Upper | CTTACCGCCGTGATTCAGAT (SEQ ID NO 55) |
| | strand | |
| | Lower | AATGCTTGCCCGTAGGTATG (SEQ ID NO 56) |
| | strand | |
| FUT10 | Upper | TCGGACATCTTTGTGCCCTATG (SEQ ID NO 57) |
| | strand | |
| | Lower | TTTCAGTGGCCTCCAGAACT (SEQ ID NO 58) |
| | strand | |
| POFUT1 | Upper | GAAGGAAGGAAACCCCTTTG (SEQ ID NO 59) |
| | strand | |
| | Lower | TCTCCCGTCTTCACCATTTC (SEQ ID NO 60) |
| | strand | |
| β- | Upper | ATCTGGCACCACACCTTCTACAATGAGCTGCG |
| actin | strand | (SEQ ID NO 17) |
| | Lower | CGTCATACTCCTGCTTGCTGATCCACATCTGC |
| | strand | (SEQ ID NO 18) |

cDNA was amplified with 25-30 cycles using Pfu Turbo (Stratagene), 0.2 mM of each dNTP, and 100 pM of each primer. Each cycle consists of 30 sec at 95° C., 30 sec at 55° C., and 60 sec at 72° C.

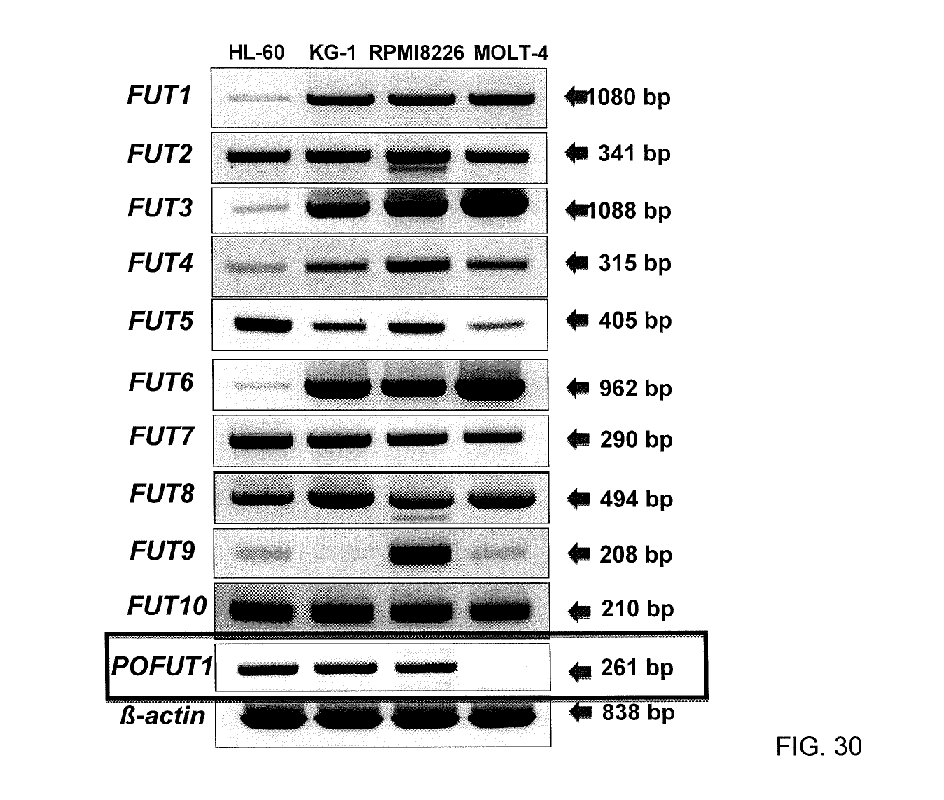

PCR products were subjected to 1.2% agarose-gel electrophoresis, and expression was observed under UV. From the results shown in FIG. 30, it is clarified that POFUT1, which is considered to be involved in the fucosylation of Notch-1, is expressed in each of Notch-1-positive HL-60, KG-1 and RPMI8226 cells, whereas almost no POFUT1 is expressed in Notch-1-negative MOLT-4. Based on this finding, HL-60 was used as the Notch-1 expressing strain, and MOLT-4 was used as the Notch-1 non-expressing strain in the following investigations.

### Example 23

### Introduction of Fluorescent Labels Encapsulated in Fucosylated Liposomes

To investigate specific delivery by fucosylated liposomes, introduction of fluorescent labels into cells was confirmed by flow cytometry and fluorescence microscopy.

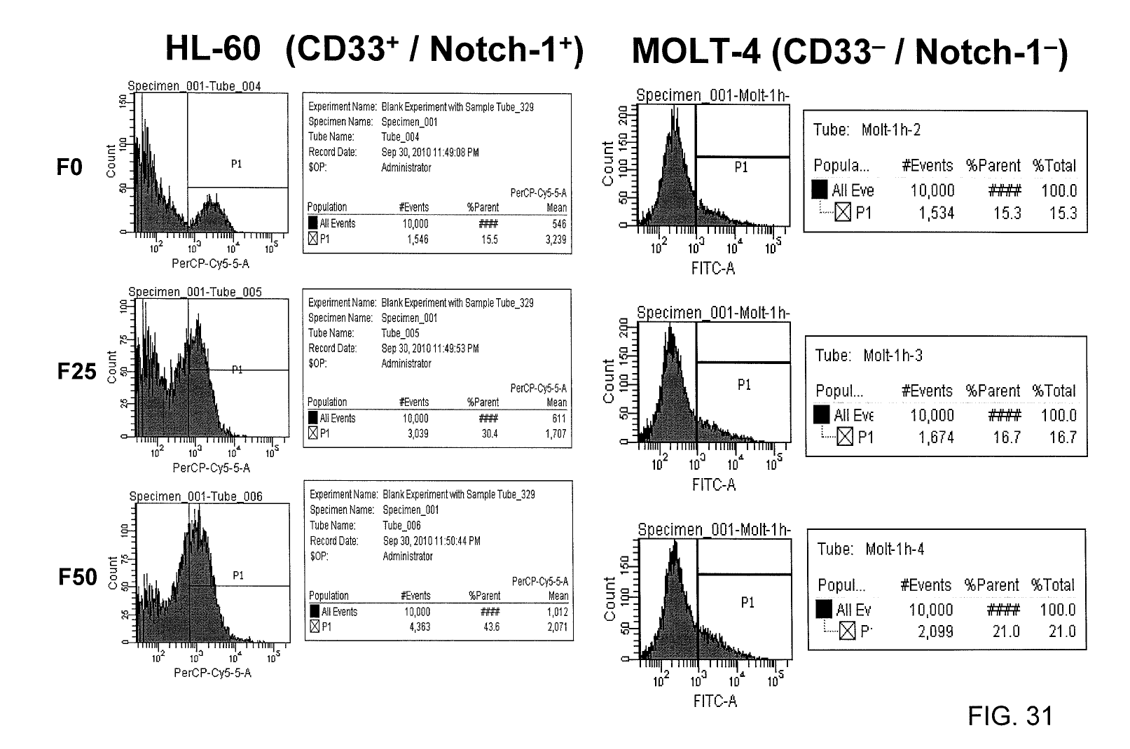

Inflow cytometry, 1×10⁶ cells of each type were seeded in a 6-well culture flask, Cy5.5-included fucosylated liposomes (HL-60 cells) obtained in Example 8 or FAM-included fucosylated liposomes (MOLT-4 cells) obtained similarly to Example 8 were added, and they were incubated for 2 hr. After incubation, the cells were washed with PBS, and the cell suspension was prepared, then fluorescent-labeled positive cells were detected by FACSCalibur™ flow cytometer (BD Biosciences, San Jose, Calif., USA). From the results shown in FIG. 31, it is clarified that in Notch-1 expressing HL-60 cells, fluorescent labels are efficiently introduced by fucosylated liposomes (F25 and F50) compared to non-fucosylated liposomes (F0); whereas in Notch-1 non-expressing MOLT-4 cells, introduction of fluorescent labels even by the fucosylated liposomes is at a low level similar to the case of non-fucosylated liposomes.

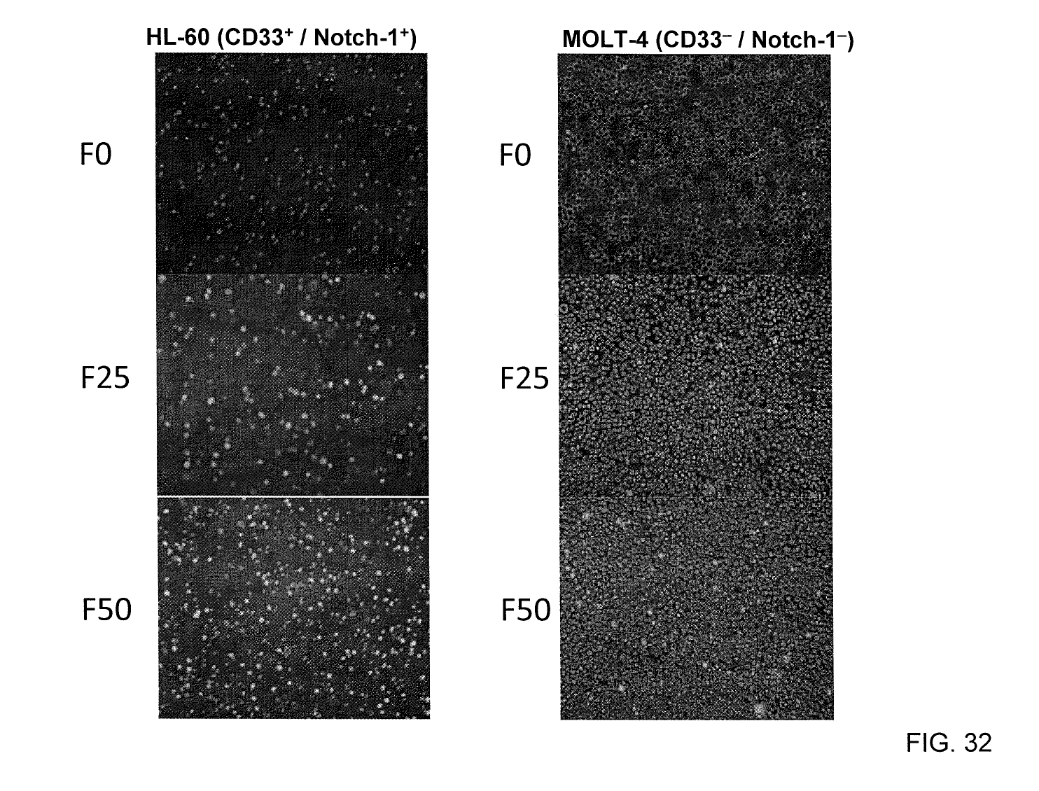

In fluorescence microscopy, 1×10⁵ cells of each type were seeded on a chamber slide, and incubated with the FAM-included fucosylated liposomes obtained similarly to Example 8 for 2 hr, then washed with phosphate buffered saline (PBS). After fixation with 4% paraformaldehyde, the cells were washed with PBS, counterstained with DAPI, and visualized by a fluorescence microscope. In the fluorescence microscopic images in FIG. 32, in Notch-1 expressing cell line HL-60 cells, a significantly larger amount of fluorescence of FAM was observed in the fucosylated liposome-treated cells (F25 and F50), compared to non-fucosylated liposome-treated cells (F0); whereas in Notch-1 non-expressing cell line MOLT-4 cells, the amount of FAM introduced in the cells even by the fucosylated liposomes was at a low level similar to that by the non-fucosylated liposomes, thus supporting the results of flow cytometry.

### Example 24

### Effects of Doxorubicin-Encapsulated Fucosylated Liposomes on Various Leukemia Cell Lines

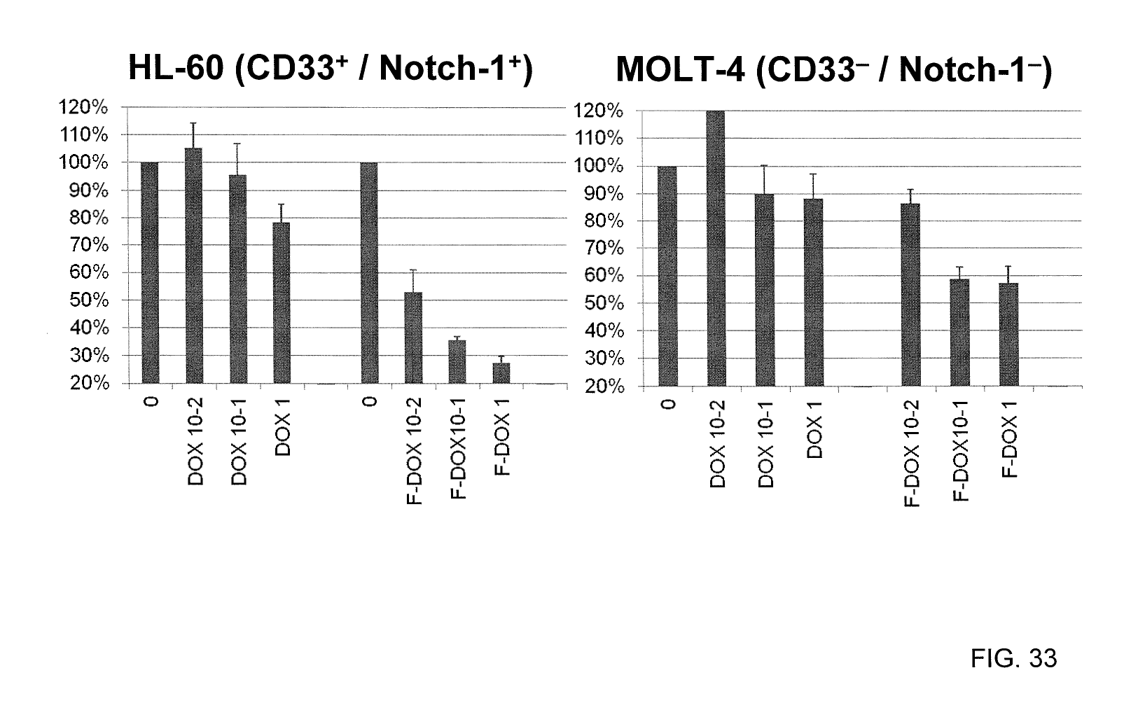

Using WST-1 assay, cytotoxic effects of doxorubicin-encapsulated fucosylated liposomes were investigated. Doxorubicin-included fucosylated liposomes were prepared by a method similar to Example 8. 2×10⁴ cells of each type were seeded in a 96-well culture flask, and the cells were incubated with various concentrations of doxorubicin-encapsulated fucosylated liposomes (F-DOX) or with doxorubicin alone (DOX). After 2 hr incubation, the cells were washed with PBS, and after replacement of the culture solution (10% FBS-supplemented RPMI1640 medium), further 72 hr of culturing was performed; then an assay using a WST-1 reagent was performed similarly to Example 12, and viable cells were counted. As shown in FIG. 33, in the Notch-1 expressing cell line (HL-60), doxorubicin-included fucosylated liposomes (F-DOX) exhibited a significant dose-dependent cytocidal effect compared to doxorubicin alone (DOX). In contrast, in the Notch-1 non-expressing cell line (MOLT-4), such a dose-dependent cytocidal effect was not observed.

### Example 25

### Investigation of Expression of CD33 and Notch-1 in Samples from Leukemia Patients

Regarding samples from leukemia patients, peripheral blood was collected after obtaining patient consent, then mononuclear cells were separated by Ficoll-Hypaque and stored in liquid nitrogen before use. Conditions of patients from which samples were collected are listed in Table 8 below.

TABLE 8

| case No. | Diagnosis | sex | Age | Tx | |
|---|---|---|---|---|---|
| 1 | AML M1 | M | 81 | BSC | dead |
| 2 | AML M2 | F | 57 | IDR/AraC | alive |
| 3 | AML M2 | M | 66 | IDR/AraC | alive |
| 4 | ALL | F | 71 | ALL202 | alive |
| 5 | AML M2 | M | 64 | IDR/AraC | dead |
| 6 | AML M2 | M | 44 | BMT | alive |
| 7 | AML M3 | M | 49 | dead | |
| 8 | AML M2 | M | 66 | IDR/AraC | alive |
| 9 | AML M4 | F | 43 | IDR/AraC | dead |
| 10 | AML M2 | F | 21 | BMT | dead |
| 11 | AML M4 | M | 64 | BMT | alive |
| 12 | AML M6 | M | 64 | BMT | alive |
| 13 | AML M2 | F | 80 | BSC | dead |

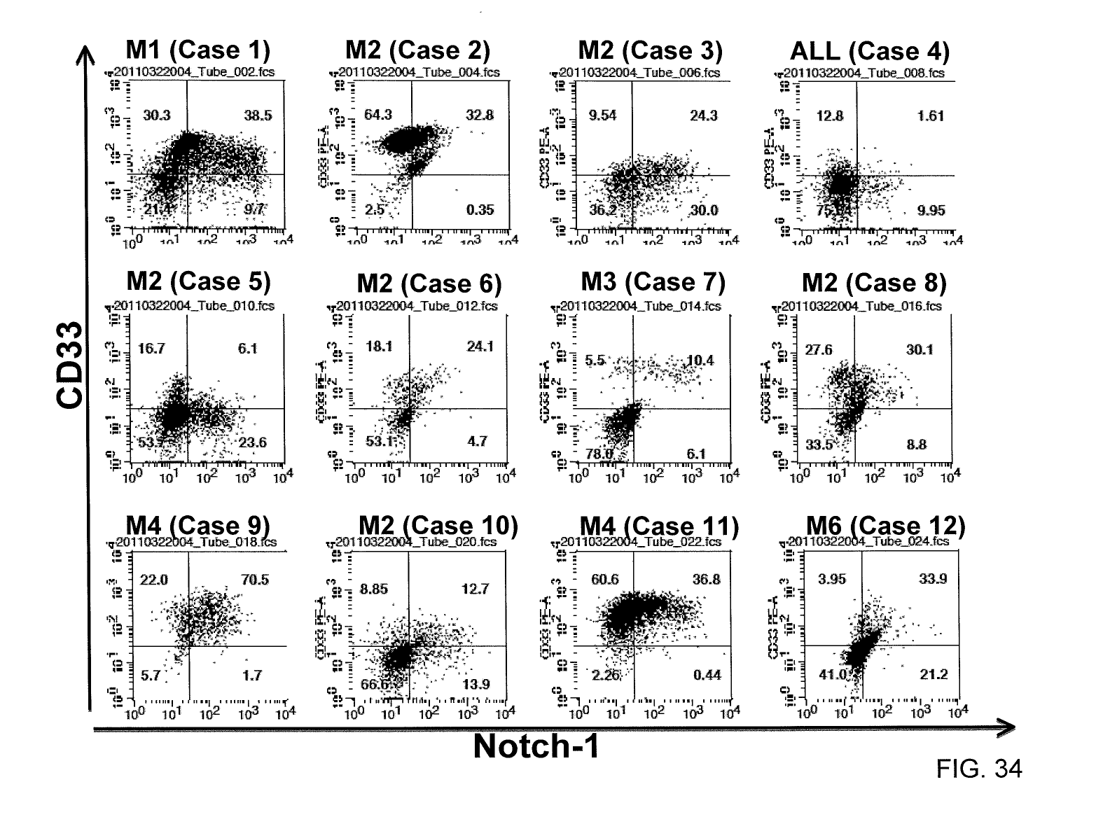

1×10⁶ cells of each sample were washed with 0.1% BSA/PBS, and labeled by the reaction for 10 min with 10 μl of antibodies (PE-conjugated CD33 antibody (R&D) and FITC-conjugated Notch-1 antibody (R&D)) in 1 ml of PBS, then the cells were washed with PBS and the cell suspension was prepared. This cell suspension was subjected to FACSCalibur™ flow cytometer (BD Biosiences, San Jose, Calif., USA) to detect positive cells, and they were analyzed by CellQuest Pro software (BD Biosciences). As demonstrated by the results shown in FIG. 34, Notch-1/CD33-positive cells were frequently observed in the acute myeloid leukemia (AML) samples except one sample of acute lymphatic leukemia (ALL). For reference, the ratios of Notch-1 and/or CD33-positive cells in each sample were listed in Table 9 below. In the table, values of 20 or more are enclosed by rectangles.

TABLE 9

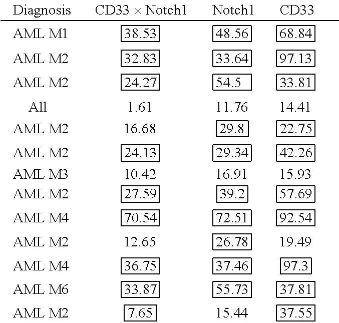

### Example 26

### Effects of Doxorubicin-Encapsulated Fucosylated Liposomes on Various Leukemia Sample Cells

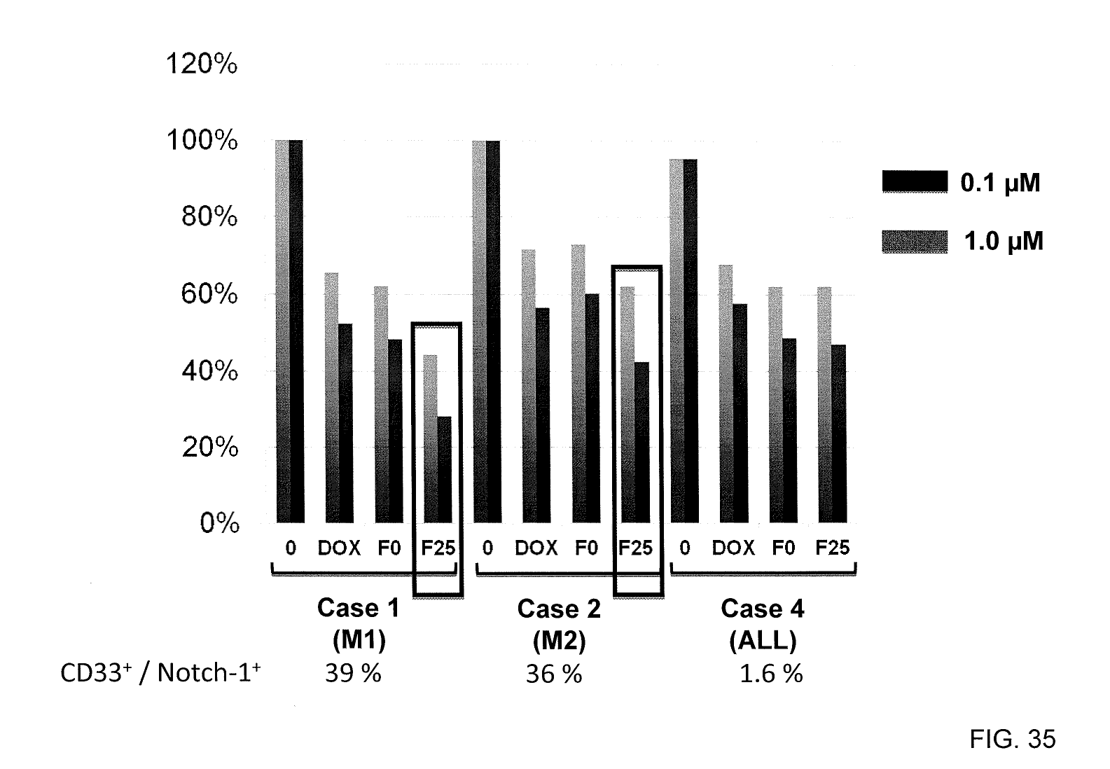

Using WST-1 assay, cytotoxic effects of doxorubicin-encapsulated fucosylated liposomes were investigated. 2×10⁴ cells of each type were seeded in a 96-well culture flask, and the cells were incubated with 0.1 μM or 1.0 μM of doxorubicin alone (DOX), doxorubicin-included fucosylated liposomes (F25) or doxorubicin-included non-fucosylated liposomes (F0). After 2 hr incubation, the cells were washed with PBS, and after replacement of the culture solution (10% FBS-supplemented RPMI1640 medium), further 72 hr of culturing was performed; then an assay using WST-1 reagent was performed similarly to Example 12, and viable cells were counted. As shown in FIG. 35, in the Notch-1 expressing samples (Cases and 2), doxorubicin-included fucosylated liposomes exhibited a significant cytocidal effect compared to doxorubicin alone or doxorubicin-included non-fucosylated liposomes. In contrast, in the Notch-1 non-expressing sample (Case 4), such a significant cytocidal effect was not observed.

1. A carrier targeting fucosylated molecule-producing cells, which comprises an effective amount of fucose for targeting said cells.

2. The carrier according to claim 1, wherein the fucose is L-fucose.

3. The carrier according to claim 1, wherein the fucosylated molecule comprises a type I sugar chain.

4. The carrier according to claim 1, wherein the fucosylated molecule comprises O-linked fucose.

5. The carrier according to claim 1, wherein the fucosylated molecule-producing cell expresses a fucosyltransferase.

6. The carrier according to claim 5, wherein the fucosyltransferase is selected from the group consisting of FUT1, FUT2, FUT3, FUT4, FUT5, FUT6, FUT7, FUT8, FUT9, FUT10, FUT11, POFUT1, and POFUT2.

7. The carrier according to claim 1, wherein the carrier has a form selected from polymer micelle, liposome, emulsion, microsphere, and nanosphere.

8. The carrier according to claim 7, wherein the carrier has a form of liposome, and the molar ratio of the fucose to the lipid contained in the liposome is 8:1-1:8.

9. A composition comprising the carrier according to claim and a drug that controls the activity or growth of fucosylated molecule-producing cells.

10. The composition according to claim 9, wherein the drug that controls the activity or growth of fucosylated molecule-producing cells is selected from the group consisting of anti-inflammatory agents and antitumor agents.

11. The composition according to claim 9, wherein the composition is prepared by mixing the drug and the carrier at a site of clinical practice or its vicinity.

12. A composition comprising the carrier according to claim 1 and a label.

13. The composition according to claim 12, wherein the label is selected from the group consisting of a gas or a substance that generates a gas under physiological conditions, a radioisotope, a magnetic substance, a nuclear magnetic resonance element, a substance that affects the relaxation time of a nuclear magnetic resonance element, a substance that binds to a labeling substance, a fluorescent substance, a fluorophore, a chemiluminescent substance, an enzyme, biotin or its derivative, avidin or its derivative, or a substance comprising one or more thereof.

14. A preparation kit for the composition according to claim 9, containing one or more containers that contain a drug that controls the activity or growth of fucosylated molecule-producing cells, a source of fucose, and as necessary, a carrier-constitutive substance other than fucose, singly or in a combination thereof.

15. A method for treating a disease related to fucosylated molecule-producing cells, comprising administering to a subject in need thereof the composition according to claim 9 in an amount effective for treating said disease.

16. The method according to claim 15, wherein the disease is selected from the group consisting of neoplastic diseases and inflammatory diseases.

17. The method according to claim 16, wherein the neoplastic disease is selected from the group consisting of solid tumors and leukemia.

18. A method for detecting fucosylated molecule-producing cells in a subject, comprising administering to the subject in need thereof the composition according to claim 12 in an amount effective for the detection.

19. The method according to claim 18, wherein the cell is detected by imaging.

20. The method according to claim 18, wherein the cell is selected from the group consisting of neoplastic cells and inflammatory cells.

21. A method for diagnosing a disease related to fucosylated molecule-producing cells, comprising administering to a subject in need thereof the composition according to claim 12 in an amount effective for detection.

22. A method for delivering a substance to fucosylated molecule-producing cells, utilizing the carrier according to claim 1.
